(19) United States
(12) Patent Application Publication
(10) Pub. No.: US 2025/0095860 A1
(43) Pub. Date: Mar. 20, 2025

(54) COMPUTING SYSTEM FOR MEDICAL DATA PROCESSING

(71) Applicant: Bionic Health, Inc., Durham, NC (US)

(72) Inventors: Robert C. Allen, Durham, NC (US); Jared W. Pelo, Durham, NC (US)

(21) Appl. No.: 18/888,437

(22) Filed: Sep. 18, 2024

Related U.S. Application Data
(60) Provisional application No. 63/539,149, filed on Sep. 19, 2023.

(51) Int. Cl. G16H 50/30; G16H 10/60; G16H 20/10; G16H 20/30; G16H 20/60; G16H 20/70

(52) U.S. Cl. CPC G16H 50/30; G16H 10/60; G16H 20/10; G16H 20/30; G16H 20/60; G16H 20/70

(57) ABSTRACT

Disclosed within are methods and systems for method for personalized medical data processing. One method includes receiving, by a health assistant computing entity, member-specific health data from a plurality of member computing entities via a network and processing the received health data using machine learning and artificial intelligence algorithms executed by the health assistant computing entity to generate personalized health insights. The processed health data and generated insights are stored in a private database associated with the automated health assistant computing entity and the processed data and insights are used to generate a dynamic wellness plan for each member. Communication between the member computing entities and professional computing entities are facilitated to implement the wellness plan and the member's health data is continuously monitored and the wellness plan is dynamically updated based on new data inputs. Real-time feedback and recommendations are provided to the member through the member computing entities.

### CROSS-REFERENCE TO RELATED APPLICATIONS

This application claims the benefit of U.S. Provisional Patent Application No. 63/539,149 entitled “COMPUTING SYSTEM FOR MEDICAL DATA PROCESSING” filed on Sep. 19, 2023, the entire content of which is incorporated by reference herein.

### BACKGROUND

### Technical Field of the Invention

The disclosed subject matter relates to data processing and more particularly to a computing system for data processing.

### Description of Related Art

Current approaches to providing healthcare are not designed to optimize health and wellness. For example, traditional healthcare often uses typical ranges for various biomarkers rather than personalized values specific to each individual patient. No two bodies are exactly alike, yet traditional healthcare may group patients together. Traditional healthcare is also designed for addressing symptoms rather than root causes and only mending what is broken rather than holistically improving someone's health. This results in fragmented care where specialists do not coordinate with each other for the same patient. Not only is this inefficient, but it is also ineffective at looking at the whole picture, connecting the dots for the patient, and helping them focus on the right things.

Accordingly, a need exists for improved methods and systems for processing medical data to optimize health and wellness through a personalized action plan.

### SUMMARY

Disclosed within are methods and systems for method for personalized medical data processing. One method includes receiving, by a health assistant computing entity, member-specific health data from a plurality of member computing entities via a network and processing the received health data using machine learning and artificial intelligence algorithms executed by the health assistant computing entity to generate personalized health insights. The processed health data and generated insights are stored in a private database associated with the automated health assistant computing entity and the processed data and insights are used to generate a dynamic wellness plan for each member. Communication between the member computing entities and professional computing entities are facilitated to implement the wellness plan and the member's health data is continuously monitored and the wellness plan is dynamically updated based on new data inputs. Real-time feedback and recommendations are provided to the member through the member computing entities. The features and advantages described in this summary and the following detailed description are not all-inclusive. Many additional features and advantages will be apparent to one of ordinary skill in the art in view of the drawings, specification, and claims presented herein

### BRIEF DESCRIPTION OF THE SEVERAL VIEWS OF THE DRAWING(S)

FIG. 1 is a schematic block diagram of an embodiment of a computing system;

FIG. 2 is a schematic block diagram of an embodiment of an AI health assistant;

FIG. 3A is a schematic block diagram of an embodiment of a computing entity according to an embodiment of the subject matter described herein;

FIG. 3B is a schematic block diagram of an embodiment of a computing entity according to an embodiment of the subject matter described herein;

FIG. 3C is a schematic block diagram of an embodiment of a computing entity according to an embodiment of the subject matter described herein;

FIG. 3D is a schematic block diagram of an embodiment of a computing entity according to an embodiment of the subject matter described herein;

FIG. 3E is a schematic block diagram of an embodiment of a computing entity according to an embodiment of the subject matter described herein;

FIG. 4A is a schematic block diagram of an embodiment of a of computing device that forms at least a portion of a computing entity according to an embodiment of the subject matter described herein;

FIG. 4B is a schematic block diagram of an embodiment of a of computing device that forms at least a portion of a computing entity according to an embodiment of the subject matter described herein;

FIG. 4C is a schematic block diagram of an embodiment of a of computing device that forms at least a portion of a computing entity according to an embodiment of the subject matter described herein;

FIG. 4D is a schematic block diagram of an embodiment of a of computing device that forms at least a portion of a computing entity according to an embodiment of the subject matter described herein;

FIG. 4E is a schematic block diagram of an embodiment of a of computing device that forms at least a portion of a computing entity according to an embodiment of the subject matter described herein;

FIG. 4F is a schematic block diagram of an embodiment of a of computing device that forms at least a portion of a computing entity according to an embodiment of the subject matter described herein;

FIG. 4G is a schematic block diagram of an embodiment of a of computing device that forms at least a portion of a computing entity according to an embodiment of the subject matter described herein;

FIG. 5 is a schematic block diagram of an embodiment of a database;

FIG. 6 is a schematic block diagram of another embodiment of computing system having an AI health assistant computing entity;

FIG. 7 is a schematic block diagram of an example of operation of computing system having an AI health assistant computing entity;

FIG. 8 is a schematic block diagram of another example of operation of computing system having an AI health assistant computing entity;

FIG. 9A is a schematic block diagram of another example of operation of computing system having an AI health assistant computing entity;

FIG. 9B is a schematic block diagram of another example of operation of computing system having an AI health assistant computing entity;

FIG. 10 is a schematic block diagram of an example of communication tracking within a computing system having an AI health assistant computing entity;

FIG. 11 is a schematic block diagram of another example of communication tracking within a computing system having an AI health assistant computing entity;

FIG. 12A is a schematic block diagram of another example of operation of computing system having an AI health assistant computing entity;

FIG. 12B is a schematic block diagram of another example of operation of computing system having an AI health assistant computing entity;

FIG. 13 is a schematic block diagram of another example of operation of computing system having an AI health assistant computing entity;

FIG. 14 is a schematic block diagram of another example of operation of computing system having an AI health assistant computing entity;

FIG. 15A is a schematic block diagram of another example of operation of computing system having an AI health assistant computing entity;

FIG. 15B is a schematic block diagram of another example of a communication prompt suggestion within a computing system having an AI health assistant computing entity;

FIG. 15C is a logic diagram of another example of a communication guardrail within a computing system having an AI health assistant computing entity;

FIG. 15D is a schematic block diagram of another example of operation of computing system having an AI health assistant computing entity;

FIG. 16 is a schematic block diagram of an example of a team of supporting computing entities for a member of computing system having an AI health assistant computing entity;

FIG. 17 is a schematic block diagram of another example of a team of supporting computing entities for a member of computing system having an AI health assistant computing entity;

FIG. 18 is a schematic block diagram of another example of a team of supporting computing entities for a member of computing system having an AI health assistant computing entity;

FIG. 19 is a schematic block diagram of another example of a team of supporting computing entities for a member of computing system having an AI health assistant computing entity;

FIG. 20A is a schematic block diagram of another example of data communication and processing within a computing system having an AI health assistant computing entity;

FIG. 20B is a schematic block diagram of another example of data communication and processing within a computing system having an AI health assistant computing entity;

FIG. 21A is a schematic block diagram of an example of wellness plan graph as communicated and/or processed within a computing system having an AI health assistant computing entity;

FIG. 21B is a schematic block diagram of another example of wellness plan graph as communicated and/or processed within a computing system having an AI health assistant computing entity;

FIG. 21C is a schematic block diagram of another example of wellness plan graph as communicated and/or processed within a computing system having an AI health assistant computing entity;

FIG. 21D is a schematic block diagram of another example of wellness plan graph as communicated and/or processed within a computing system having an AI health assistant computing entity;

FIG. 21E is a schematic block diagram of another example of wellness plan graph as communicated and/or processed within a computing system having an AI health assistant computing entity; and

FIG. 21F is a schematic block diagram of another example of wellness plan graph as communicated and/or processed within a computing system having an AI health assistant computing entity.

FIG. 22 is a schematic block diagram of an example of processing and/or communication of a wellness plan within a computing system having an AI health assistant computing entity;

FIG. 23 is a schematic block diagram of another example of processing and/or communication of a wellness plan within a computing system having an AI health assistant computing entity;

FIG. 24 is a schematic block diagram of another example of processing and/or communication of a day of a wellness plan within a computing system having an AI health assistant computing entity;

FIG. 25 is a schematic block diagram of another example of processing and/or communication information of a wellness plan within a computing system having an AI health assistant computing entity;

FIG. 26 is a schematic block diagram of another example of processing and/or communication information of a wellness plan within a computing system having an AI health assistant computing entity;

FIG. 27 is a schematic block diagram of another example of processing and/or communication information of a wellness plan within a computing system having an AI health assistant computing entity;

FIG. 28 is a schematic block diagram of an embodiment of computing system having an AI health assistant computing entity that processes behavioral health monitoring data;

FIG. 29 is a schematic block diagram of another embodiment of computing system having an AI health assistant computing entity that processes behavioral health monitoring data;

FIG. 30 is a schematic block diagram of an embodiment of computing system having an AI health assistant computing entity that processes lab test recommendation data;

FIG. 31 is a schematic block diagram of another embodiment of computing system having an AI health assistant computing entity that processes lab test recommendation data;

FIG. 32 is a schematic block diagram of an embodiment of computing system having an AI health assistant computing entity that processes asynchronous telemedicine triage data;

FIG. 33 is a schematic block diagram of another embodiment of computing system having an AI health assistant computing entity that processes asynchronous telemedicine triage data;

FIG. 34 is a schematic block diagram of an embodiment of computing system having an AI health assistant computing entity that processes health risk assessment data;

FIG. 35 is a schematic block diagram of another embodiment of computing system having an AI health assistant computing entity that processes health risk assessment data;

FIG. 36 is a schematic block diagram of an embodiment of computing system having an AI health assistant computing entity that processes query of a professional data;

FIG. 37 is a schematic block diagram of another embodiment of computing system having an AI health assistant computing entity that processes query of a professional data;

FIG. 38 is a schematic block diagram of an embodiment of computing system having an AI health assistant computing entity that processes time sequence embedding data;

FIG. 39 is a schematic block diagram of another embodiment of computing system having an AI health assistant computing entity that processes time sequence embedding data;

FIG. 40 is a schematic block diagram of an embodiment of computing system having an AI health assistant computing entity that processes updating of knowledge base data;

FIG. 41 is a schematic block diagram of another embodiment of computing system having an AI health assistant computing entity that processes updating of knowledge base data;

FIG. 42 is a schematic block diagram of an embodiment of computing system having an AI health assistant computing entity that processes linking of messages with background data;

FIG. 43 is a schematic block diagram of another embodiment of computing system having an AI health assistant computing entity that processes linking of messages with background data;

FIG. 44 is a schematic block diagram of an embodiment of computing system having an AI health assistant computing entity that processes vector-based record matching data;

FIG. 45 is a schematic block diagram of another embodiment of computing system having an AI health assistant computing entity that processes vector-based record matching data;

FIG. 46 is a schematic block diagram of an embodiment of computing system having an AI health assistant computing entity that processes interoperability of data;

FIG. 47 is a schematic block diagram of another embodiment of computing system having an AI health assistant computing entity that processes interoperability of data;

FIG. 48 is a schematic block diagram of an embodiment of computing system having an AI health assistant computing entity that processes customization of knowledge base data;

FIG. 49 is a schematic block diagram of another embodiment of computing system having an AI health assistant computing entity that processes customization of knowledge base data;

FIG. 50 is a schematic block diagram of an embodiment of computing system having an AI health assistant computing entity that processes scanning to expand knowledge base data;

FIG. 51 is a schematic block diagram of another embodiment of computing system having an AI health assistant computing entity that processes scanning to expand knowledge base data;

FIG. 52 is a schematic block diagram of an example of a medical data storage structure within a computing system having an AI health assistant computing entity;

FIG. 53 is a schematic block diagram of an example of a vascular disease data storage structure within a computing system having an AI health assistant computing entity;

FIG. 54 is a schematic block diagram of an example of a physical fitness data storage structure within a computing system having an AI health assistant computing entity;

FIG. 55 is a schematic block diagram of an example of a nutrition data storage structure within a computing system having an AI health assistant computing entity;

FIG. 56 is a schematic block diagram of an example of displaying a member's data within a computing system having an AI health assistant computing entity; and

FIG. 57 is a schematic block diagram of another example of displaying a member's data within a computing system having an AI health assistant computing entity.

### DETAILED DESCRIPTION OF THE INVENTION

Using AI, specifically a large language model (LLM), to facilitate and augment communication between a member patient and a group of medical professionals may involve several steps. Below is an overview of a process for facilitating and augmenting these communications according to one embodiment of the subject matter described herein.

The process may begin with the patient initiating communication, which could be through a messaging platform, email, or another digital interface such as a dedicated mobile app or a webapp. The LLM collects and processes the text of these messages, extracting key details such as symptoms, concerns, medical history, and any specific questions or requests. Database(s) containing additional medical data or health information may also be queried to supplement the details extracted from the messages.

Once messages are received, the LLM may categorize them into relevant categories. Common categories might include symptoms and health concerns, medication and treatment questions, appointment scheduling, and general inquiries. The LLM uses its understanding of medical terminology and context to ensure accurate categorization, often leveraging pre-trained models specific to healthcare or custom-trained models tailored to medical communication nuances.

After categorizing the messages, the LLM predicts the most appropriate next message based on the context and historical communication patterns between the patient and medical professionals. For instance, if the message describes symptoms, the LLM might draft questions to gather more information or suggest possible next steps. For medication inquiries, it might provide information on dosages or side effects, or recommend contacting a pharmacist. For appointment scheduling, it might propose available time slots or guide the patient through the scheduling process. General inquiries might be directed to relevant resources or answered with standard information.

The LLM drafts responses or suggestions for the medical professionals to review. These drafts are based on the predicted needs and context of the patient's message. Medical professionals then review these drafts, making necessary adjustments to ensure accuracy, clarity, and appropriateness before sending them to the patient.

Following the communication, feedback from both the patient and medical professionals is collected. This feedback, which may include how helpful the responses were or any issues encountered, is used to refine the LLM's categorization and response prediction algorithms. Over time, this iterative process improves the LLM's accuracy and better aligns it with the specific needs and preferences of the medical practice and its patients.

As communication continues, the LLM adapts to new information and evolving patient needs, learning from ongoing interactions to handle future communications more effectively. For a more integrated approach, the LLM can also be connected to electronic health records (EHR) to provide even more personalized and contextually relevant responses based on the patient's medical history.

In this way, the systems and methods disclosed herein can significantly streamline and enhance communication between a patient and medical professionals by categorizing messages, predicting next steps, and generating or augmenting responses. However, in some embodiments, medical professionals may review and finalize communications to ensure accuracy and adherence to medical standards.

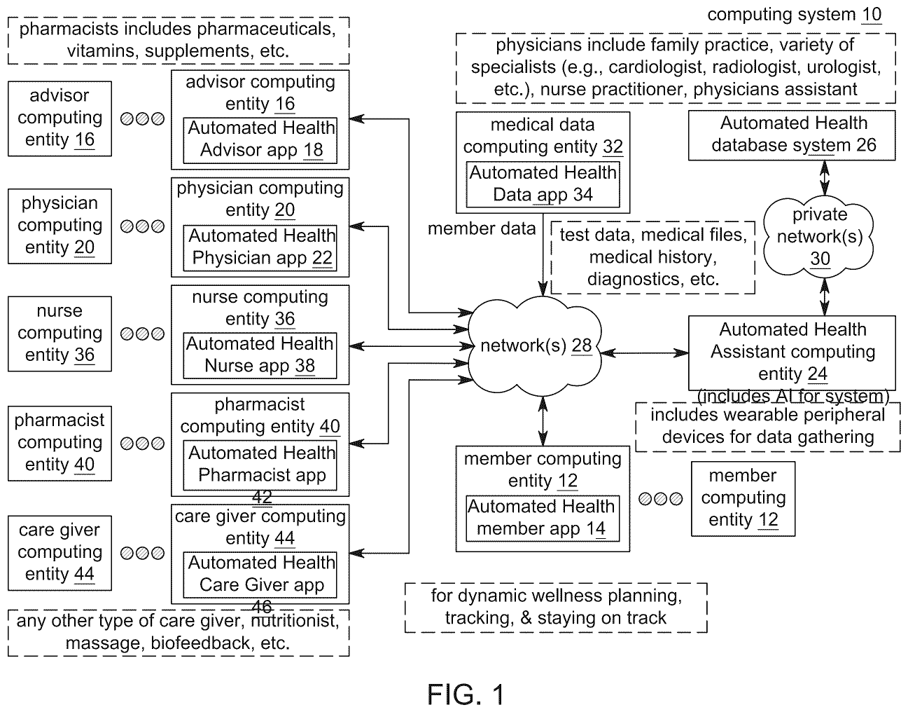

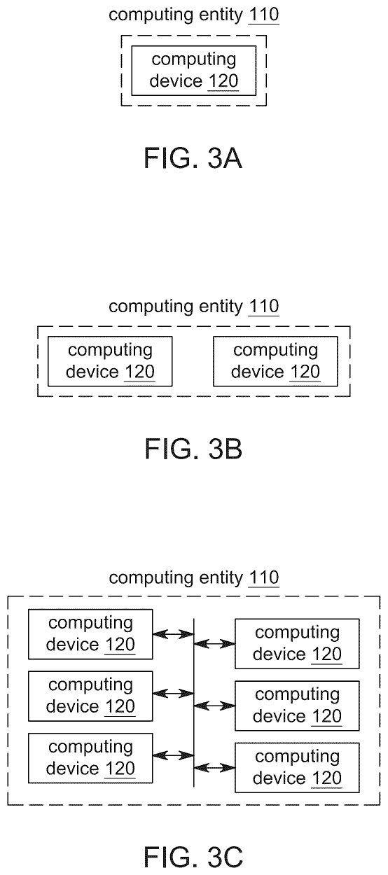

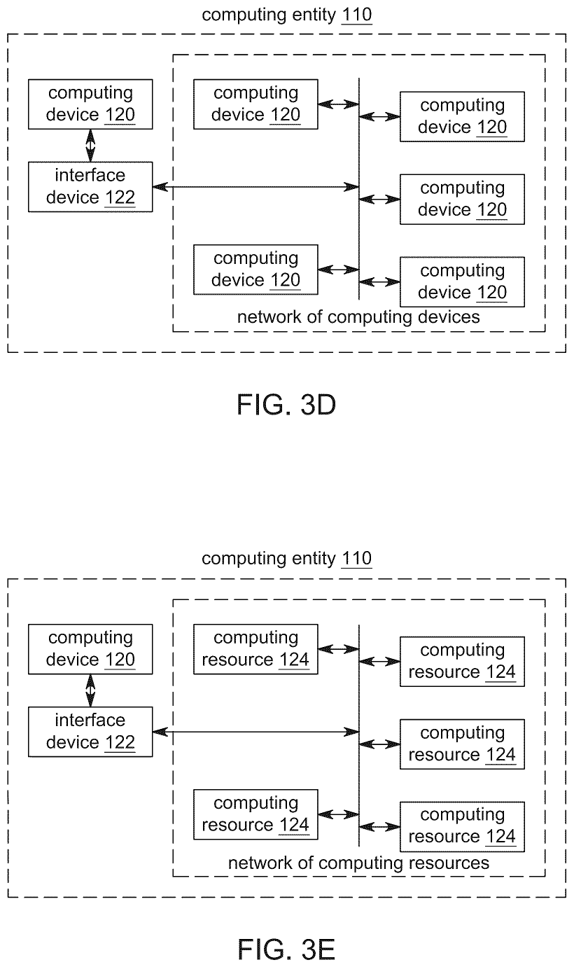

FIG. 1 is a schematic block diagram of an embodiment of a computing system 10 that includes a plurality of member computing entities 12, a plurality of advisor computing entities 16, a plurality of physician computing entities 20, an automated (e.g., fusion of biology and technology) health assistant computing entity 24, an automated health database system 26, one or more medical data computing entities 32, a plurality of nurse computing entities 36, a plurality of pharmacist computing entities 40, and a plurality of care giver computing entities 44. A computing entity is implemented in accordance with an embodiment of FIGS. 3A-3E.

The member computing entities 12 communicate with the assistant computing entity 24 via one or more networks 28 (e.g., the internet, cellular telephone network, a wide area network, etc.). A member computing entity 12 accesses the network(s) directly or via a local area network (LAN) (e.g., a Wi-Fi). A member computing entity 12 communicates with an advisor computing entity 16, a physician computing entity 20, a medical data computing entity 32, a nurse computing entity 36, a pharmacist computing entity 40, and/or a care giver computing entity 44 via the network(s) 28 and the assistant computing entity 24.

What makes a computing entity a particular type of computing entity is the Automated Health software it executes. For example, a member computing entity 12 stores and executes an automated member application 14, which includes a plurality of software programs that facilitates a member's dynamic wellness plan, tracking it, executing it, and communication with a team of medical professionals.

As another example, an advisor computing entity 16 stores and executes an automated advisor application 18, which includes a plurality of software programs that facilitates a qualified person's ability to be an advisor, such as a life coach. An advisor is a highly trained health coach, specializing in supporting members through habit change and helping members shape a life that feels fulfilling and vibrant to them. They play a pivotal role in ensuring that a health plan aligns with a member's personal goals, particularly as they relate to, for example, a member's 4th quarter aspirations. They are partners in identifying what a well-lived life looks like to each member, and together, forge the path to get there.

As another example, a physician computing entity 20 stores and executes an automated physician application 22, which includes a plurality of software programs that facilitates a physician's interaction with the assistant computing entity 24 and/or with members via their member computing entities. A physician may be a specific type of physician and the automated physician application 22 may be adapted accordingly. For example, a family practice physician would have an automated physician application 22 geared towards family practice medicine. Other specific physicians include, but are not limited to, cardiologist, radiologist, urologist, proctologist, orthopedist, chiropractor, acupuncturist, nurse practitioner, and physician's assistant.

As another example, a medical data computing entity 32 stores and executes an automated medical data application 34, which includes a plurality of software programs that facilitates gathering of data and/or communication of data with the assistant computing entity 24 and/or with a member via a member computing entity. In this example, the medical data computing entity 32 is affiliated a medical data facility such as a doctor's office (for a member's medical records, a radiologist report, etc.), a hospital, a blood analysis lab, an imaging center (e.g., x-ray, MRI, CT scan, etc.), a test clinic, etc.

As another example, a nurse computing entity 36 stores and executes an automated nurse application 38, which includes a plurality of software programs that facilitates a nurse's interaction with the assistant computing entity 24 and/or with members via their member computing entities. As another example, a pharmacist computing entity 40 stores and executes an automated pharmacist application 42, which includes a plurality of software programs that facilitates a pharmacist's interaction with the assistant computing entity 24 and/or with members via their member computing entities. Under the heading of pharmacist includes pharmaceuticals, supplements, vitamins, and other ingestible and/or topical treatment modalities.

As another example, a care giver computing entity 44 stores and executes an automated care giver application 46, which includes a plurality of software programs that facilitates a care giver's interaction with the assistant computing entity 24 and/or with members via their member computing entities. There are a variety of care givers and the automated care giver application 46 would be adapted accordingly. For example, a physical therapist would have an automated care giver application geared towards physical therapy. Other care givers include, but are not limited to, nutritionist, massage therapist, biofeedback, etc.

The automated health assistant computing entity 24 is a focal point of the machine learning and artificial intelligence of the computing system. The assistant computing entity 24 couples to the automated health database system 26 via a private network 30. The private network may be implemented in a variety of ways. For example, the private network 30 is a direction connection between data communication interfaces of the databases of the database system 26 and data communication interfaces of the assistant computing entity 24. As another example, the private network is a virtual private network (VPN) operating on the network 28. As yet another example, the private network is a private LAN, a private WLAN, or other private network configuration.

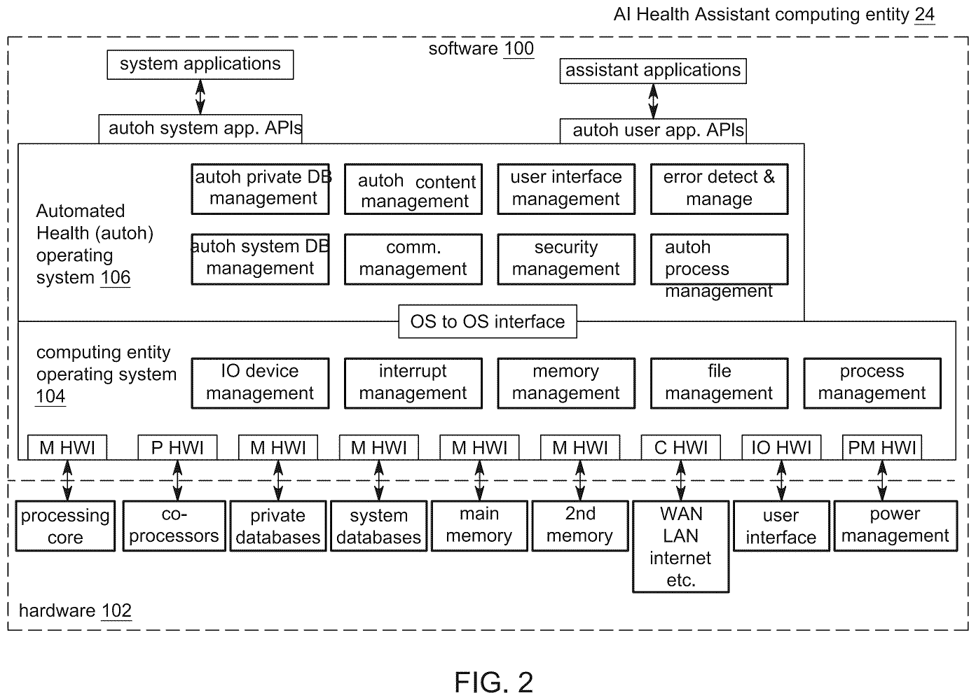

FIG. 2 is a schematic block diagram of an embodiment of an AI health assistant computing entity 24. The computing entity 24 includes an improved computer architecture to support the machine learning and/or artificial intelligence programs it executes to facilitate member wellness functions of the computing system 10. As shown, the computing entity 24 includes a software section 100 and a hardware section 102. Note that the software programs of the software section are stored in memory of the hardware section 102.

The software section 100 includes a computing entity operation system 104, an automated health operating system 106, a plurality of system applications, and a plurality of assistant applications. The computing entity operating system includes IO device management, interrupt management, memory management, file management, and process management. The automated health (autoh) operating system includes private database (DB) management (of a member, of a physician), autoh content management, autoh user interface management (of members, physicians, nurses, advisors, etc.), error detect & management, system database management, communication management (between AI assistant and members, between AI assistant and professionals, etc.), security management, and process management (of autoh data processing).

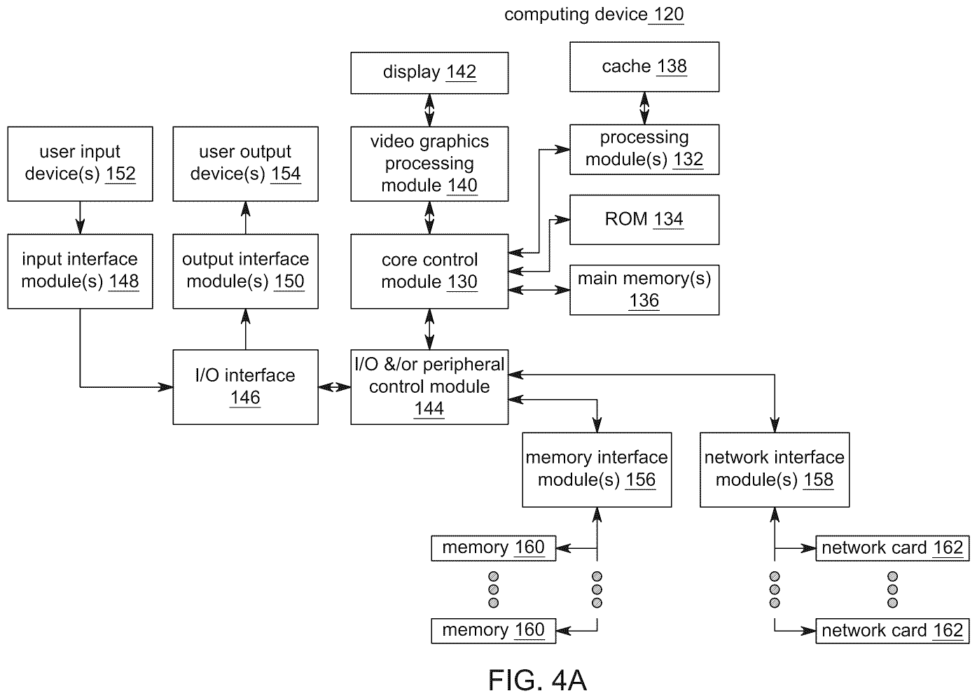

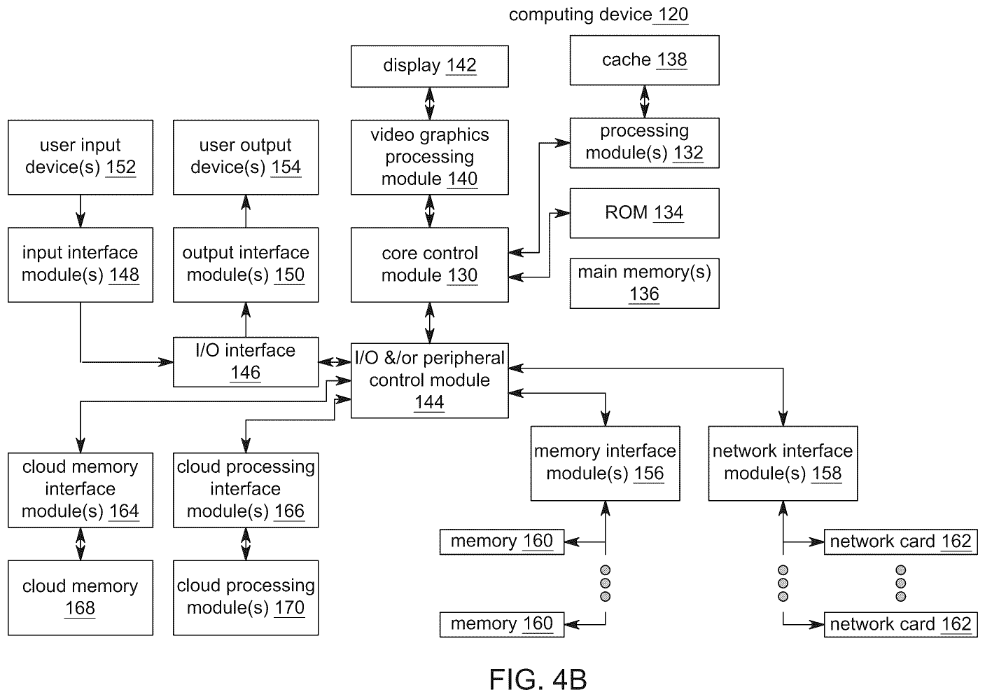

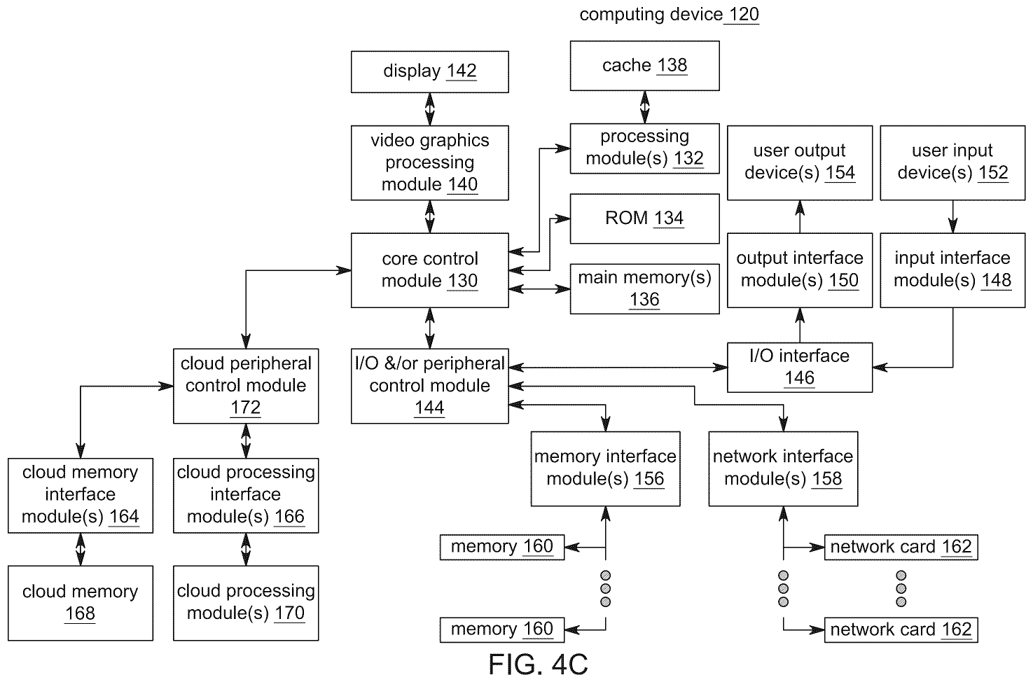

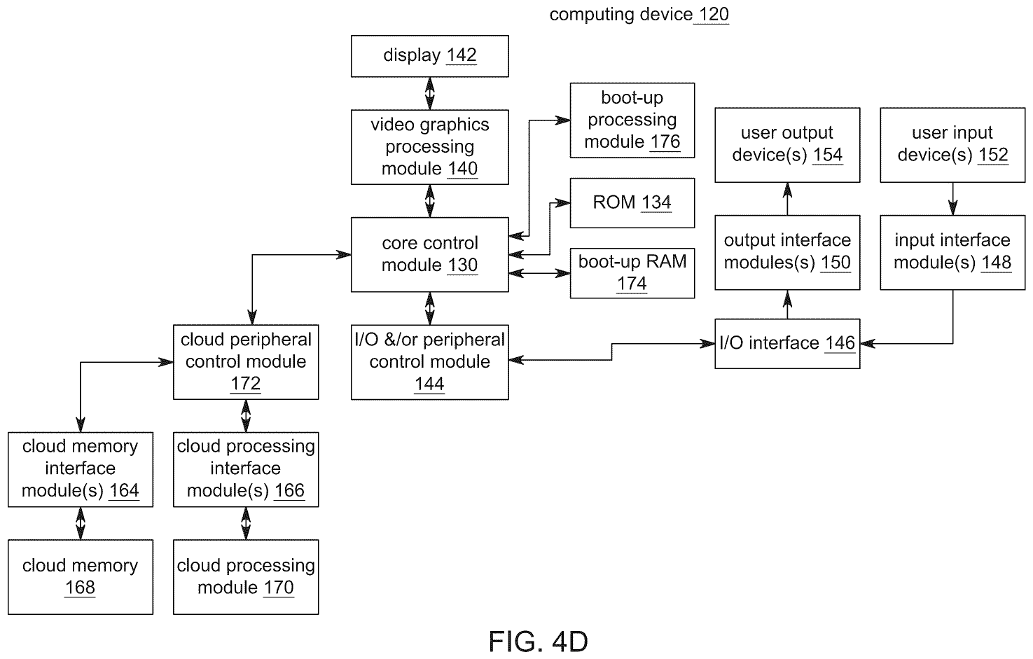

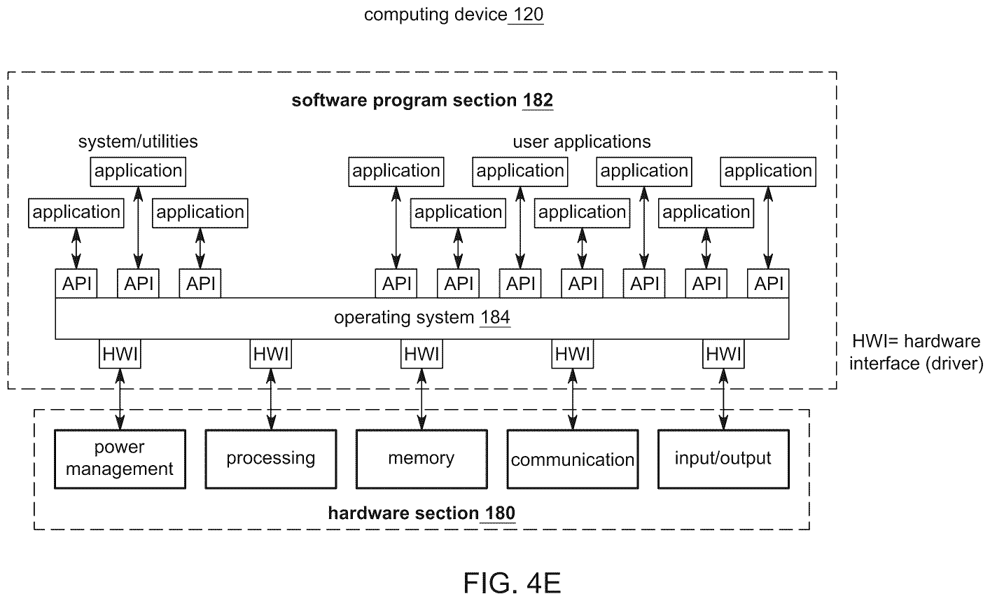

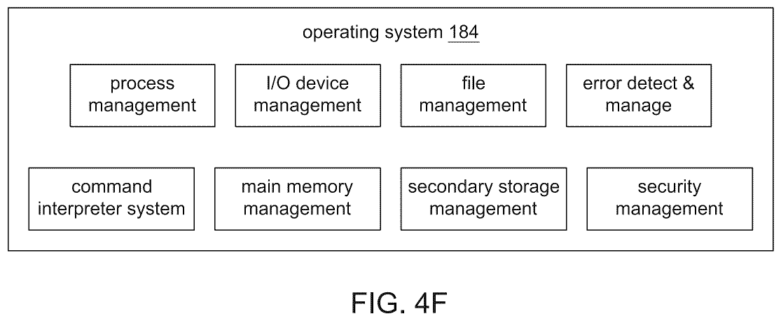

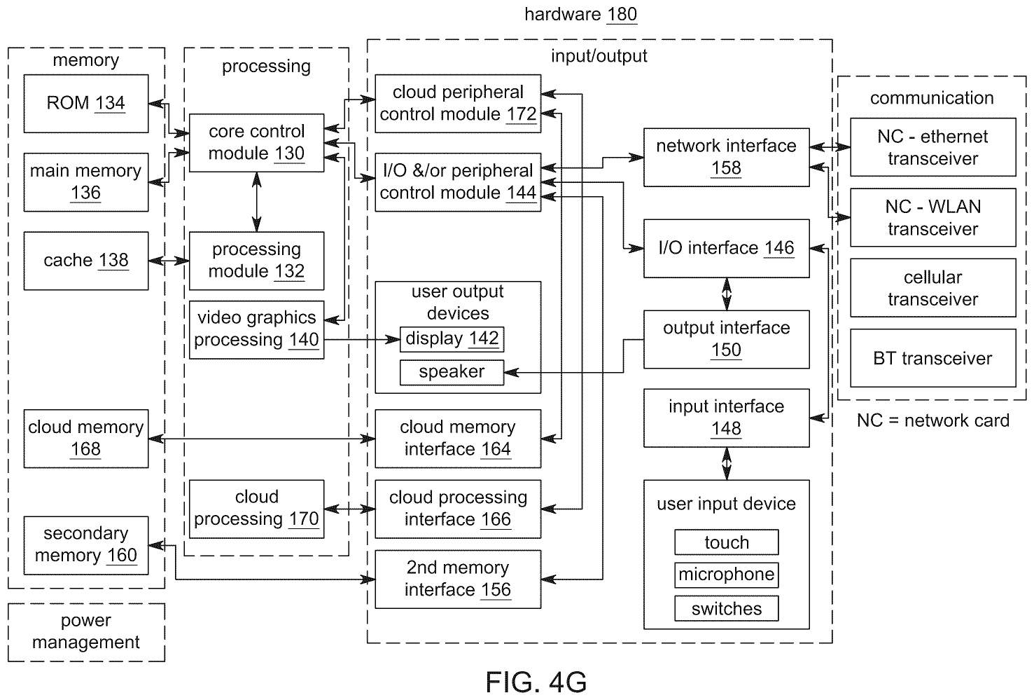

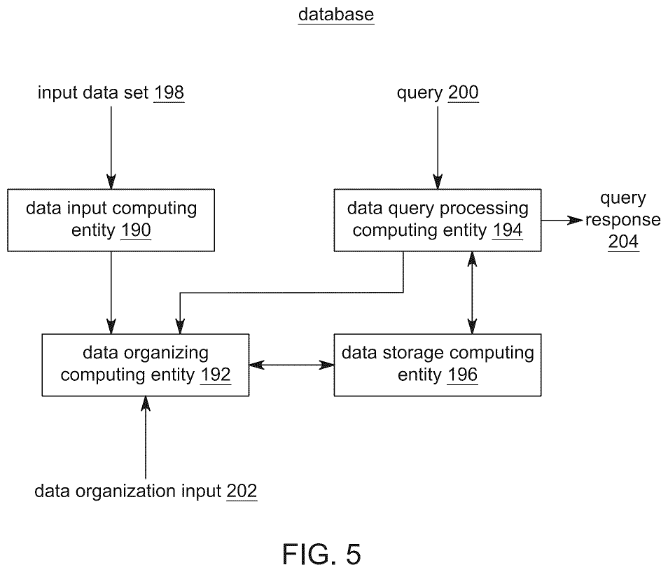

The hardware section 102 includes a processing core, one or more co-processors, a private database, one or more system databases, main memory, secondary memory, one or more network connections (WAN, LAN, internet, etc.), user interface, and power management. The hardware section 102 is implemented as shown in one or more of FIGS. 4A-4G and the databases as shown in FIG. 5.

The AI assistant computing entity includes an improved computer architecture, provides many new technologies that provides many significant technical improvements and/or technical advantages over existing computers and computer programs that support the conventional patent process. These include, but are not limited to, Education, which involves providing essential information to individuals, enabling them to understand their bodies and the potential health risks they might encounter and is an important initial step in promoting active participation in managing one's health. These also include, Data Analysis, which involves gathering relevant health data and interpreting it in a meaningful way to make the complex nature of the human body understandable through a clear presentation of data. Insights is a component that involves using technology to derive useful insights from the analyzed data. These insights serve as a guide for making informed decisions and strategies regarding health management. Treatment is an implementation stage where the knowledge, data, and insights previously collected are used to develop personalized treatment plans. These plans are designed to minimize health risks, improve overall health, and maintain vitality. These plans also function as a bridge between high-tech and nature's wisdom. Use of AI and technology humanizes healthcare by creating a health system where humans and technology work synergistically. A state-of-the-art digital platform is designed to put members in control of their health journey. A member application is a hub for personal health data, it connects a member to a personal Automated Health Advisor and Automated Health Physician, and it is an interface with the Automated Health Assistant-AI that powers automated health. The system incorporates information from multiple disciplines, learning from each data point to become more accurate and personalized to your health needs. The more relevant data it has, the smarter it becomes, fine-tuning its recommendations to each member. Such perpetual learning further melds nature's wisdom with technological advancement. The system does not just react to illness but works actively to keep members healthy. The system is continually learning, improving, and using existing and pieces of relevant data to provide better care regarding illness recovery, illness prevention, and overall wellness. The system predicts potential health problems for a member and prevent them before they occur. The system includes a holistic digital platform designed to help members navigate their health journey with precision and confidence. The system includes tools designed to keep members on track, making the execution of their health plan not just a possibility but a reality. The system proactively, via personalized health management, functions to improve a member's current life and the last 25 years of life, so they are spent in health and vitality rather than illness and decline. The system distills raw, high-quality data into actionable & in-depth insights that pave the way for personalized treatments. The system verifies the quality of the data it collects to ensure accuracy of its insights and the effectiveness of treatments derived therefrom. The system prioritizes data resources to carefully curate the valuable and insightful data for each module. These range from lab tests and biometrics to wearable tech data and personal health surveys.

Like other computers, the AI assistant computing entity with an improved computer architecture operates at the machine level where data is represented as unique sequences of l's and O's. For the AI assistant computing entity with an improved computer architecture data is one of data operands, operational instructions, or resulting data, where a set of operational instructions (or operational codes, or op codes) is performed on one or more data operands to produce resulting data. Within the AI assistant computing entity with an improved computer architecture it is common for the resulting data of a previously executed set of operational instructions to be a data operand(s) [intermediate operand] for a subsequently executed set of operational instructions, where a set includes one or more operational instructions.

Operational instructions are programming language specific and provide the instructions for the computer to read data operands, write data operands, write data results, and/or perform a function on a data operand(s). Examples of functions includes, but are not limited to, add, subtract, shift left, shift right, multiply, divide, a logic AND, a logic OR, a logic XOR, etc. A generic format for an operational instruction is [function; address for operand; (additional address for additional operands if functions involve two or more operands); address of where to write the data result].

The data operands, operational instructions, and data results are stored within the improved computer's memory as unique sequences of l's and O's. How the l's and O's are stored, retrieved, and processed within the computer dictate whether they are related to a data operand, an operational instruction, or a data result. All of the unique sequences of l's and O's regarding a program need to be properly associated with data operands, operational instructions, and data result; and need to be retrieved, processed, and/or stored in a precise manner for the program to operating correctly (e.g., produce the desired data result(s) from one or more initial data operands and/or one or more intermediate data operands).

A new combination and/or ordering of operational instructions that is executed by the improved computer on data operands (new, known, initial, and/or intermediate) to produce a new data result is novel. The creation, storage, and/or execution of operational instructions by an improved computer on data operands to produce a new data result are, in it of themselves, technical challenges.

Creating a new tool via the novel programming of a computer provides the benefits of any physical tool, which include, but are not limited to, improving performance, efficiency, accurately, reliability, safety, resolution, etc. of an existing task, providing new solutions to an existing task, and providing solutions to new tasks.

The human meaning of the l's and O's of the data operands, the operational instructions, and/or the data results does not change the technical challenges of programming a computer to produce an output, or outputs, through one or more sets of operational instructions operating on initial data operands and/or intermediate operands.

FIGS. 3A through 3E are schematic block diagram of embodiments of computing entities that form at least part of an improved computer for technology. FIG. 3A is schematic block diagram of an embodiment of a computing entity 110 that includes a computing device 120 (e.g., one or more of the embodiments of FIGS. 4A-4G). A computing device may function as a user computing device, a server, a system computing device, a data storage device, a data security device, a networking device, a user access device, a cell phone, a tablet, a laptop, a printer, a game console, a satellite control box, a cable box, etc.

FIG. 3B is schematic block diagram of an embodiment of a computing entity 110 that includes two or more computing devices 120 (e.g., two or more from any combination of the embodiments of FIGS. 4A-4G). The computing devices 120 perform the functions of a computing entity in a peer processing manner (e.g., coordinate together to perform the functions), in a master-slave manner (e.g., one computing device coordinates and the other support it), and/or in another manner.

FIG. 3C is schematic block diagram of an embodiment of a computing entity 110 that includes a network of computing devices 120 (e.g., two or more from any combination of the embodiments of FIGS. 4A-4G). The computing devices are coupled together via one or more network connections (e.g., WAN, LAN, cellular data, WLAN, etc.) and perform the functions of the computing entity.

FIG. 3D is schematic block diagram of an embodiment of a computing entity 110 that includes a primary computing device (e.g., any one of the computing devices of FIGS. 4A-4G), an interface device (e.g., a network connection), and a network of computing devices 120 (e.g., one or more from any combination of the embodiments of FIGS. 4A-4G). The primary computing device utilizes the other computing devices as co-processors to execute one or more the functions of the computing entity, as storage for data, for other data processing functions, and/or storage purposes.

FIG. 3E is schematic block diagram of an embodiment of a computing entity 110 that includes a primary computing device (e.g., any one of the computing devices of FIGS. 4A-4G), an interface device (e.g., a network connection) 122, and a network of computing resources 124 (e.g., two or more resources from any combination of the embodiments of FIGS. 4A-4G). The primary computing device utilizes the computing resources as co-processors to execute one or more the functions of the computing entity, as storage for data, for other data processing functions, and/or storage purposes.

FIGS. 4A through 4G are schematic block diagram of embodiments of computing devices that form at least a portion of a computing entity. FIG. 4A is a schematic block diagram of an embodiment of a computing device 120 that includes a plurality of computing resources. The computing resources, which form a computing core, include one or more core control modules 130, one or more processing modules 132, one or more main memories 136, a read only memory (ROM) 134 for a boot up sequence, cache memory 138, one or more video graphics processing modules 140, one or more displays 142 (optional), an Input-Output (I/O) peripheral control module 144, an I/O interface module 146 (which could be omitted if direct connect IO is implemented), one or more input interface modules 148, one or more output interface modules 150, one or more network interface modules 158, and one or more memory interface modules 156.

A processing module 132 is described in greater detail at the end of the detailed description section and, in an alternative embodiment, has a direction connection to the main memory 136. In an alternate embodiment, the core control module 130 and the I/O and/or peripheral control module 144 are one module, such as a chipset, a quick path interconnect (QPI), and/or an ultra-path interconnect (UPI).

The processing module 132, the core module 130, and/or the video graphics processing module 140 form a processing core for a computer computing. Additional combinations of processing modules 132, core modules 130, and/or video graphics processing modules 140 form co-processors for the improved computer for technology. Computing resources 124 of FIG. 4E include one more of the components shown in this Figure and/or in or more of FIGS. 4B through 4G.

Each of the main memories 136 includes one or more Random Access Memory (RAM) integrated circuits, or chips. In general, the main memory 136 stores data and operational instructions most relevant for the processing module 132. For example, the core control module 130 coordinates the transfer of data and/or operational instructions between the main memory 136 and the secondary memory device(s) 160. The data and/or operational instructions retrieve from secondary memory 160 are the data and/or operational instructions requested by the processing module or will most likely be needed by the processing module. When the processing module is done with the data and/or operational instructions in main memory, the core control module 130 coordinates sending updated data to the secondary memory 160 for storage.

The secondary memory 160 includes one or more hard drives, one or more solid state memory chips, and/or one or more other large capacity storage devices that, in comparison to cache memory and main memory devices, is/are relatively inexpensive with respect to cost per amount of data stored. The secondary memory 160 is coupled to the core control module 130 via the I/O and/or peripheral control module 144 and via one or more memory interface modules 156. In an embodiment, the I/O and/or peripheral control module 144 includes one or more Peripheral Component Interface (PCI) buses to which peripheral components connect to the core control module 130. A memory interface module 156 includes a software driver and a hardware connector for coupling a memory device to the I/O and/or peripheral control module 144. For example, a memory interface 156 is in accordance with a Serial Advanced Technology Attachment (SATA) port.

The core control module 130 coordinates data communications between the processing module(s) 132 and network(s) via the I/O and/or peripheral control module 144, the network interface module(s) 158, and one or more network cards 162. A network card 160 includes a wireless communication unit or a wired communication unit. For example, a wireless communication unit includes a wireless local area network (WLAN) communication device, a cellular communication device, a Bluetooth device, and/or a ZigBee communication device. For example, a wired communication unit includes a Gigabit LAN connection, a Firewire connection, and/or a proprietary computer wired connection. A network interface module 158 includes a software driver and a hardware connector for coupling the network card to the I/O and/or peripheral control module 144. For example, the network interface module 158 is in accordance with one or more versions of IEEE 802.11, cellular telephone protocols, 10/100/1000 Gigabit LAN protocols, etc.

The core control module 130 coordinates data communications between the processing module(s) 132 and input device(s) 152 via the input interface module(s) 148, the I/O interface 146, and the I/O and/or peripheral control module 144. An input device 152 includes a keypad, a keyboard, control switches, a touchpad, a microphone, a camera, etc. An input interface module 148 includes a software driver and a hardware connector for coupling an input device to the I/O and/or peripheral control module 144. In an embodiment, an input interface module 148 is in accordance with one or more Universal Serial Bus (USB) protocols.

The core control module 130 coordinates data communications between the processing module(s) 132 and output device(s) 154 via the output interface module(s) 150 and the I/O and/or peripheral control module 144. An output device 154 includes a speaker, auxiliary memory, headphones, etc. An output interface module 150 includes a software driver and a hardware connector for coupling an output device to the I/O and/or peripheral control module 144. In an embodiment, an output interface module 150 is in accordance with one or more audio codec protocols.

The processing module 132 communicates directly with a video graphics processing module 140 to display data on the display 142. The display 142 includes an LED (light emitting diode) display, an LCD (liquid crystal display), and/or other type of display technology. The display has a resolution, an aspect ratio, and other features that affect the quality of the display. The video graphics processing module 140 receives data from the processing module 132, processes the data to produce rendered data in accordance with the characteristics of the display, and provides the rendered data to the display 142.

FIG. 4B is a schematic block diagram of an embodiment of a computing device 120 that includes a plurality of computing resources similar to the computing resources of FIG. 4A with the addition of one or more cloud memory interface modules 164, one or more cloud processing interface modules 166, cloud memory 168, and one or more cloud processing modules 170. The cloud memory 168 includes one or more tiers of memory (e.g., ROM, volatile (RAM, main, etc.), non-volatile (hard drive, solid-state, etc.) and/or backup (hard drive, tape, etc.)) that is remoted from the core control module and is accessed via a network (WAN and/or LAN). The cloud processing module 170 is similar to processing module 132 but is remote from the core control module and is accessed via a network.

FIG. 4C is a schematic block diagram of an embodiment of a computing device 120 that includes a plurality of computing resources similar to the computing resources of FIG. 4B with a change in how the cloud memory interface module(s) 164 and the cloud processing interface module(s) 166 are coupled to the core control module 130. In this embodiment, the interface modules 164 and 166 are coupled to a cloud peripheral control module 172 that directly couples to the core control module 130.

FIG. 4D is a schematic block diagram of an embodiment of a computing device 120 that includes a plurality of computing resources, which includes include a core control module 130, a boot up processing module 176, boot up RAM 174, a read only memory (ROM) 134, a one or more video graphics processing modules 140, one or more displays 48 (optional), an Input-Output (I/O) peripheral control module 144, one or more input interface modules 148, one or more output interface modules 150, one or more cloud memory interface modules 164, one or more cloud processing interface modules 166, cloud memory 168, and cloud processing module(s) 170.

In this embodiment, the computing device 120 includes enough processing resources (e.g., module 176, ROM 134, and RAM 174) to boot up. Once booted up, the cloud memory 168 and the cloud processing module(s) 170 function as the computing device's memory (e.g., main and hard drive) and processing module.

FIG. 4E is a schematic block diagram of another embodiment of a computing device 120 that includes a hardware section 180 and a software program section 182. The hardware section 180 includes the hardware functions of power management, processing, memory, communications, and input/output. FIG. 4G illustrates the hardware section 180 in greater detail.

The software program section 182 includes an operating system 184, system and/or utilities applications, and user applications. The software program section further includes APIs and HWIs. APIs (application programming interface) are the interfaces between the system and/or utilities applications and the operating system and the interfaces between the user applications and the operating system 184. HWIs (hardware interface) are the interfaces between the hardware components and the operating system. For some hardware components, the HWI is a software driver. The functions of the operating system 184 are discussed in greater detail with reference to FIG. 4F.

FIG. 4F is a diagram of an example of the functions of the operating system of a computing device 120. In general, the operating system function to identify and route input data to the right places within the computer and to identify and route output data to the right places within the computer. Input data is with respect to the processing module and includes data received from the input devices, data retrieved from main memory, data retrieved from secondary memory, and/or data received via a network card. Output data is with respect to the processing module and includes data to be written into main memory, data to be written into secondary memory, data to be displayed via the display and/or an output device, and data to be communicated via a network care.

The operating system 184 includes the OS functions of process management, command interpreter system, I/O device management, main memory management, file management, secondary storage management, error detection & correction management, and security management. The process management OS function manages processes of the software section operating on the hardware section, where a process is a program or portion thereof.

The process management OS function includes a plurality of specific functions to manage the interaction of software and hardware. The specific functions process management OS function may include: loading a process for execution; enabling at least partial execution of a process; suspending execution of a process; resuming execution of a process; terminating execution of a process; loading operational instructions and/or data into main memory for a process; providing communication between two or more active processes; avoiding deadlock of a process and/or interdependent processes; and controlling access to shared hardware components.

The I/O Device Management OS function coordinates translation of input data into programming language data and/or into machine language data used by the hardware components and translation of machine language data and/or programming language data into output data. Typically, input devices and/or output devices have an associated driver that provides at least a portion of the data translation. For example, a microphone captures analog audible signals and converts them into digital audio signals per an audio encoding format. An audio input driver converts, if needed, the digital audio signals into a format that is readily usable by a hardware component.

The File Management OS function coordinates the storage and retrieval of data as files in a file directory system, which is stored in memory of the computing device. In general, the file management OS function may perform the following the specific functions: file creation, editing, deletion, and/or archiving; directory creation, editing, deletion, and/or archiving; memory mapping filed and/or directors to memory locations of secondary memory; and backing up of files and/or directories.

The Network Management OS function manages access to a network by the computing device. Network management may include performing: network fault analysis; network maintenance for quality of service; network access control among multiple clients; and network security upkeep.

The Main Memory Management OS function manages access to the main memory of a computing device. This includes keeping track of memory space usage and which processes are using it; allocating available memory space to requesting processes; and deallocating memory space from terminated processes.

The Secondary Storage Management OS function manages access to the secondary memory of a computing device. This includes free memory space management, storage allocation, disk scheduling, and memory defragmentation.

The Security Management OS function protects the computing device from internal and external issues that could adversely affect the operations of the computing device. With respect to internal issues, the OS function ensures that processes negligibly interfere with each other; ensures that processes are accessing the appropriate hardware components, the appropriate files, etc.; and ensures that processes execute within appropriate memory spaces (e.g., user memory space for user applications, system memory space for system applications, etc.).

The security management OS function also protects the computing device from external issues, such as, but not limited to, hack attempts, phishing attacks, denial of service attacks, bait and switch attacks, cookie theft, a virus, a trojan horse, a worm, click jacking attacks, keylogger attacks, cavesdropping, waterhole attacks, SQL injection attacks, and DNS spoofing attacks.

FIG. 4G is a schematic block diagram of the hardware components of the hardware section 180 of a computing device. The memory portion of the hardware section includes the ROM 134, the main memory 136, the cache memory 138, the cloud memory 168, and the secondary memory 160. The processing portion of the hardware section includes the core control module 130, the processing module 132, the video graphics processing module 140, and the cloud processing module 170.

The input/output portion of the hardware section includes the cloud peripheral control module 172, the I/O and/or peripheral control module 144, the network interface module 158, the I/O interface module 146, the output device interface 150, the input device interface 148, the cloud memory interface module 164, the cloud processing interface module 166, and the secondary memory interface module 156. The IO portion further includes input devices such as a touch screen, a microphone, and switches. The IO portion also includes output devices such as speakers and a display.

The communication portion includes an ethernet transceiver network interface card (NIC), a WLAN network card, a cellular transceiver, a Bluetooth transceiver, and/or any other device for wired and/or wireless network communication.

FIG. 5 is a schematic block diagram of an embodiment of a database that includes a data input computing entity 190, a data organizing computing entity 191, a data query processing computing entity 192, and a data storage computing entity 193. Each of the computing entities is implemented in accordance with one or more of the embodiments of FIGS. 4A through 4E. The data input computing entity 190 is operable to receive an input data set 198. The input data set 198 is a collection of related data that can be represented in a tabular form of columns and rows, and/or other tabular structure. In an example, the columns represent different data elements of data for a particular source and the rows corresponds to the different sources (e.g., employees, licenses, email communications, etc.).

If the data set 198 is in a desired tabular format, the data input computing entity 190 provides the data set to the data organizing computing entity 192. If not, the data input computing entity 190 reformats the data set to put it into the desired tabular format.

The data organizing computing entity 192 organizes the data set 198 in accordance with a data organizing input 202. In an example, the input 202 is regarding a particular query and requests that the data be organized for efficient analysis of the data for the query. In another example, the input 202 instructs the data organizing computing entity 192 to organize the data in a time-based manner. The organized data is provided to the data storage computing entity 196 for storage.

When the data query processing computing entity 194 receives a query 200, it accesses the data storage computing entity 196 regarding a data set for the query. If the data set is stored in a desired format for the query, the data query processing computing entity 192 retrieves the data set and executes the query to produce a query response 204. If the data set is not stored in the desired format, the data query processing computing entity 194 communicates with the data organizing computing entity 192, which re-organizes the data set into the desired format and stores the re-organized data in the data storage computing entity 196.

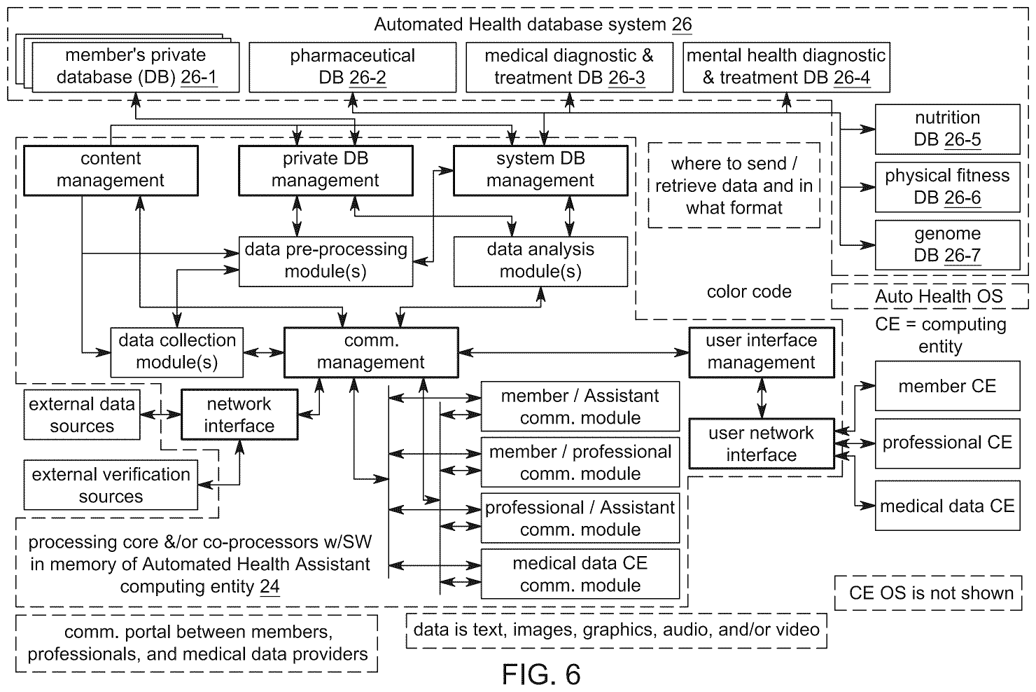

FIG. 6 is a schematic block diagram of another embodiment of computing system having an AI health assistant computing entity. The database system 26 includes a plurality of member's private databases 26-1, a pharmaceutical database 26-2, a medical diagnosis & treatment database 26-3, a mental health diagnostic & treatment database 26-4, a nutrition database 26-5, a physical fitness database 26-6, and a genome database 26-7.

A member's private database 26-1 includes data that is relevant to the member's past health, current health, and future health; the member's wellness objectives; the member's past and current wellness plans as generated by the AI assistant computing entity; plan execution tracking data; health and wellness source materials of interest to the member; and/or specific health and/or wellness topics. Health data includes medical data such as medical diagnosis, medical treatments, blood test results, family medical history, personal medical history, etc.

A pharmaceutical database 26-2 includes data regarding a variety of pharmaceutical products, their uses, their recommended doses, their side effects, their use restrictions, their interactions with other medications, foods, vitamins, and/or supplements. The data may further include costs, who can prescribe them, prescribed usage plan, etc. The pharmaceutical database 26-2, or a supplements database, includes data regarding supplements and/or vitamins, their uses, their recommended doses, their side effects, their use restrictions, their interactions with medications, foods, other vitamins, and/or other supplements.

A medical diagnosis & treatment database 26-3 includes data regarding medical conditions, diagnosis of each of the medical conditions (and/or for a group of like medical conditions), treatment for each of the medical conditions, risk factors for each of the medical conditions (and/or for a group of like medical conditions), prevention of each medical condition (and/or for a group of like medical conditions), etc.

A mental health diagnostic & treatment database 26-4 includes data regarding mental health conditions, diagnosis of each of the mental health conditions (and/or for a group of like mental health conditions), treatment for each of the mental health conditions, risk factors for each of the mental health conditions (and/or for a group of like mental health conditions), prevention of each mental health condition (and/or for a group of like mental health conditions), etc.

A nutrition database 26-5 includes data regarding nutritional information of foods, nutritional information of beverages, nutritional information of supplements, and information regarding a plurality of nutritional protocols.

A physical fitness database 26-6 stores data regarding physical fitness. For example, the data is regarding physical fitness training programs, physical fitness risk factors, supplements to aid physical fitness training, physical fitness products, etc.

A genome database 26-7 stores data and information regarding genomes and genetics. Genetics is regarding the functioning and composition of a single gene. Genomes address the interaction of genes and their influence of the growth and development of a body.

The AI assistant computing entity 24 with an improved computer architecture includes the components as previously discussed. For this discussion, the AI assistant computing entity 24 includes the automated health operating system functions (as highlighted by green boxes) of content management, private DB management, system DB management, communication management, and user interface management. The computing entity 24, which is identified via the red-grey highlighting, further includes the hardware components of a network interface and a user network interface. The computing entity 24 further includes software modules running on the processing core and/or one or more co-processors of data pre-processing, data analysis, data collection, member/AI Assistant communication, member/professional communication, professional/AI Assistant communication, and medical data computing entity/AI Assistant communication.

The AI assistant computing entity 24 is coupled to external data sources and external verification sources via the network interface. The AI assistant is further coupled to one or more member computing entities (CE), one or more professional computing entities, and/or one or more medical data computing entities via the user network interface.

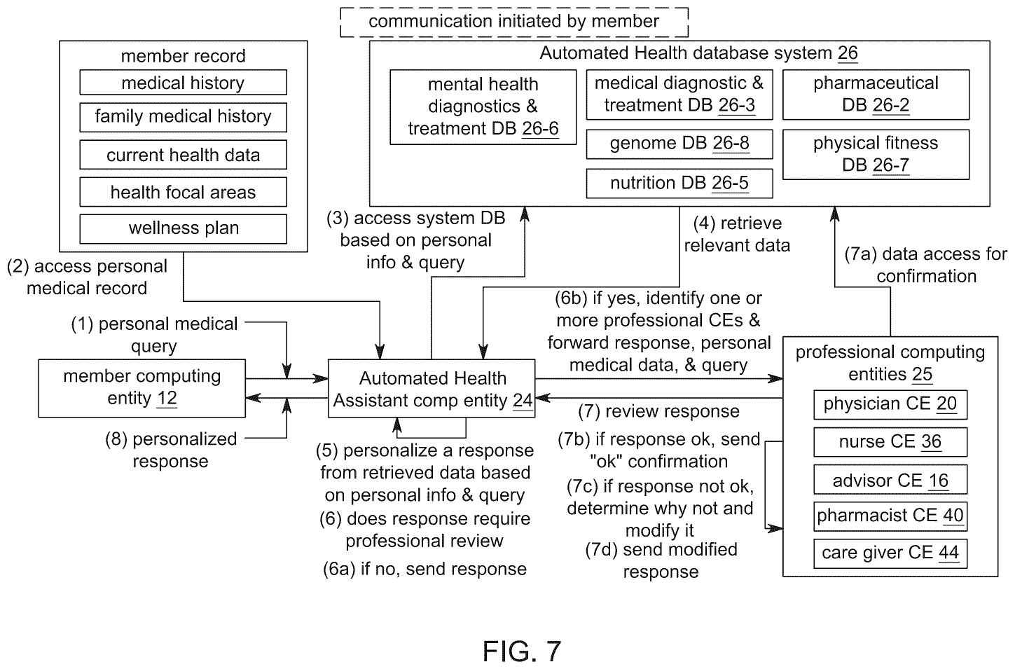

FIG. 7 is a schematic block diagram of an example of operation of computing system having an AI health assistant computing entity. In this example, the AI health assistant computing entity 24 facilitates and AI augments a communication initiated by a member.

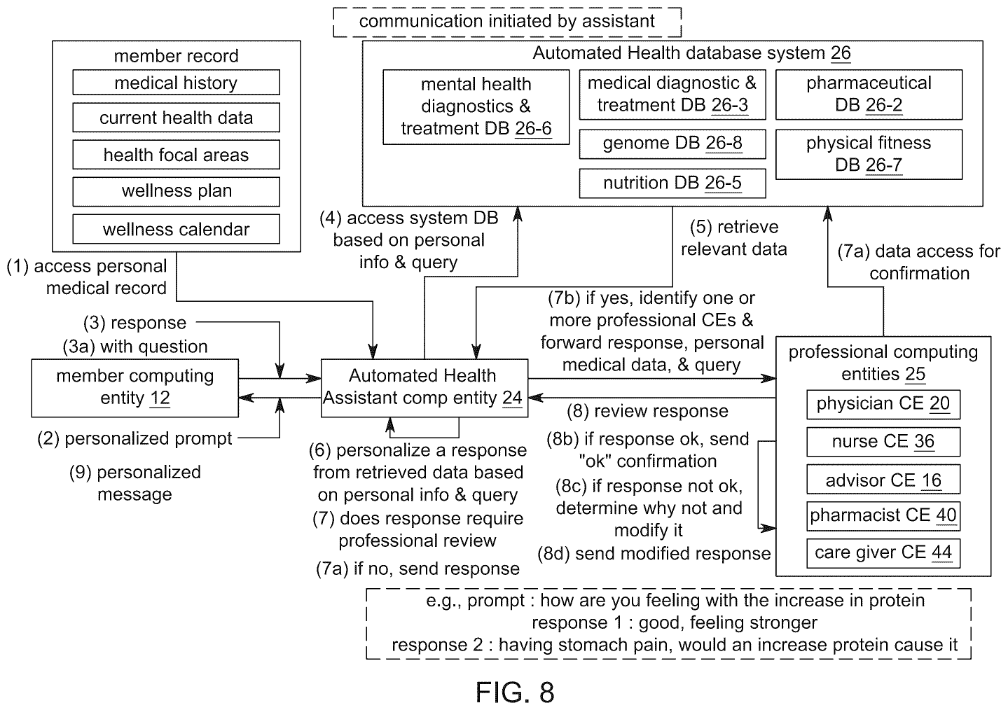

Referring to FIG. 8, the process may begin, at step 1, by the Automated Health Assistant computing entity 24 receiving a personal medical query and, at step 2, accessing the personal medical record of the patient (member record). This may include a medical history, current health data, health focal areas, a wellness plan, and a wellness calendar. Next, the Automated Health Assistant computing entity 24 may generate a personalized prompt utilizing the member's medical record. The Automated Health Assistant computing entity 24 may also access, at step 3, the health database system 26 based on personal information and a query to retrieve relevant data at step 4. The Automated Health Assistant database 26 may include a pharmaceutical database 26-2, a medical diagnostic and treatment database 26-3, a nutrition database 26-5, a mental health diagnosis and treatment database 26-6, and a genome database 26-8.

Once the relevant data is retrieved, Automated Health Assistant computing entity 24 may, at step 5, personalize a response using the retrieved data based on the personal information and query. In some cases, the personalized response may require professional review before sending and in other cases, no additional review is required, and the response may be sent immediately. To determine whether a response requires professional review, the Automated Health Assistant computing entity 24 may identify one or more professionals and forward the response along with any relevant information, such as personal medical data and the query, to Professional Computing entities 25. Professional computing entities 25 may include a physician computing entity 20, a nurse computing entity 36, an advisor computing entity 16, a pharmacist computing entity 40, and a caregiver computing entity 44. The professional computing entities 25 may review the response and, if the response is OK, send an OK confirmation to the Automated Health Assistant computing entity 24. If, however, the response is not OK, then the professional computing entities 25 may determine why not, modify the response, and return the modified response. Finally, the Automated Health Assistant computing entity 24 may provide the personalized message, at step 8, to the member computing entity 12.

FIG. 8 is a schematic block diagram of another example of operation of computing system having an AI health assistant computing entity. In this example, the AI health assistant computing entity 24 facilitates and AI augments a communication with a member regarding a prompt message generated by the AI assistant computing entity.

Referring to FIG. 8, the process may begin by the Automated Health Assistant computing entity 24 accessing the personal medical record of the patient. This may include a medical history, current health data, health focal areas, a wellness plan, and a wellness calendar. Next, the Automated Health Assistant computing entity 24 may generate a personalized prompt utilizing the member's medical record. For example, the generated prompt may be “How are you feeling with the increase in protein?”, which is sent to member computing entity 12. In response, the member computing entity 12 may provide either a response (e.g., “Good, feeling stronger.”) or a response with a question (e.g., “Having stomach pain, would an increase in protein cause that?”).

The Automated Health Assistant computing entity 24 may then access Automated Health Assistant database 26 and query the DB based on personal information of the member. The Automated Health Assistant database 26 may include a pharmaceutical database 26-2, a medical diagnostic and treatment database 26-3, a nutrition database 26-5, a mental health diagnosis and treatment database 26-6, and a genome database 26-8.

Once the relevant data is retrieved, Automated Health Assistant computing entity 24 may personalize a response using the retrieved data based on the personal information and query. In some cases, the personalized response may require professional review before sending and in other cases, no additional review is required, and the response may be sent immediately.

To determine whether a response requires professional review, the Automated Health Assistant computing entity 24 may identify one or more professionals and forward the response along with any relevant information, such as personal medical data and the query, to Professional Computing entities 25. Professional computing entities 25 may include a physician computing entity 20, a nurse computing entity 36, an advisor computing entity 16, a pharmacist computing entity 40, and a caregiver computing entity 44. The professional computing entities 25 may review the response and, if the response is OK, send an OK confirmation to the Automated Health Assistant computing entity 24. If, however, the response is not OK, then the professional computing entities 25 may determine why not, modify the response, and return the modified response. Finally, the Automated Health Assistant computing entity 24 may provide the personalized message to the member computing entity 12.

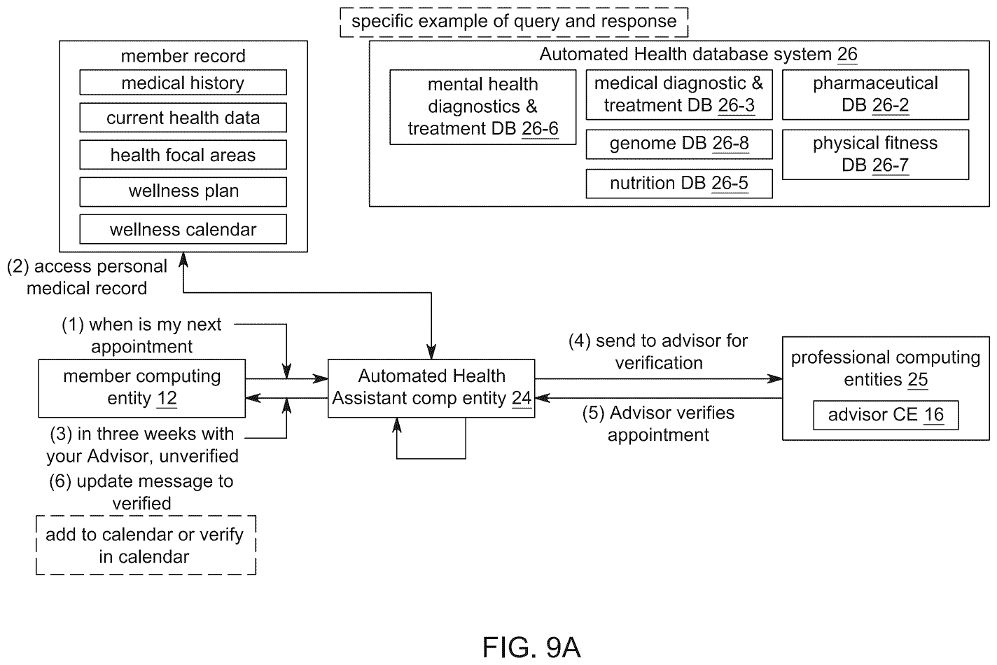

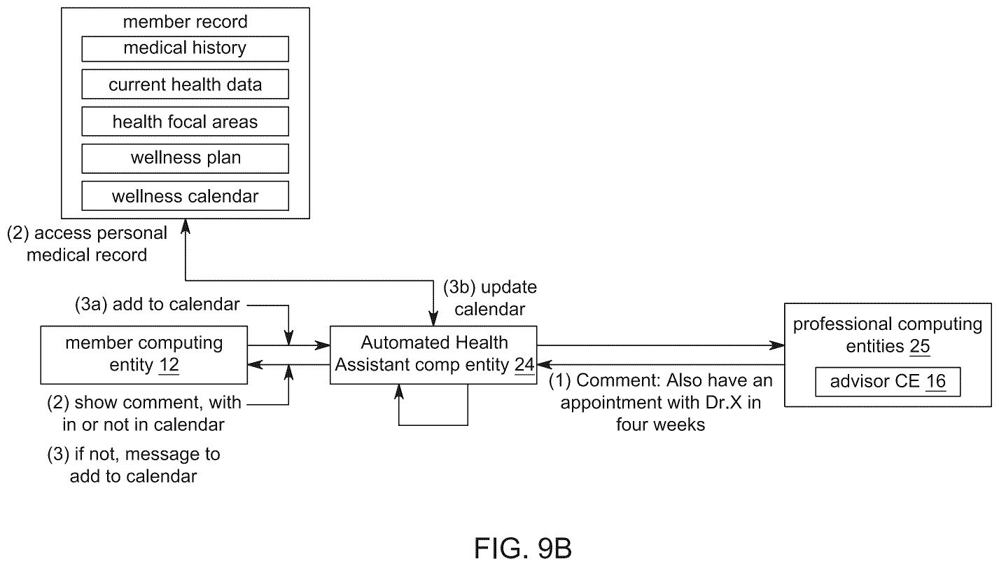

FIG. 9A is a schematic block diagram of another example of operation of computing system having an AI health assistant computing entity. In this example, the AI health assistant computing entity 24 facilitates and AI augments a communication with a member regarding an upcoming appointment query from a member. In this example, the member computing entity 12 may query the Automated Health Assistant computing entity 24 “When is my next appointment?”. The Automated Health Assistant computing entity 24 then accesses the member record for the member and informs the member that their next appointment (unverified) is in three weeks with their Advisor. The Automated Health Assistant computing entity 24 then sends a verification request to the professional computing entities 25 and receives, in response, an Advisor verification of the appointment. In this example, the Advisor verified that the next appointment is in three weeks and, as a result, Automated Health Assistant computing entity 24 updates the message to indicate that it is verified. The Automated Health Assistant computing entity 24 may also add the appointment to the member's calendar.

FIG. 9B is a schematic block diagram of another example of operation of computing system having an AI health assistant computing entity. In this example, the AI health assistant computing entity 24 facilitates and AI augments a communication with a member regarding another upcoming appointment of a member.

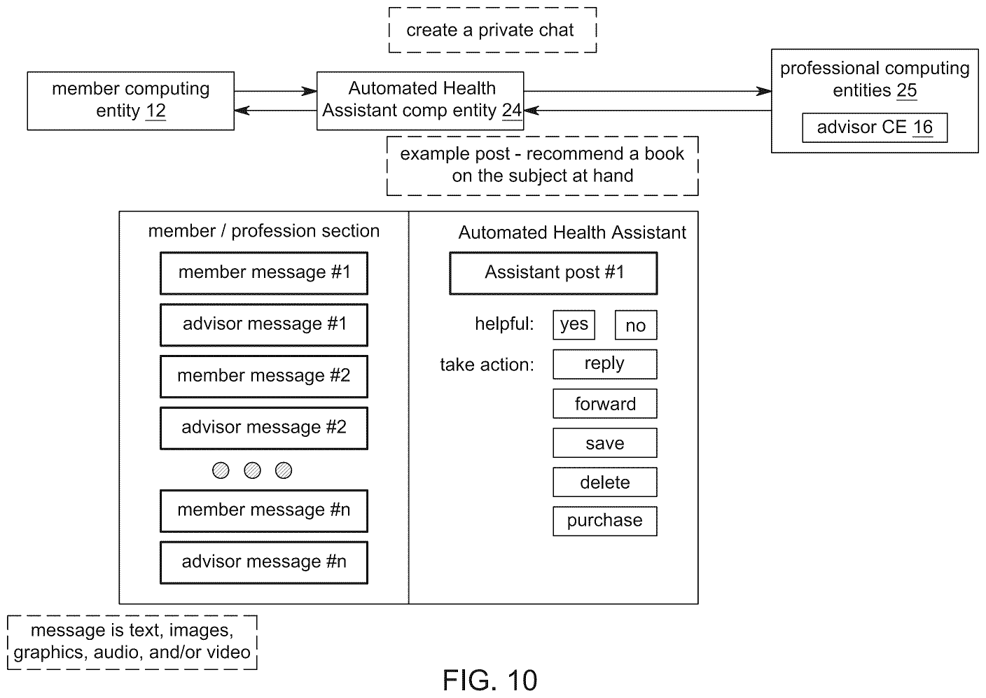

FIG. 10 is a schematic block diagram of an example of communication tracking within a computing system having an AI health assistant computing entity. In this example, the AI health assistant computing entity 24 facilitates, records, and AI augments a communication between a member and an advisor.

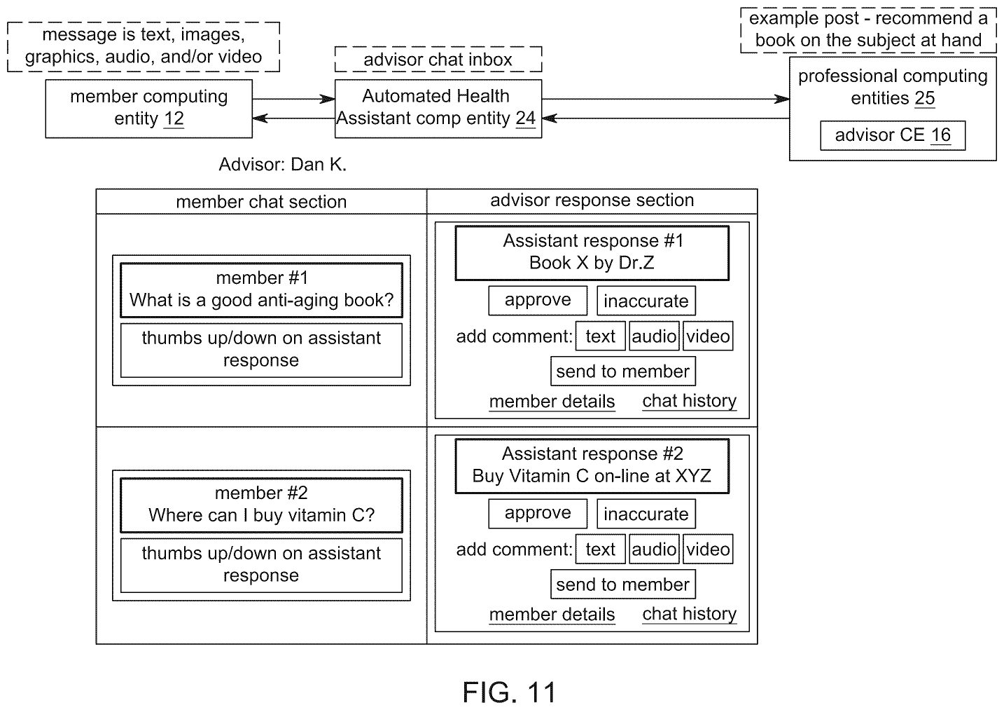

FIG. 11 is a schematic block diagram of another example of communication tracking within a computing system having an AI health assistant computing entity. In this example, the AI health assistant computing entity 24 facilitates, records, tracks, and AI augments communications between a professional and a plurality of members via a chat inbox for the professional.

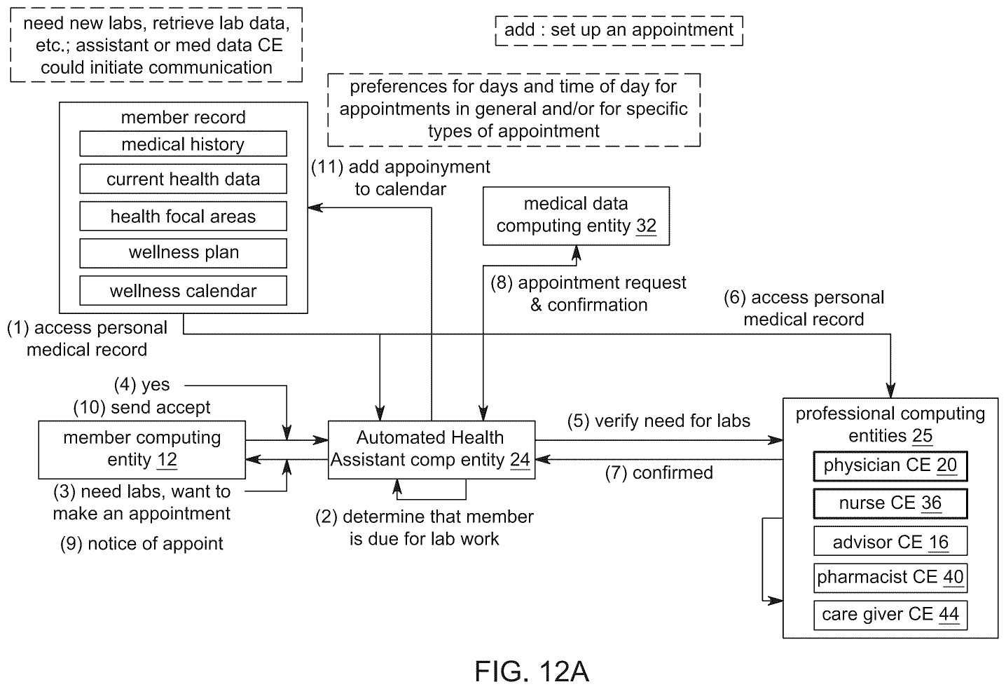

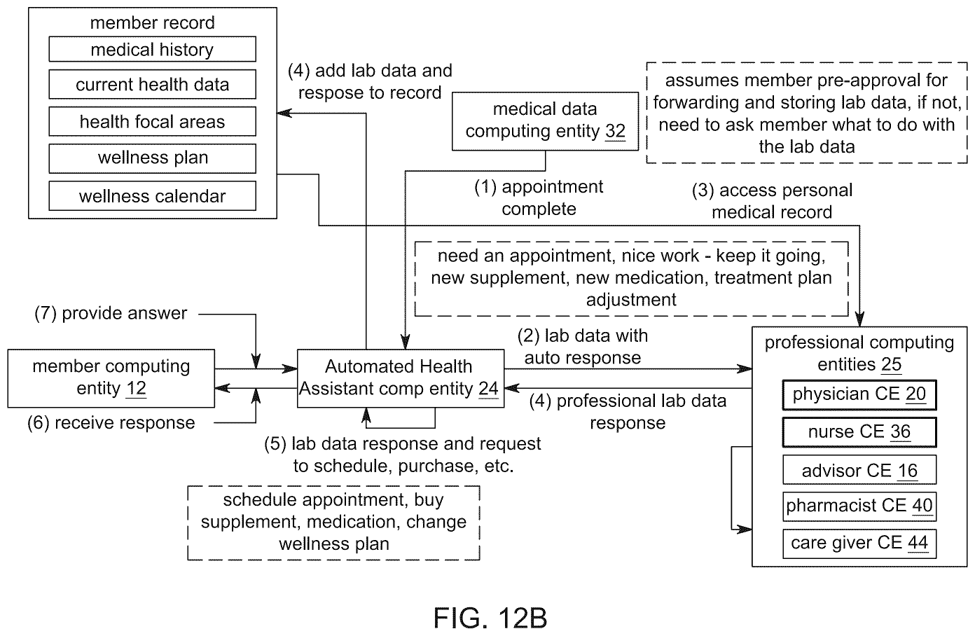

FIG. 12A is a schematic block diagram of another example of operation of computing system having an AI health assistant computing entity. In this example, the AI health assistant computing entity 24 facilitates, records, and AI augments a communication between a member, a professional and a medical data facility regarding a lab data and corresponding appointment for the member.

FIG. 12B is a schematic block diagram of another example of operation of computing system having an AI health assistant computing entity. In this example, the AI health assistant computing entity 24 facilitates, records, and AI augments a communication between a member, a professional and a medical data facility regarding completion of a lab test and the corresponding lab results for the member.

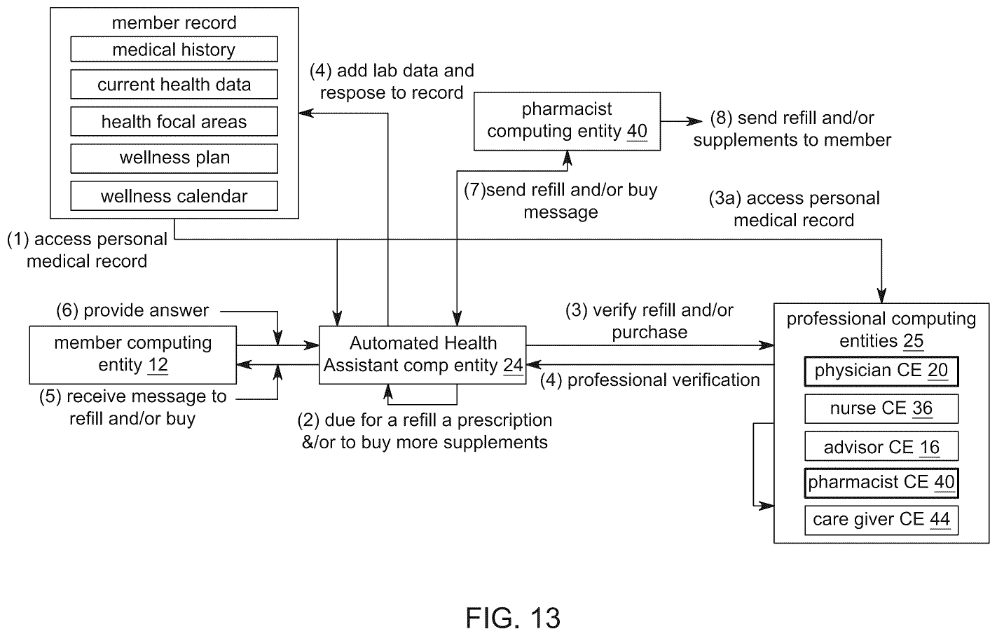

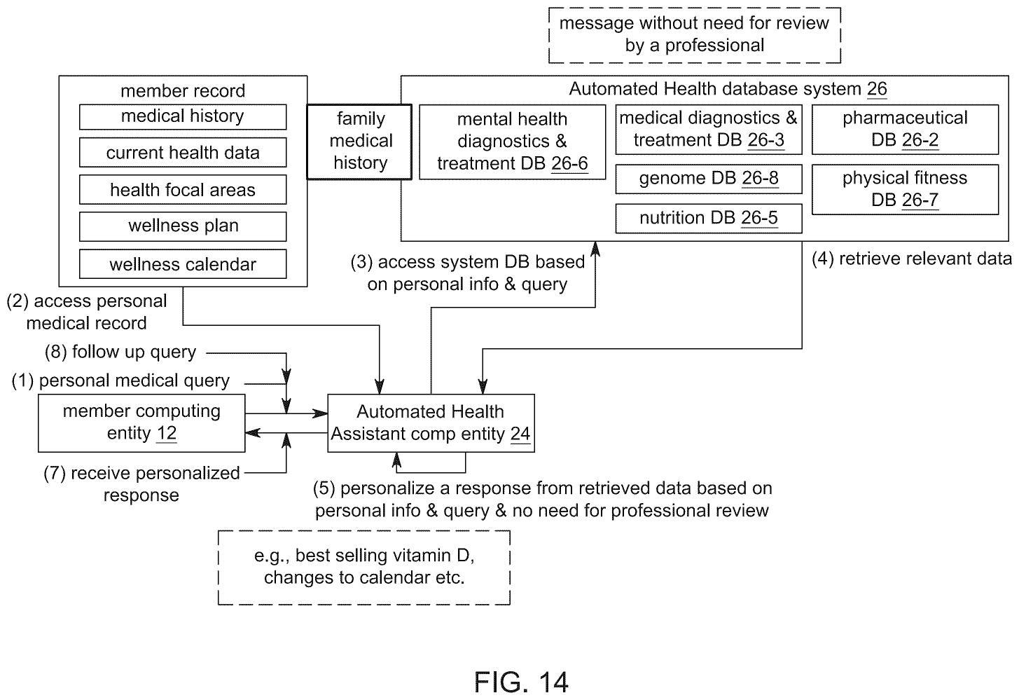

FIG. 13 is a schematic block diagram of another example of operation of computing system having an AI health assistant computing entity. In this example, the AI health assistant computing entity 24 facilitates, records, and AI augments a communication between a member, a professional and a pharmacist regarding a prescription refill and/or repurchase of supplements. FIG. 14 is a schematic block diagram of another example of operation of computing system having an AI health assistant computing entity. In this example, the AI health assistant computing entity 24 facilitates and AI augments a communication with a member regarding a personal medical query from a member.

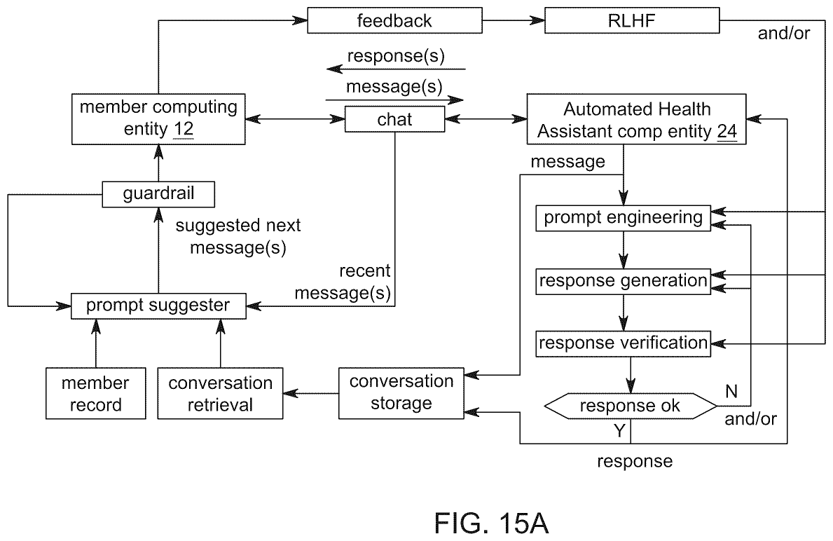

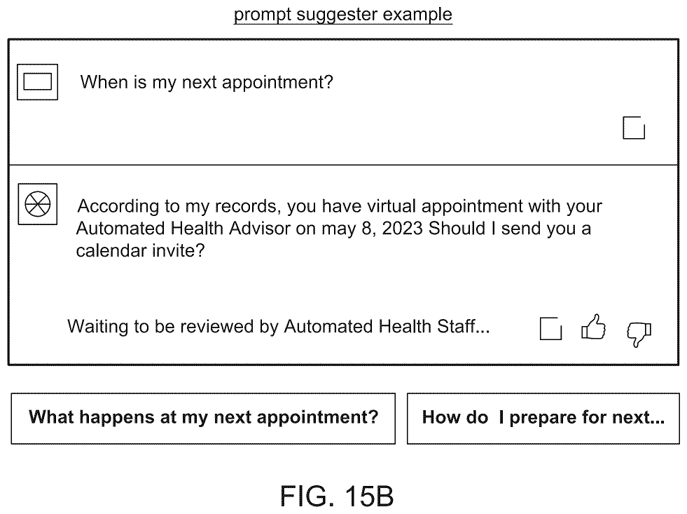

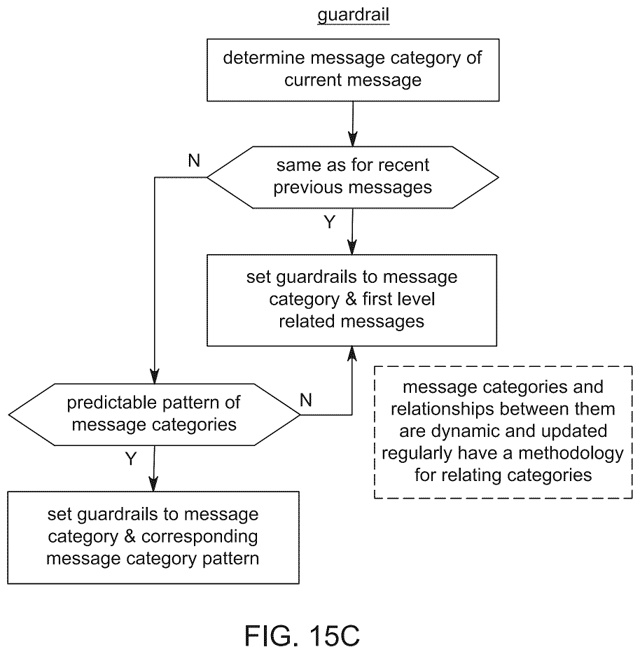

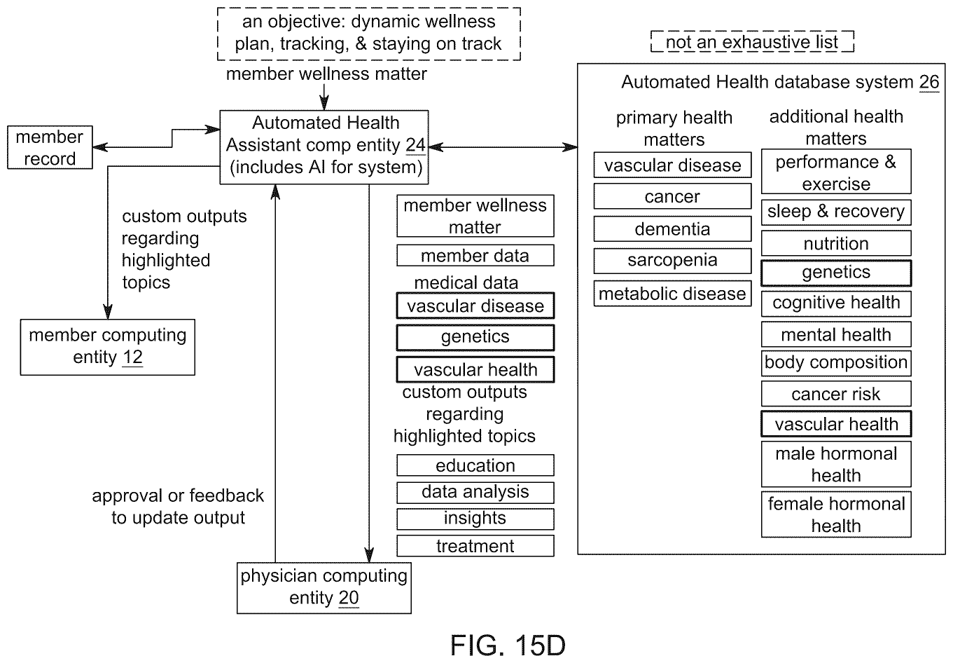

FIG. 15A is a schematic block diagram of another example of operation of computing system having an AI health assistant computing entity. In this example, the AI health assistant computing entity 24 facilitates, records, tracks, and AI augments a communication with a member. Prompt Guardrails ensure that a member's prompts are validated and filtered before being processed by the language model and helps maintain a safe and focused conversation by preventing the model from processing unsuitable inputs. For example, prompt guardrails may categorize a prompt to identify data set/model to query (e.g., is it a query about “my health data” or is it a question about my automated membership?).

Prompt engineering is a process of crafting effective input prompts for the language model, designed to elicit accurate and relevant responses. It includes defining the purpose and scope, using clear and concise language, and anticipating common user inputs. For example, prompt engineering may set context & rules (e.g., “You are a healthcare chatbot . . . ”), may include member meta-data and previous chats or transcripts with providers (e.g., “Patient has the following characteristics . . . ”), which “Facts” are relevant to the prompts, may include input restrictions (e.g., length, filter words), and may include output restrictions (e.g., format, temperature, max tokens, etc.)

Response verification involves checking the accuracy and reliability of the information provided by the language model. This can be achieved by integrating external fact-checking services, databases, or domain-specific knowledge sources. Alternatively, an ensemble of language models with different training data or architectures can be used to cross-verify generated content. For example, response verification may include the ability to score a response, fact-check a response, and filter a response for inappropriate words/phrases/advice.

Reinforcement Learning from Human Feedback (RLHF) is used to fine-tune the chatbot model by learning from user feedback. This involves collecting user ratings, explicit feedback, or implicit signals like conversation length to create a reward signal. This reward signal is then used to train a reinforcement learning model, which adjusts the chatbot's behavior to optimize for the desired outcomes.

Conversation storage is the infrastructure responsible for storing the chat history, user preferences, and other contextual information related to the conversation. It is used for maintaining context and ensuring a seamless user experience across sessions. The conversation storage component should also handle sensitive data securely, complying with privacy regulations like HIPAA or GDPR. For example, this may include storing all input and output and allow for the ability of members to view conversation history, use member conversations to train/test future chat architectures, and to show a track record over time of progress (or for regulatory reasons).

Regression Testing is an ability to try different 1) prompt architectures, and 2) models and assess how it impacted accuracy/speed against a predefined set of prompts/responses.

FIG. 15B is a schematic block diagram of another example of a communication prompt suggestion within a computing system having an AI health assistant computing entity. Here, the member asks when their next appointment is and the

FIG. 15C is a logic diagram of another example of a communication guardrail within a computing system having an AI health assistant computing entity. Here, for example, a process of determining a message category of a current message may begin by determining whether the category is the same as for recent previous messages. If so, the system may set guardrails to the message category and first level related messages. If no, then the system determines whether there is a predictable patter of message categories. If so, then guardrails to the message category and corresponding category pattern may be set. If not, the process may return to setting guardrails to the message category and first level related messages. In this way, it may be appreciated that message categories and relationships between them are dynamic and updated regularly and, further, that they have a methodology for relating categories.

FIG. 15D is a schematic block diagram of another example of operation of computing system having an AI health assistant computing entity. In this example, the AI health assistant computing entity 24 generates, with verification and/or updating by a professional, one or more custom outputs for a member based on a member wellness matter (e.g., lose 10 pounds), the member's personal medical data, and a multitude of medical data regarding the member wellness matter.

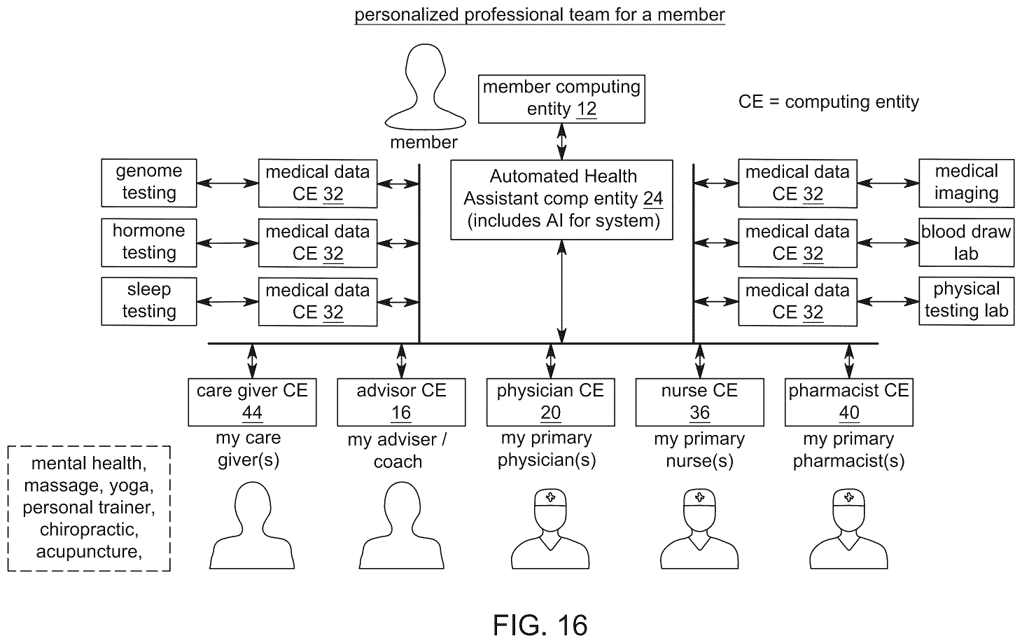

FIG. 16 is a schematic block diagram of an example of a team of supporting computing entities for a member of computing system having an AI health assistant computing entity. In this example, a member has a designated team of professionals. Here, member CE 12 may communicate with a personalized professional team vian automated health assistant CE 24 that includes AI-based system(s). Instances of medical data CE 32 may be associated with genome, hormone, and/or sleep testing. Similarly, medical data CEs 32 may also be associated with medical imaging, blood draw labs, and physical testing labs. This data is provided to care giver CE 44, advisor CE 16, physical CE 20, nurse CE 36, and pharmacist CE 40. These CEs may be associated with care givers, such as massage therapists, yoga teachers, personal trainers, chiropractors, and acupuncturists, as well as advisers or coaches, a primary care doctor, a primary nurse, and a primary pharmacist.

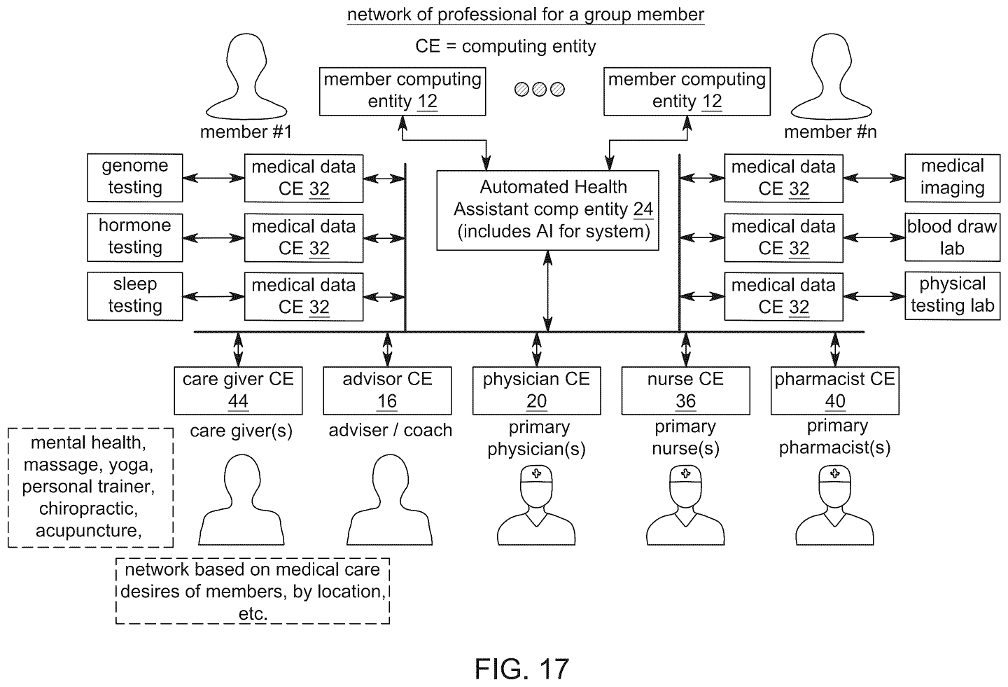

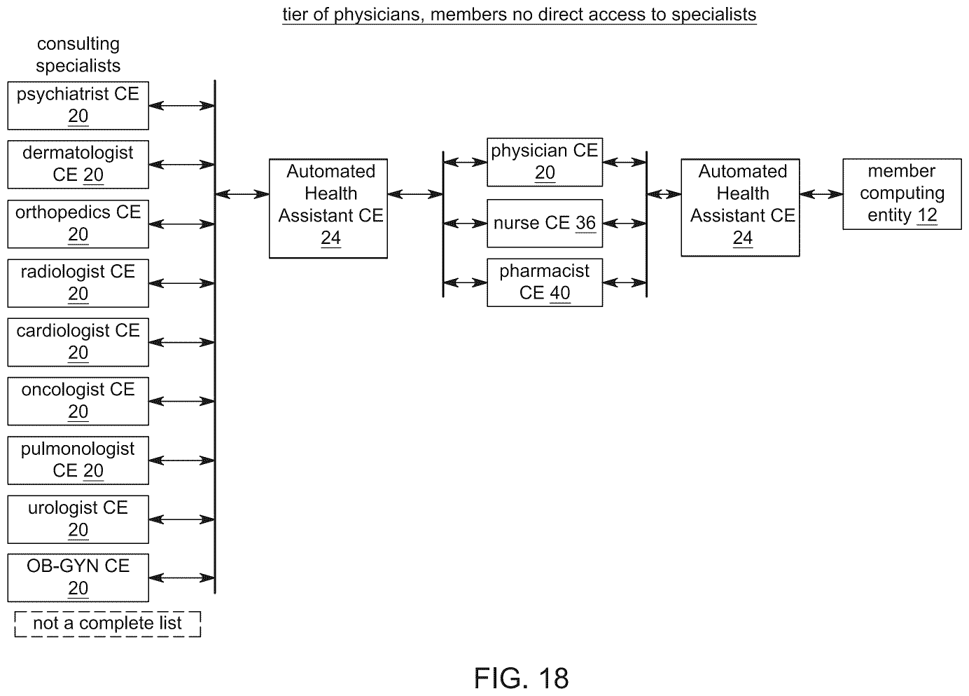

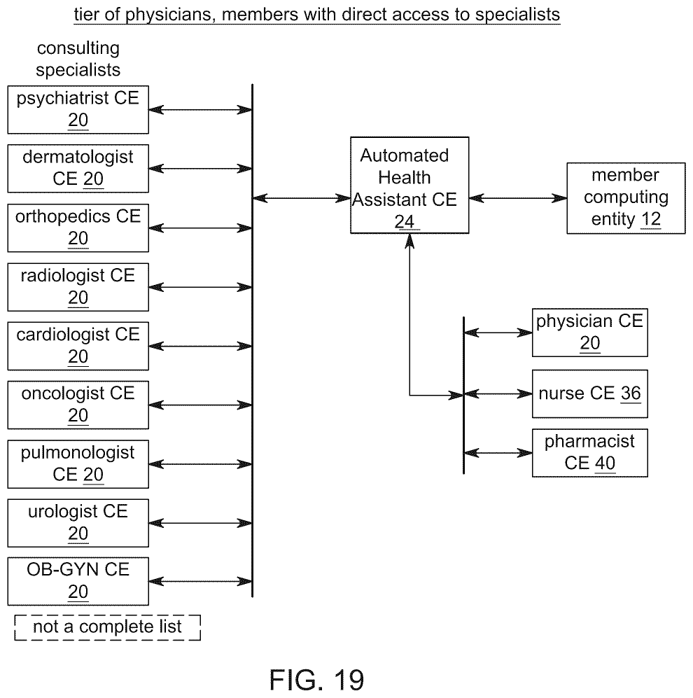

FIG. 17 is a schematic block diagram of another example of a team of supporting computing entities for members of computing system having an AI health assistant computing entity. In this example a team of professionals support a group of members.

FIG. 18 is a schematic block diagram of another example of a team of supporting computing entities for a member of computing system having an AI health assistant computing entity. In this example, professionals are tiered and not in direct contact with a member.

FIG. 19 is a schematic block diagram of another example of a team of supporting computing entities for a member of computing system having an AI health assistant computing entity. In this example, professionals are tiered and in direct contact with a member via the AI assistant computing entity 24.

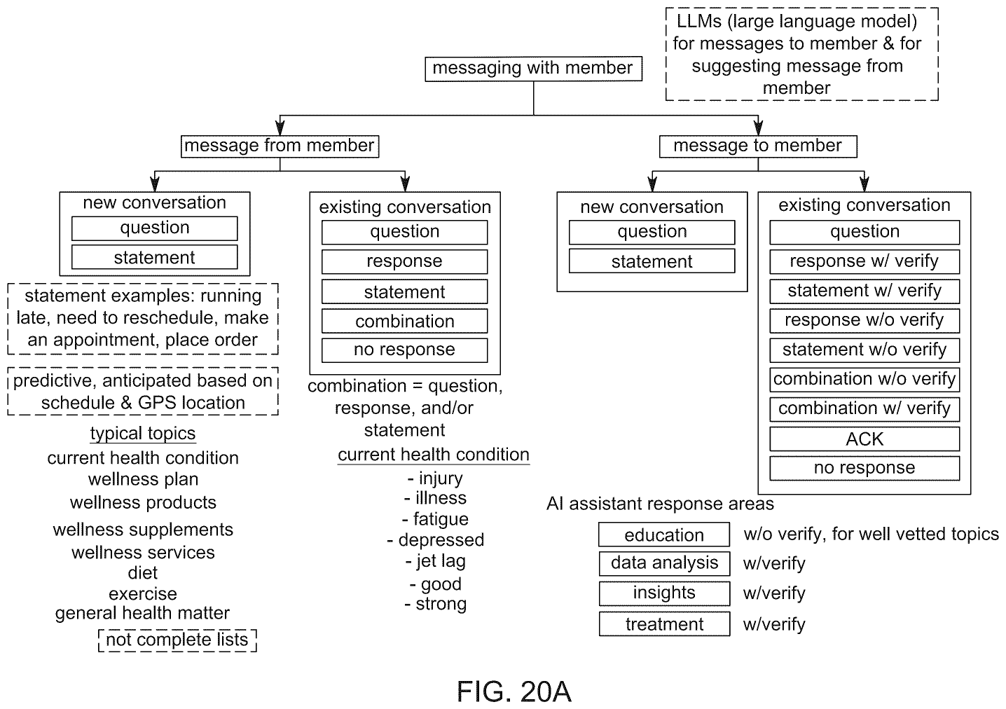

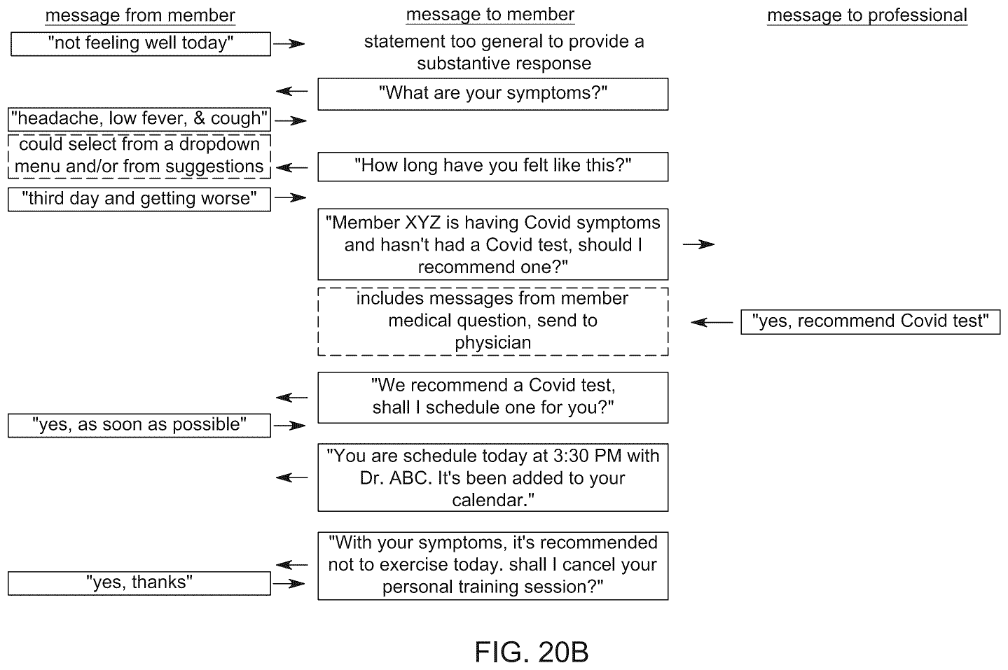

FIG. 20A is a schematic block diagram of another example of data communication and processing within a computing system having an AI health assistant computing entity. In this example, the AI assistant computing entity 24 categorizes messages with a member to facilitate AI augmenting and/or creating the messages to the member and/or predicting a next message from a member.

FIG. 20B is a schematic block diagram of another example of data communication and processing within a computing system having an AI health assistant computing entity. In this example, the AI assistant computing entity 24 facilitates and AI augments a communication between a member and the AI assistant with verification and/or input from a professional.

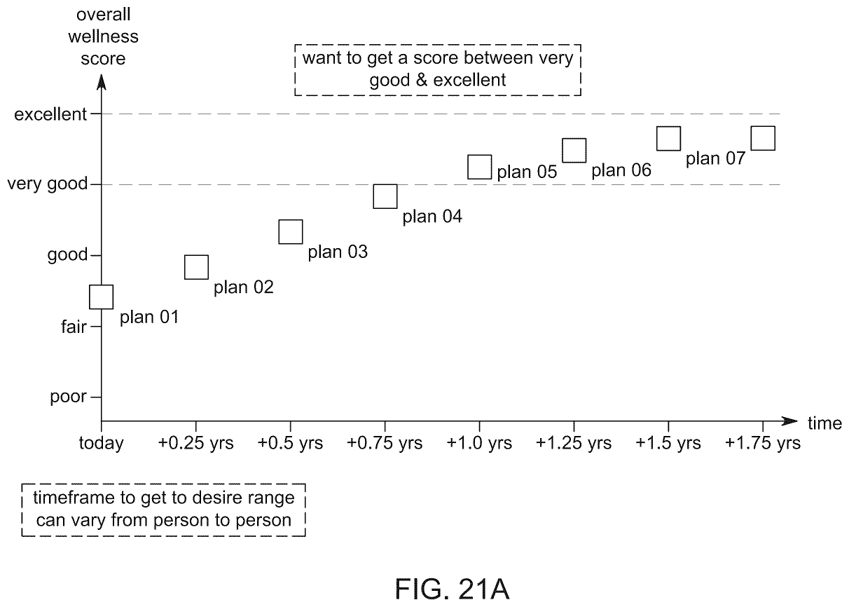

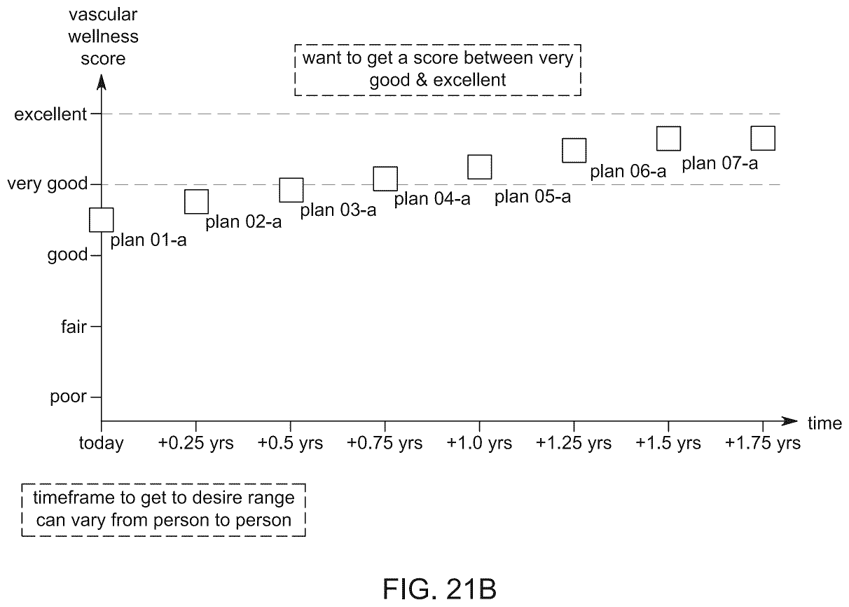

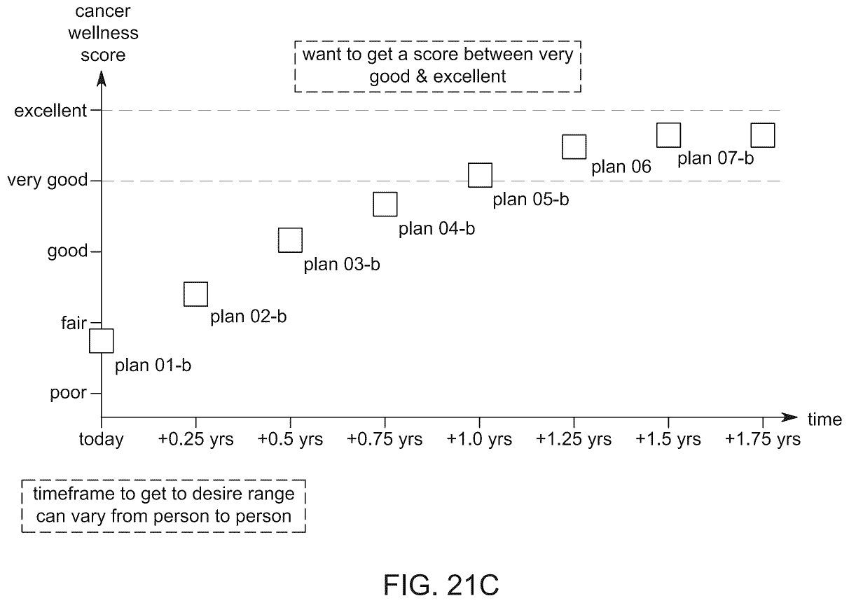

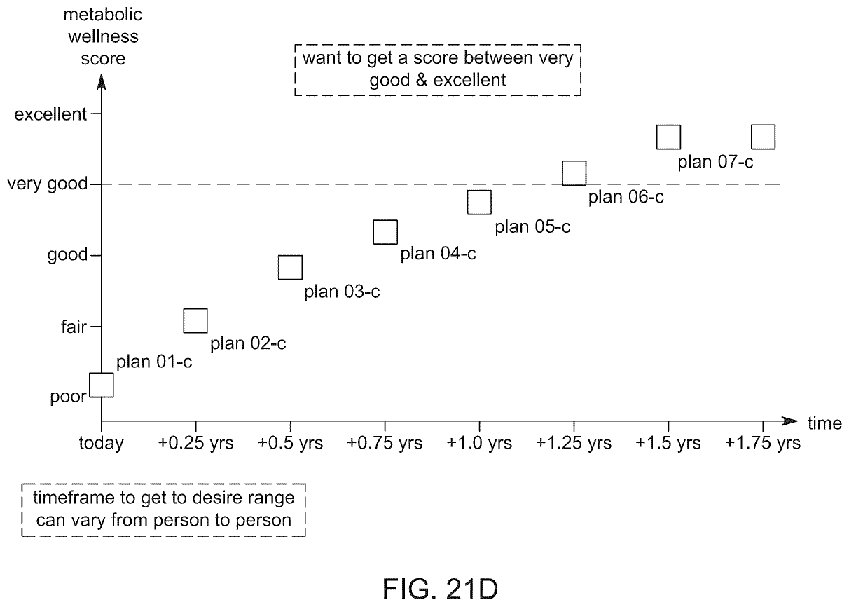

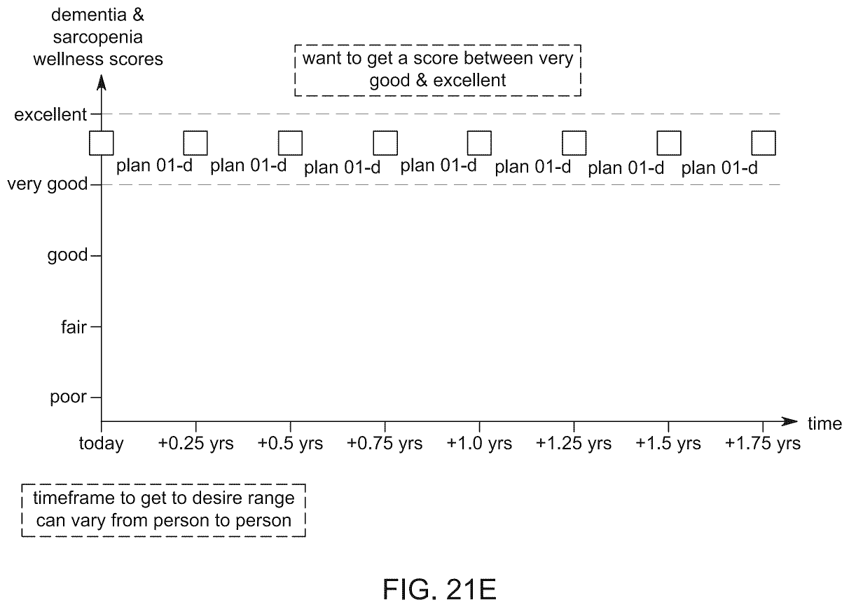

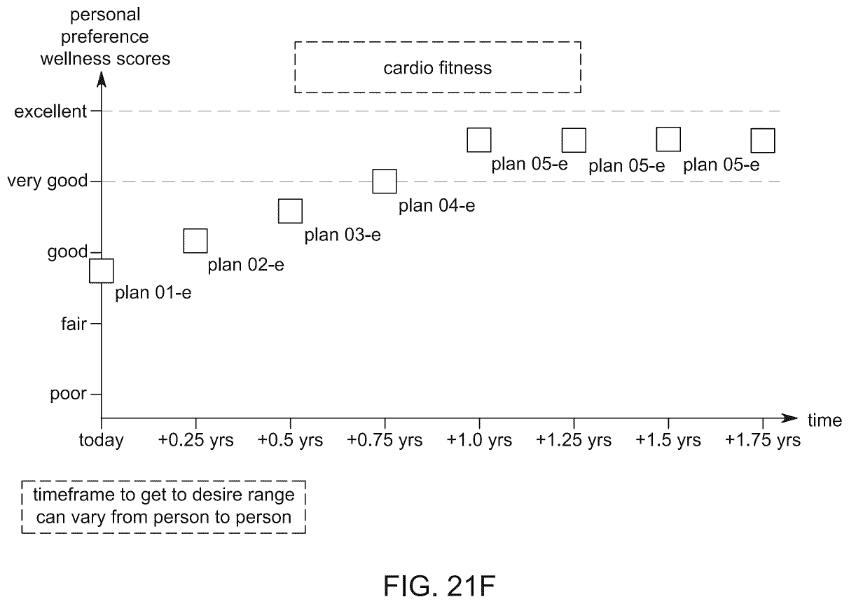

FIG. 21A is a schematic block diagram of an example of wellness plan graph as communicated and/or processed within a computing system having an AI health assistant computing entity. In this example, the graph is regarding overall wellness of the member, which is a weighted composite of individual wellness matter graphs.

FIG. 21B is a schematic block diagram of another example of wellness plan graph as communicated and/or processed within a computing system having an AI health assistant computing entity. In this example, the graph is regarding vascular wellness of the member, which is a weighted factor of the overall wellness graph.

FIG. 21C is a schematic block diagram of another example of wellness plan graph as communicated and/or processed within a computing system having an AI health assistant computing entity. In this example, the graph is regarding cancer wellness of the member, which is a weighted factor of the overall wellness graph. A poor score is indicative of personal health markers that the member is highly susceptible to contracting cancer in the near future.

FIG. 21D is a schematic block diagram of another example of wellness plan graph as communicated and/or processed within a computing system having an AI health assistant computing entity. In this example, the graph is regarding metabolic wellness of the member, which is a weighted factor of the overall wellness graph.

FIG. 21E is a schematic block diagram of another example of wellness plan graph as communicated and/or processed within a computing system having an AI health assistant computing entity. In this example, the graph is regarding dementia & sarcopenia wellness of the member, which is a weighted factor of the overall wellness graph.

FIG. 21F is a schematic block diagram of another example of wellness plan graph as communicated and/or processed within a computing system having an AI health assistant computing entity. In this example, the graph is regarding personal preference wellness (e.g., cardio fitness for running a marathon) of the member, which is a weighted factor of the overall wellness graph.

It may be appreciated from FIGS. 21A-21F that what score is considered very good or excellent and the timeframe to get to the desired range can vary from person to person. For example, achieving a vascular wellness score of very good may be quickly obtained in comparison to a cancer or metabolic wellness score.

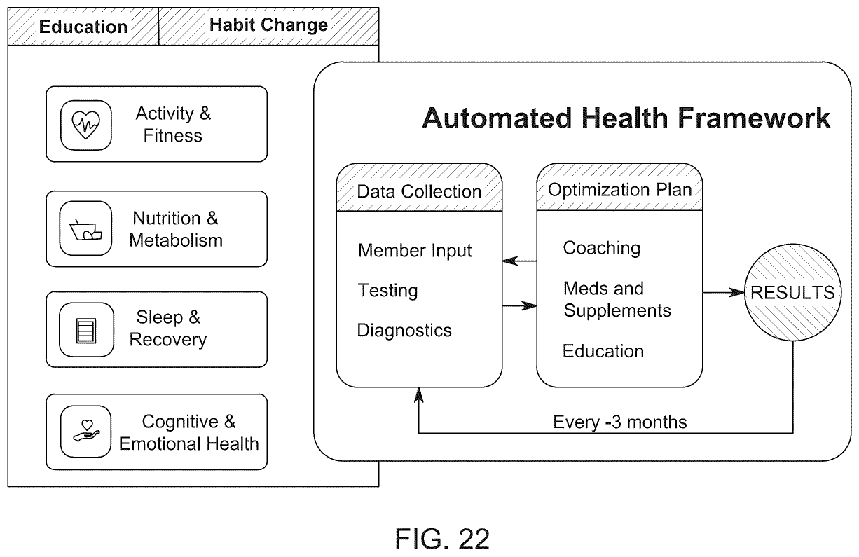

FIG. 22 is a schematic block diagram of an example of processing and/or communication of a wellness plan within a computing system having an AI health assistant computing entity.

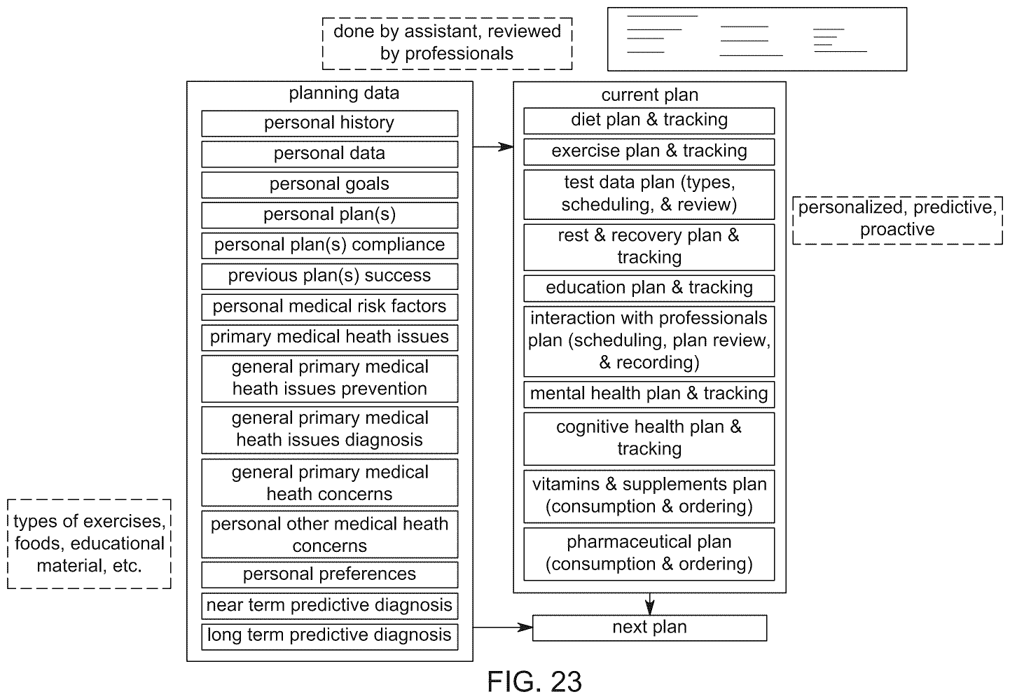

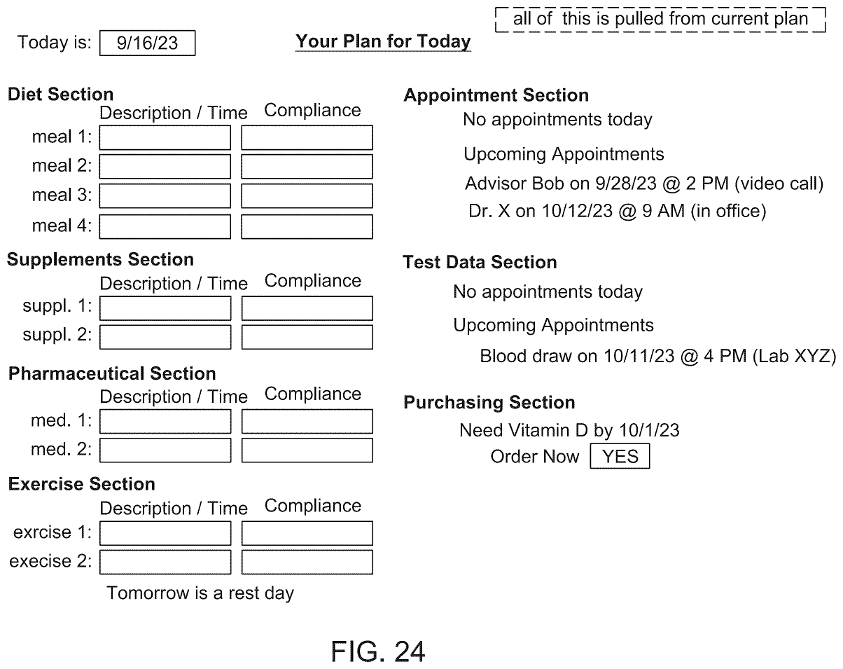

FIG. 23 is a schematic block diagram of another example of processing and/or communication of a wellness plan within a computing system having an AI health assistant computing entity. Here, planning data for developing a personalized plan for a member could include: personal history, personal data, personal goals, previous plan(s), compliance with previous plans, previous plan success, personal medical risk factors, primary medical health issues, general primary medical health issues prevention, general primary medical health issues diagnosis, general primary medical health concerns, personal other medical health concerns, personal preferences, near term predictive diagnosis, and long term predictive diagnosis.

This planning data is used to inform the member's current plan and a next plan. For example, a member's current plan may include personalized and proactive features such as: diet plan and tracking, exercise plan and tracking, test data plan, rest and recover plan and tracking, education plan and tracking, professional interactions plan, mental health plan and racking, vitamins and supplements plan, and pharmaceutical plan.

FIG. 24 is a schematic block diagram of another example of processing and/or communication of a day of a wellness plan within a computing system having an AI health assistant computing entity. Here, details pulled from the member's current plan may be presented via a UI that allows the member to quickly and easily view their meal times, medication times, exercises, as well as whether they have any upcoming appointments scheduled.

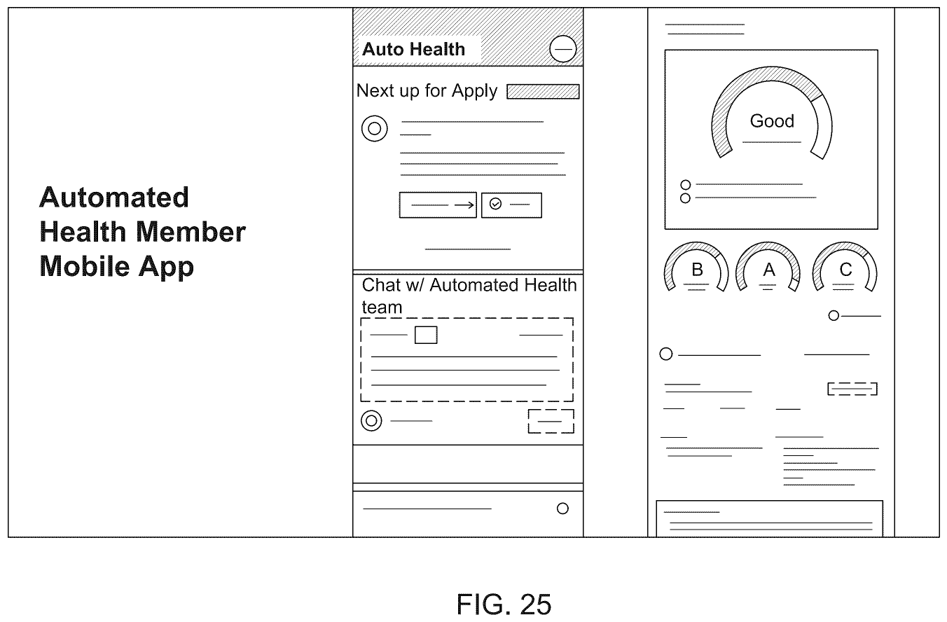

FIG. 25 is a schematic block diagram of another example of processing and/or communication information of a wellness plan within a computing system having an AI health assistant computing entity.

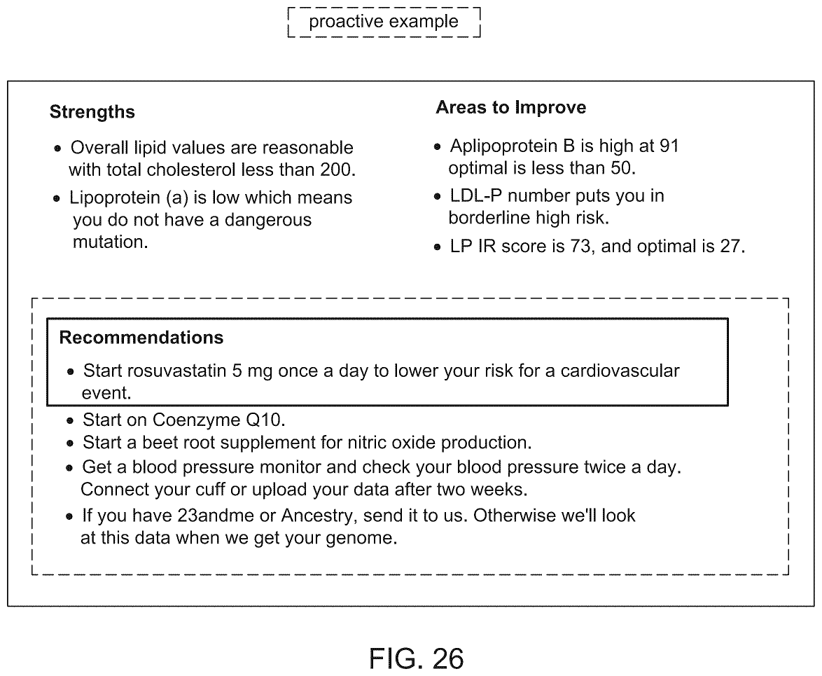

FIG. 26 is a schematic block diagram of another example of processing and/or communication information of a wellness plan within a computing system having an AI health assistant computing entity. Here, the recommendations produced by the system include practical and specific actions such as starting rosuvastatin 5 mg once a day. It is appreciated that this recommendation is personalized to each member at a given point in time and may include both a specific medication and dosage, but also an interval or schedule for taking the recommended medication.

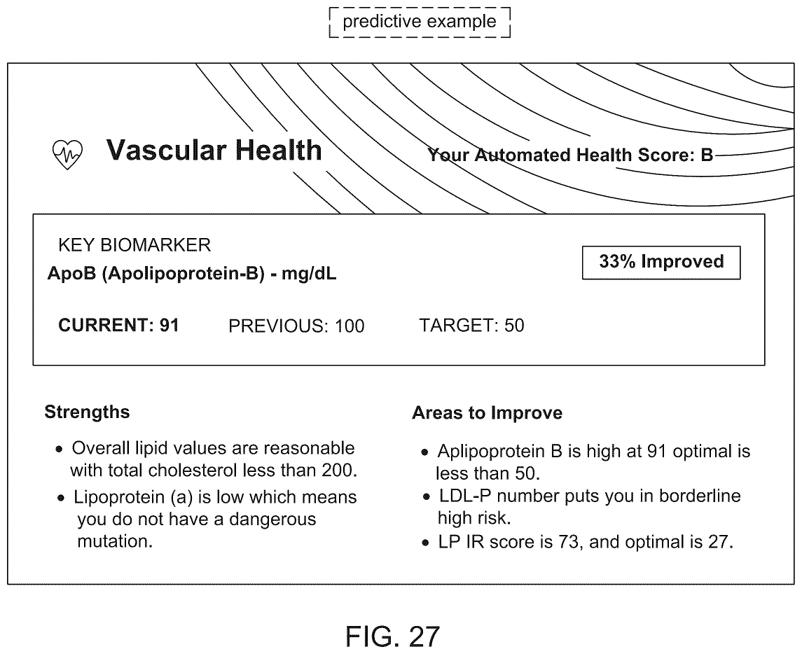

FIG. 27 is a schematic block diagram of another example of processing and/or communication information of a wellness plan within a computing system having an AI health assistant computing entity. Here, the member's vascular health can be summarized as a score between 1-100. This value may be tracked over time, such as showing a current vascular health score of 91 and a previous vascular health score of 100, representing a 33% improvement. Similarly, the member's overall health score (Automated Health Score) may be represented as a report card such as A, B, C, D, and F. In this case, the member's overall score is a B, representing the second highest score category.

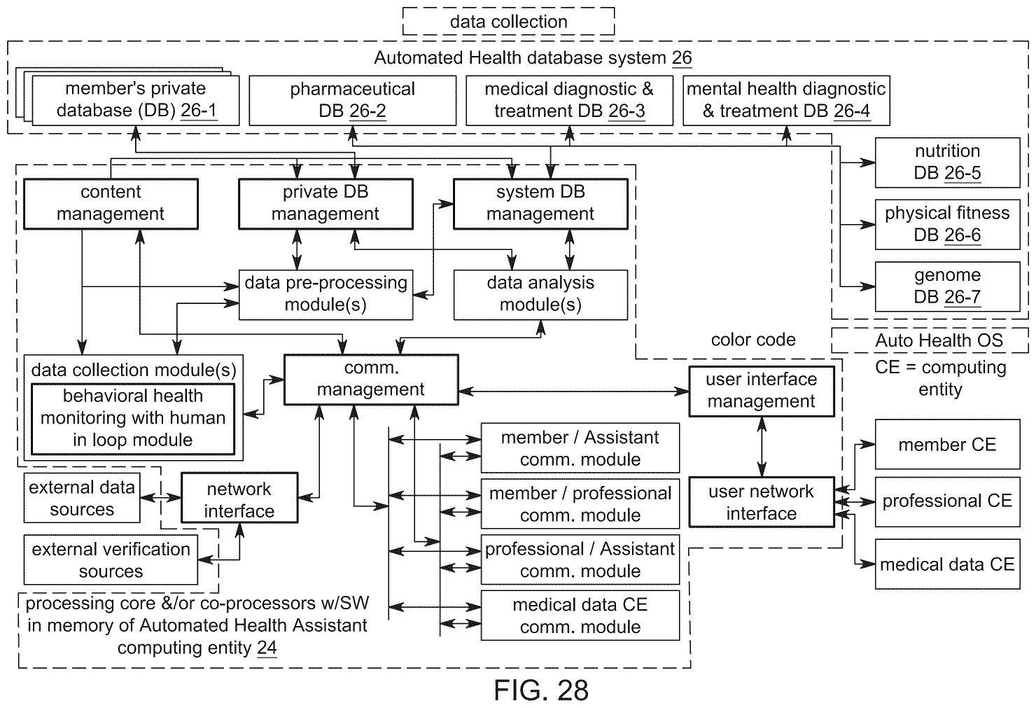

FIG. 28 is a schematic block diagram of an embodiment of computing system having an AI health assistant computing entity that processes behavioral health monitoring data. In this example, the behavioral health monitoring with human-in-the-loop utilizes algorithms, voice intonation, and text analytics to assess mental health, with a human in the loop for review and interventions as needed.

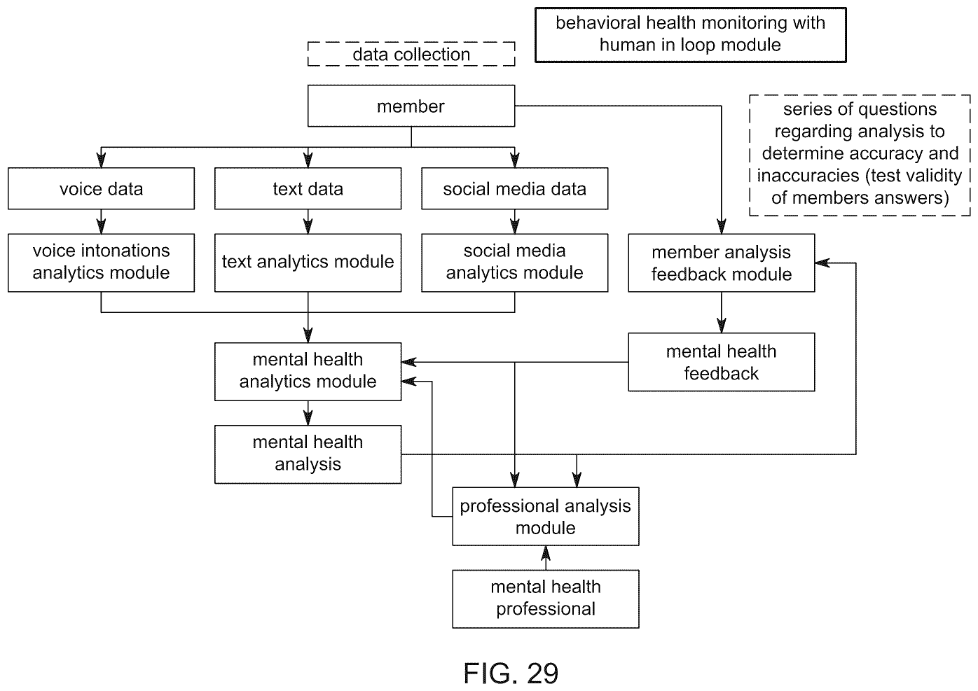

FIG. 29 is a schematic block diagram of another embodiment of computing system having an AI health assistant computing entity that processes behavioral health monitoring data.

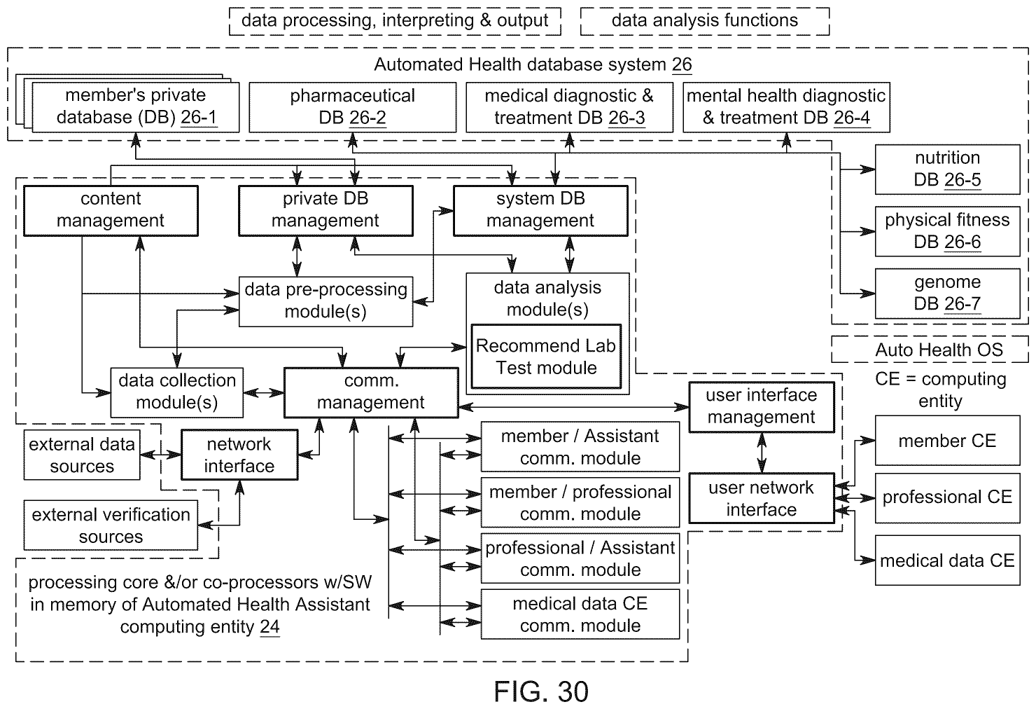

FIG. 30 is a schematic block diagram of an embodiment of computing system having an AI health assistant computing entity that processes lab test recommendation data. In this example, the AI-driven lab test recommendation module provides personalized lab test suggestions based on medical history, recent interactions, remote health monitoring, and wearable biomarker tracking like Continuous Glucose Monitoring (CGM) data.

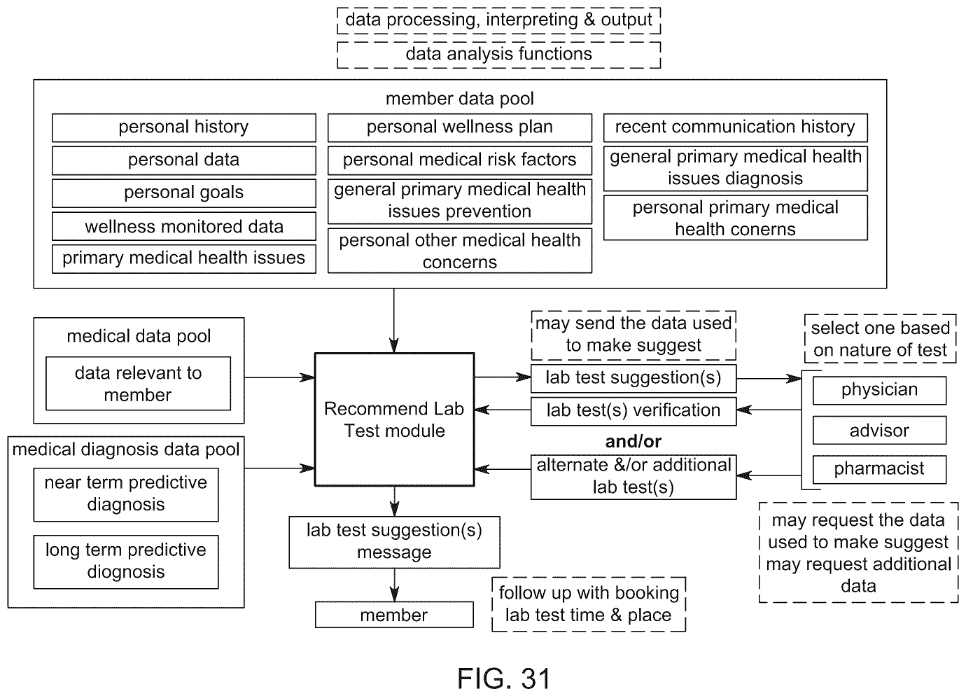

FIG. 31 is a schematic block diagram of another embodiment of computing system having an AI health assistant computing entity that processes lab test recommendation data.

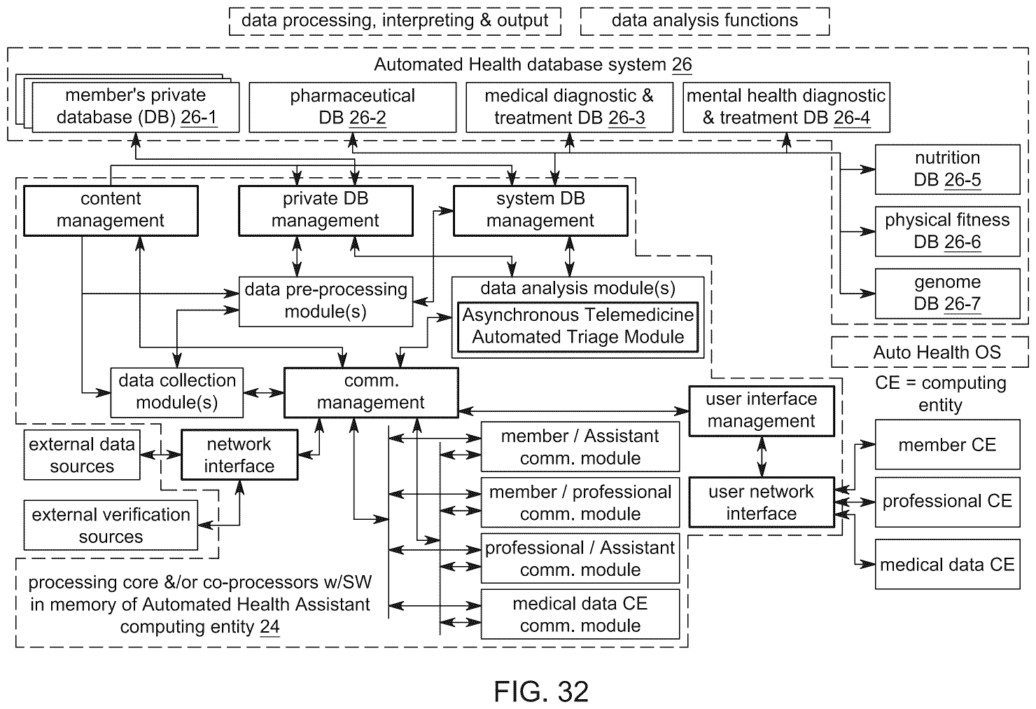

FIG. 32 is a schematic block diagram of an embodiment of computing system having an AI health assistant computing entity that processes asynchronous telemedicine triage data. In this example, the Asynchronous Telemedicine Automated Triage Algorithm categorizes patient queries based on urgency, the type of team member needed (e.g., nurse, pharmacist), the medical knowledge required to answer the question, and the specific medical data needed from the patient's chart.

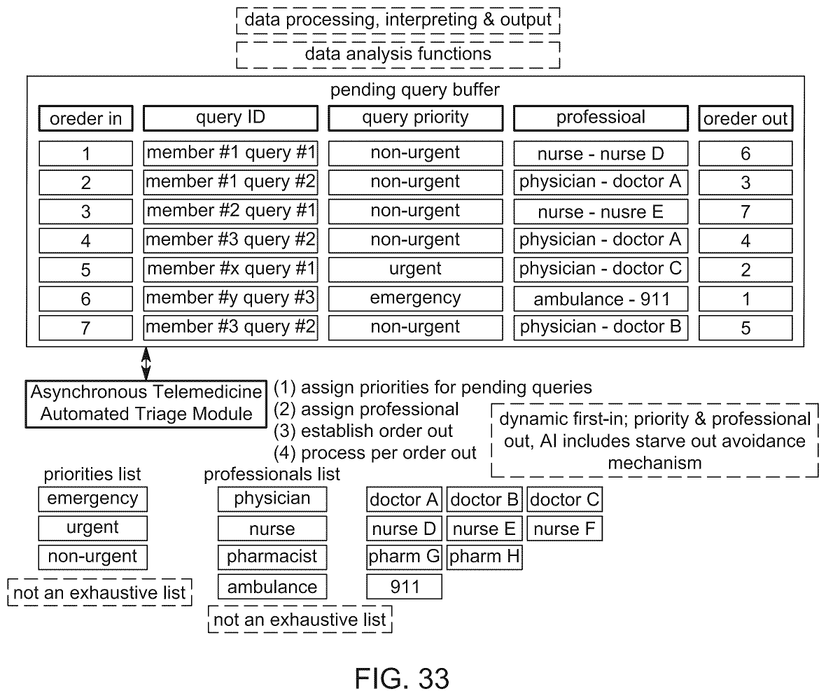

FIG. 33 is a schematic block diagram of another embodiment of computing system having an AI health assistant computing entity that processes asynchronous telemedicine triage data.

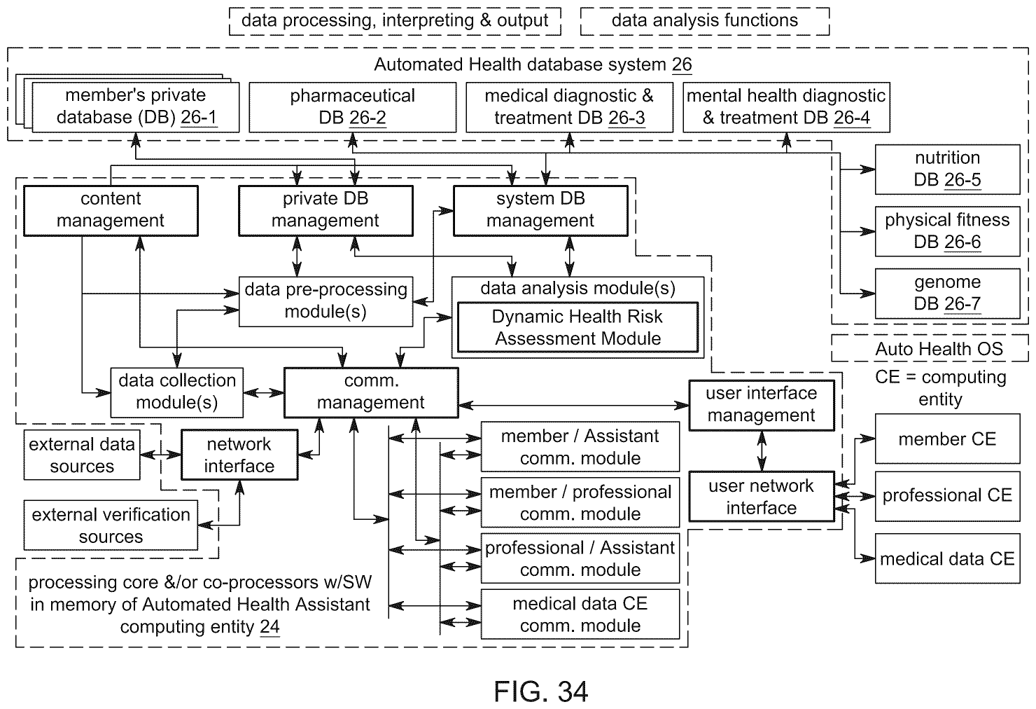

FIG. 34 is a schematic block diagram of an embodiment of computing system having an AI health assistant computing entity that processes health risk assessment data. In this example, the Dynamic Health Risk Assessment regularly updates health risk profiles of individuals based on a range of factors including biomarkers, whole genome sequencing, lifestyle, wearable data, imaging studies, and new research.

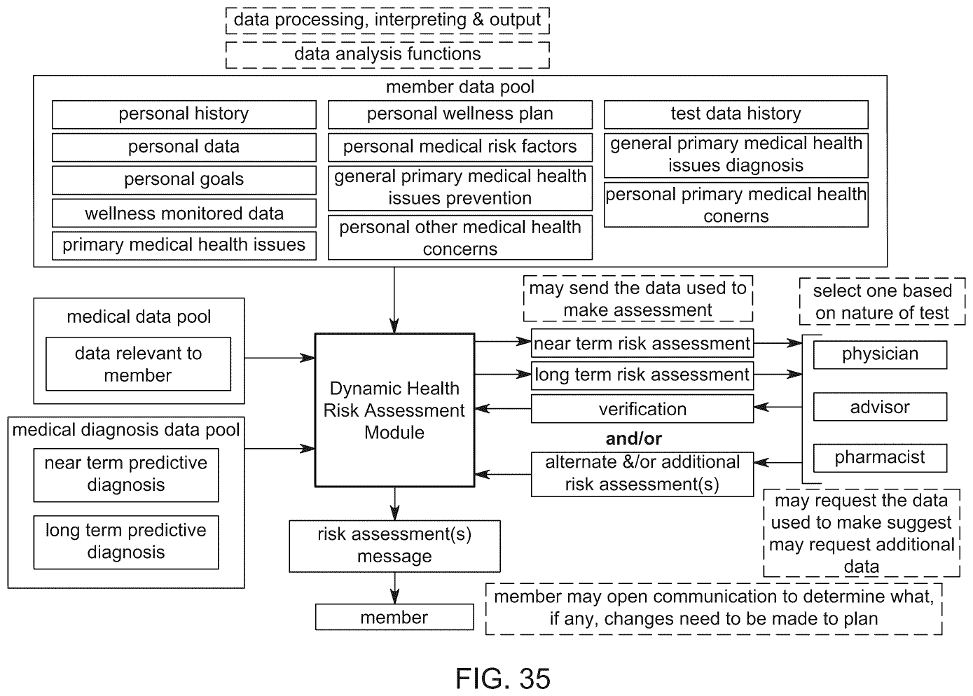

FIG. 35 is a schematic block diagram of another embodiment of computing system having an AI health assistant computing entity that processes health risk assessment data.

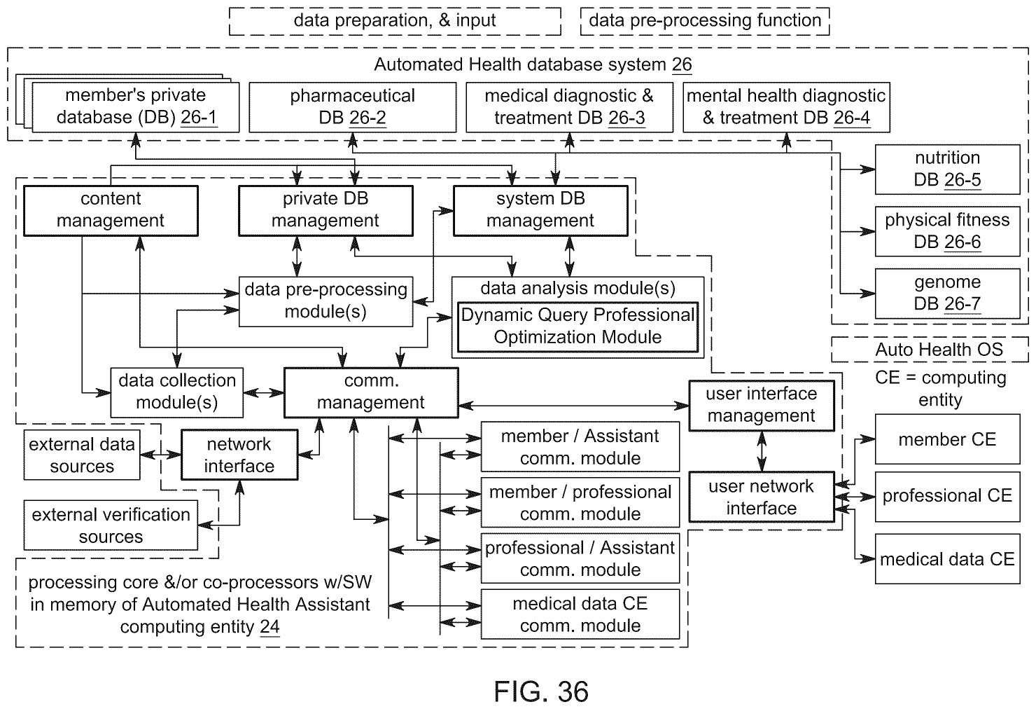

FIG. 36 is a schematic block diagram of an embodiment of computing system having an AI health assistant computing entity that processes query of a professional data. In this example, the Dynamic Query Optimization for Team Members adjusts the medical references based on the complexity of the patient's query and the expertise of the team member reviewing it. For example, nursing references for nurses, pharmacy knowledge for pharmacists, etc.

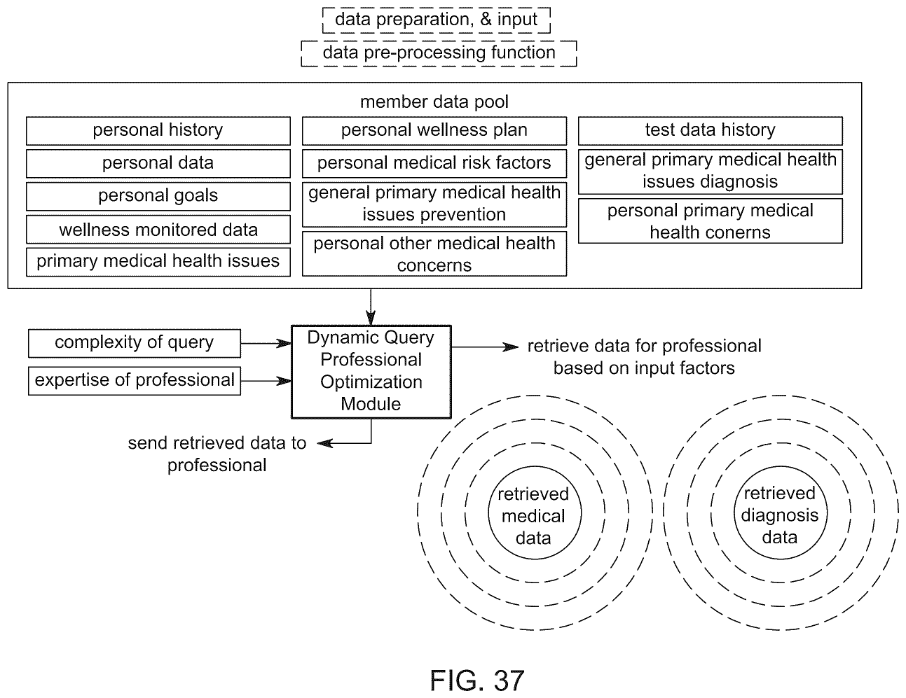

FIG. 37 is a schematic block diagram of another embodiment of computing system having an AI health assistant computing entity that processes query of a professional data.

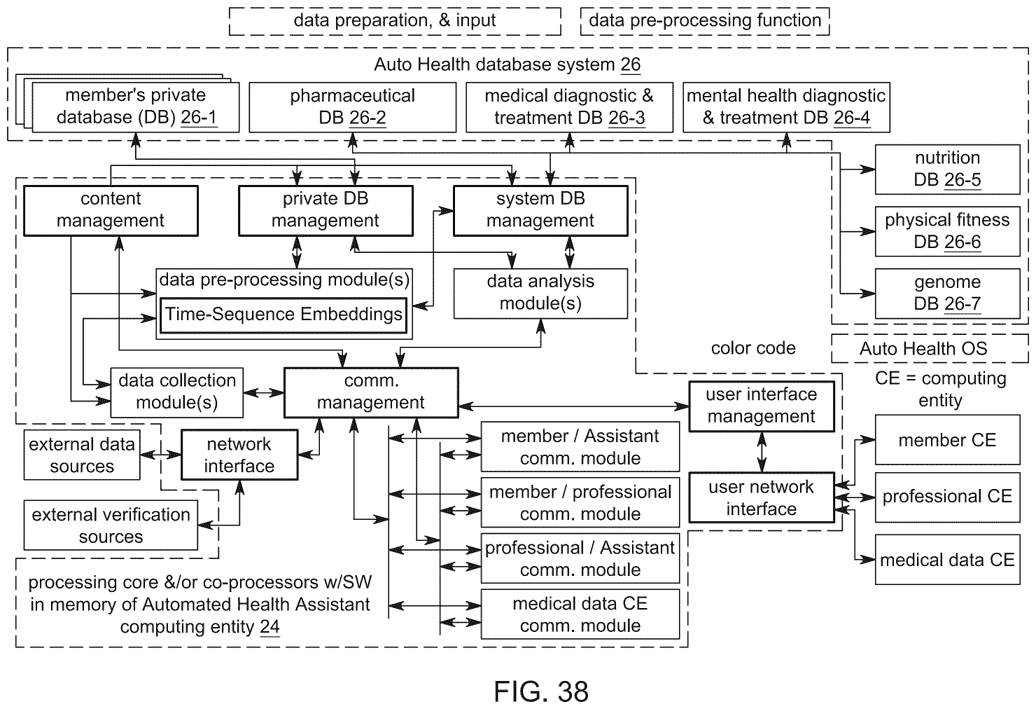

FIG. 38 is a schematic block diagram of an embodiment of computing system having an AI health assistant computing entity that processes time sequence embedding data. In this example, the Time-Sequence Embeddings captures the temporal relationships in medical data for improved predictive analysis, providing a granular view of health trajectories and facilitating more accurate predictive modeling.

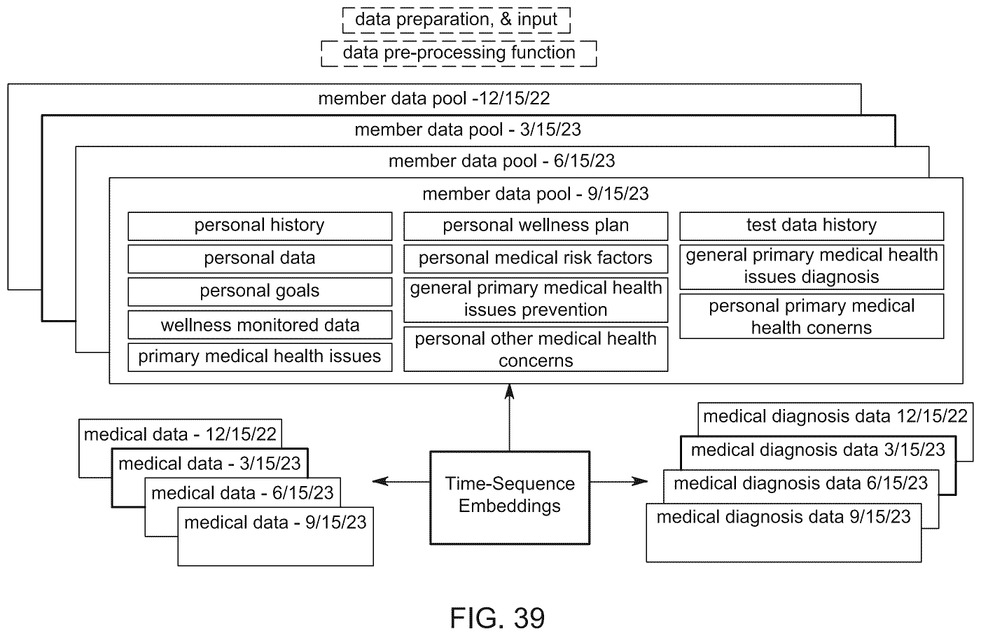

FIG. 39 is a schematic block diagram of another embodiment of computing system having an AI health assistant computing entity that processes time sequence embedding data.

Processing time sequence embeddings may be an important part of processing personal medical data that evolves over time. Processing time sequence embeddings is important for several reasons. First, it helps in capturing the temporal dynamics of medical conditions, which is crucial for understanding how diseases progress over time and how patients respond to treatments. This process enables the detection of patterns that may not be obvious from isolated data snapshots, revealing subtle trends in vital signs and early warning signs of complications.

The process may begin by gathering temporal medical data such as patient records, vital signs, symptom logs, and treatment histories, all of which include timestamps to indicate when each entry was recorded. The next step is to preprocess this data to ensure consistency, which includes handling missing values, standardizing formats, and transforming raw data into a suitable format for embedding.

Once the data is prepared, it may be organized into time sequence representations. This involves defining time windows or intervals for analysis, such as daily or monthly, depending on the level of detail required. The data are then arranged into sequences based on these intervals, creating a series of events or measurements over time for each patient.

The next stage may include embedding generation where relevant features are extracted from the temporal data. These features might include physiological measurements, symptoms, and other health indicators, which are then converted into vector representations known as embeddings. Techniques such as Recurrent Neural Networks (RNNs), Long Short-Term Memory networks (LSTMs), or Transformer-based models are used to capture temporal dependencies and relationships within the data.

Temporal context analysis may be performed next, where contextual information like patient demographics or treatment history is integrated into the embeddings to enrich the model's understanding. This helps in analyzing trends, patterns, and anomalies in the patient's medical data over time, which could involve detecting changes in health status, predicting future health events, or identifying correlations between different health indicators.

The final steps may include model training and evaluation. Predictive models are trained using the time sequence embeddings to make forecasts or classifications, such as predicting disease progression or response to treatment. The performance of these models is assessed using various metrics and the models are validated with real-world data to ensure their effectiveness.

Additionally, time sequence embeddings enhance predictive accuracy by capturing the temporal relationships in medical data. This leads to more accurate predictions of patient outcomes, disease progression, and treatment efficacy. They also support personalized medicine by providing insights into how individual patients' conditions evolve, allowing for tailored interventions.

Early intervention is another key benefit. By identifying anomalies or deviations from normal patterns, time sequence embeddings enable timely interventions that could prevent serious health issues. They support proactive health management by predicting future health events and recommending preventive measures.

Finally, time sequence embeddings facilitate informed decision-making. They provide valuable insights for healthcare providers, aiding in clinical decisions by offering a comprehensive view of a patient's health over time. This data-driven approach enhances decision-making and helps in developing effective treatment plans based on detailed analysis of trends and correlations.

In summary, processing time sequence embeddings involves organizing temporal medical data into sequential formats, generating vector representations, and analyzing these embeddings to capture patterns and trends. This process is essential for understanding disease progression, improving predictive accuracy, enabling early intervention, and supporting informed healthcare decisions.

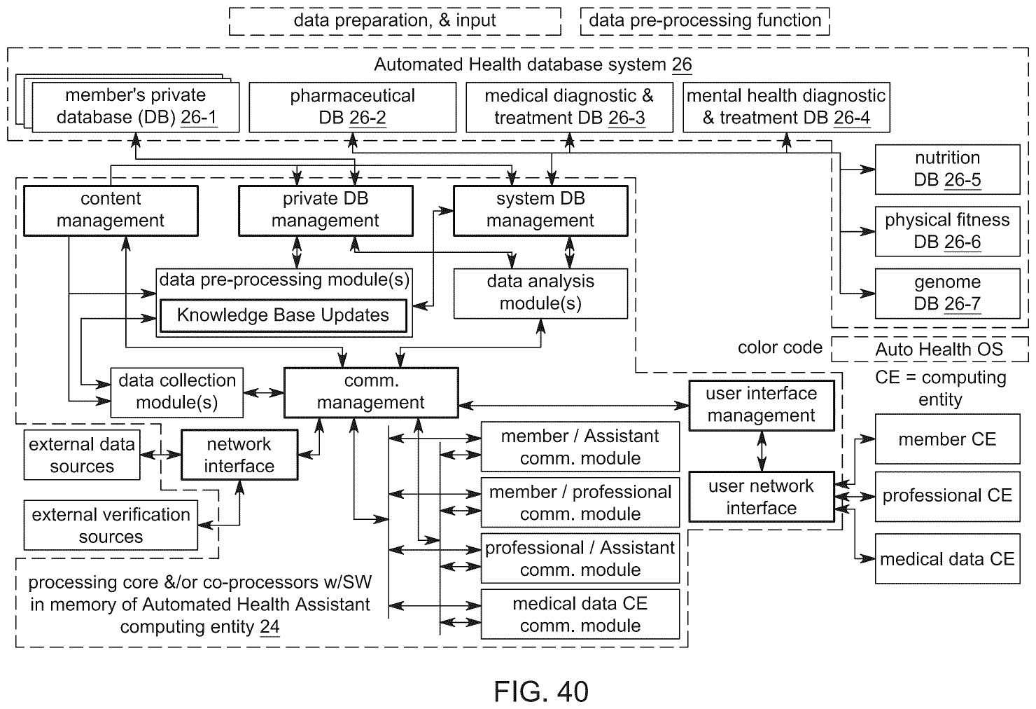

FIG. 40 is a schematic block diagram of an embodiment of computing system having an AI health assistant computing entity that processes updating of knowledge base data. In this example, the Incremental Knowledgebase Update Mechanism enables real-time or near-real-time updates of the knowledgebase using AI agents, ensuring that the database remains current and relevant.

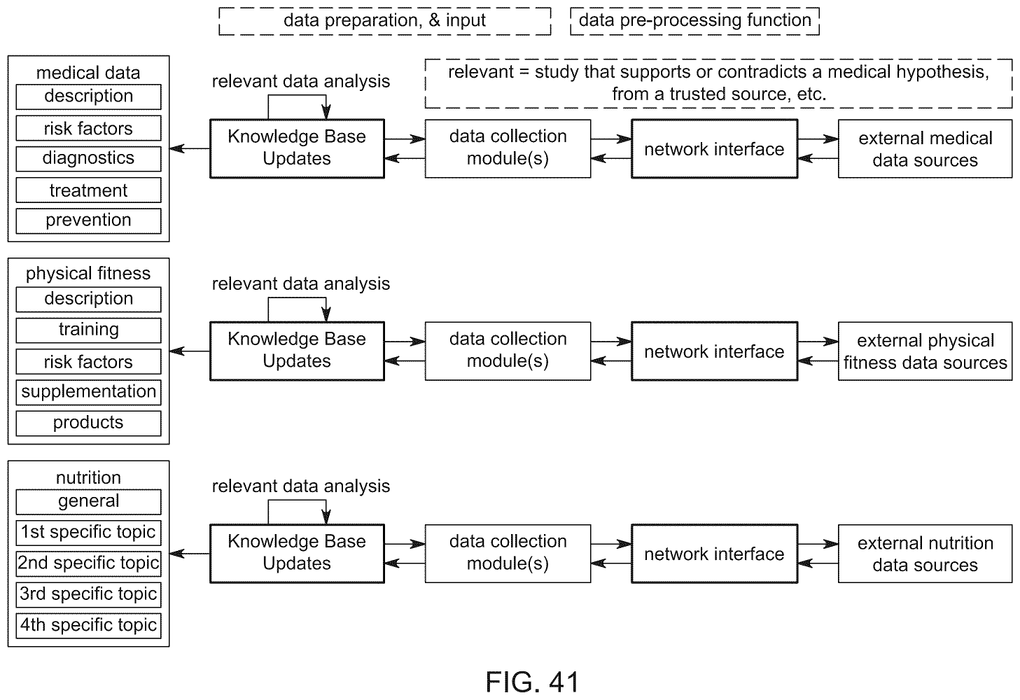

FIG. 41 is a schematic block diagram of another embodiment of computing system having an AI health assistant computing entity that processes updating of knowledge base data.

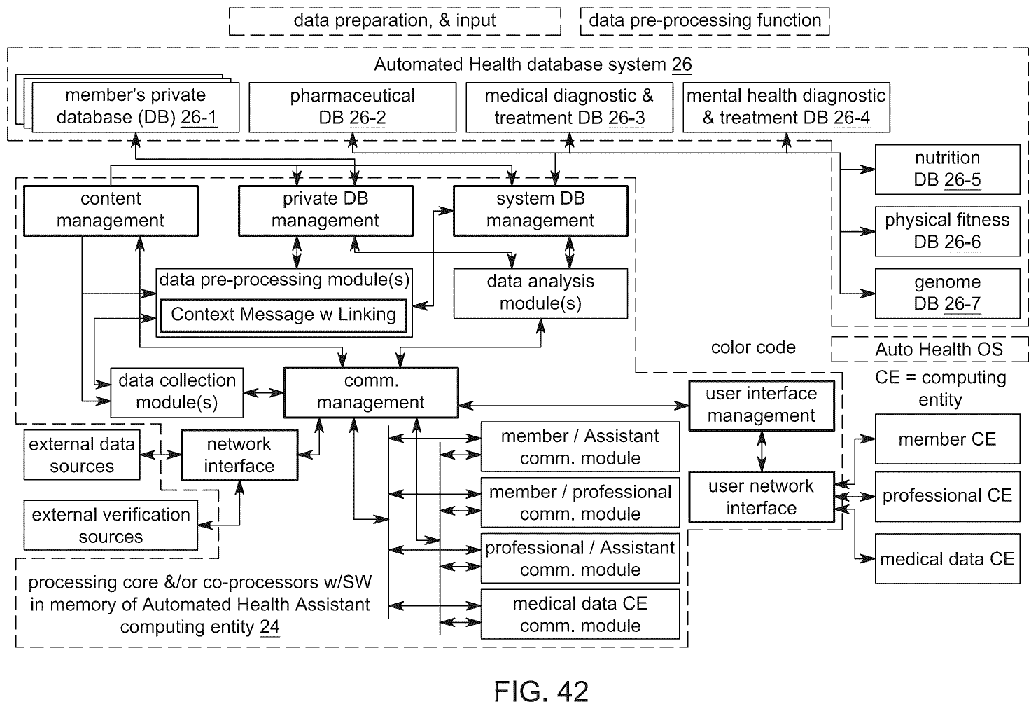

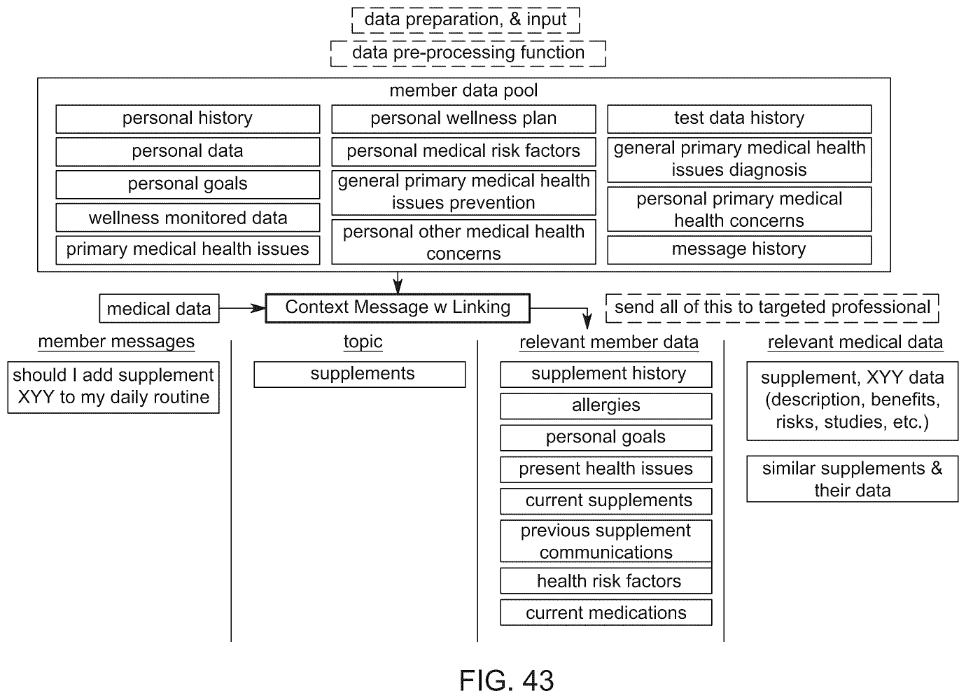

FIG. 42 is a schematic block diagram of an embodiment of computing system having an AI health assistant computing entity that processes linking of messages with background data. In this example, the Context-Aware Messaging with Deep Linking provides relevant medical history alongside patient messages and contains deep links into a patient's medical record so that a clinician, nurse, pharmacist, health coach can easily verify data and medical sources presented by an AI assistant.

FIG. 43 is a schematic block diagram of another embodiment of computing system having an AI health assistant computing entity that processes linking of messages with background data.

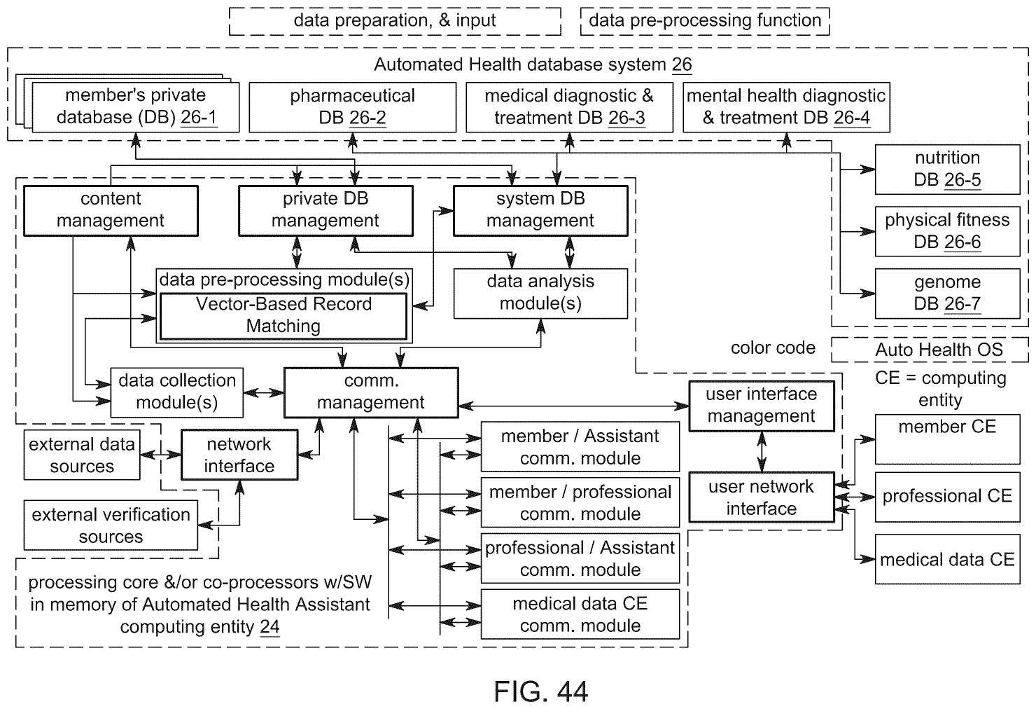

FIG. 44 is a schematic block diagram of an embodiment of computing system having
an AI health assistant computing entity that processes vector-based record matching data. In this example, the Vector-Based Record Matching with Embeddings links fragmented medical records across databases using vector similarity and normalizes data into a centralized, portable medical record for the member. The key feature is the ingestion and vectorized nature of the database behind it.

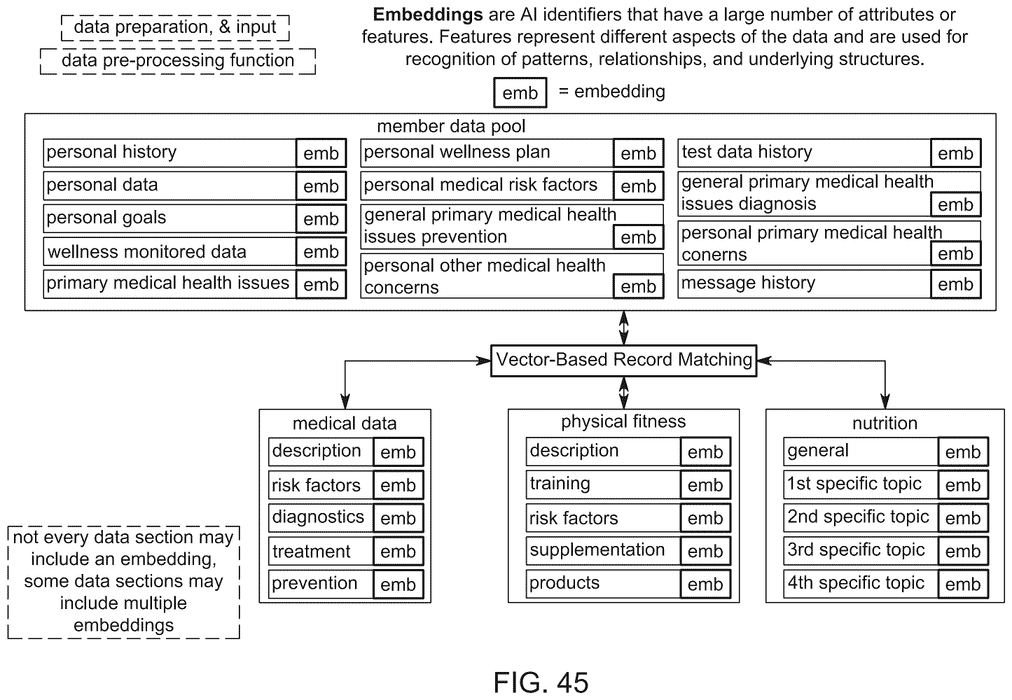

FIG. 45 is a schematic block diagram of another embodiment of computing system having an AI health assistant computing entity that processes vector-based record matching data.

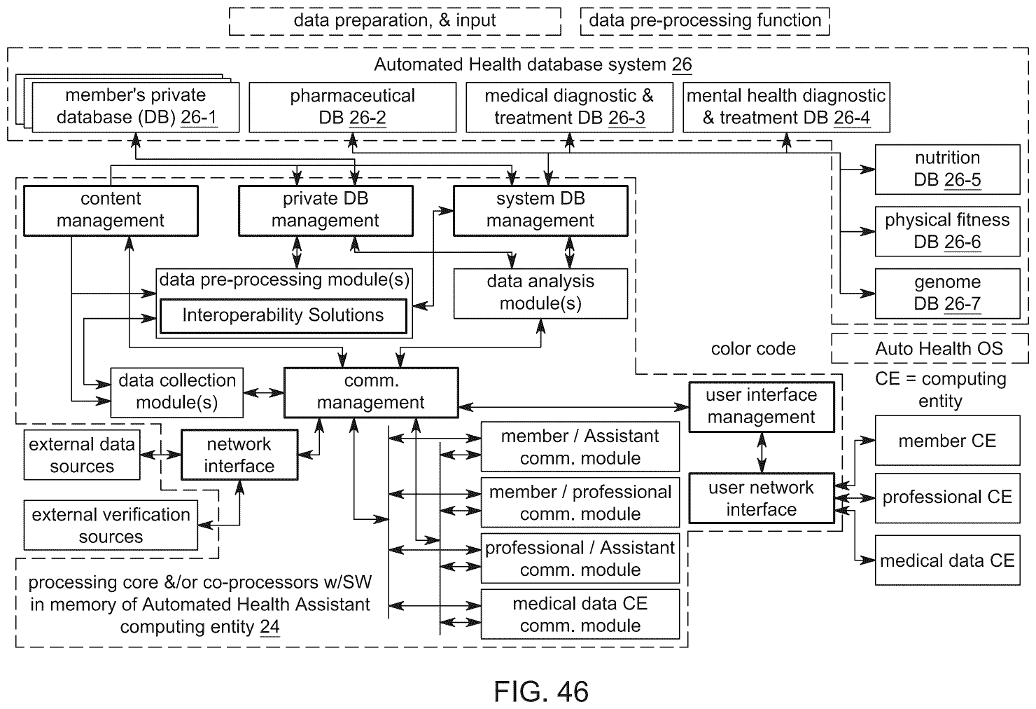

FIG. 46 is a schematic block diagram of an embodiment of computing system having an AI health assistant computing entity that processes interoperability of data. In this example, the Interoperability Solutions converts various data formats from HIEs into a standardized, searchable format, solving a significant pain point in healthcare IT. Accomplished using a vectorized database with embeddings.

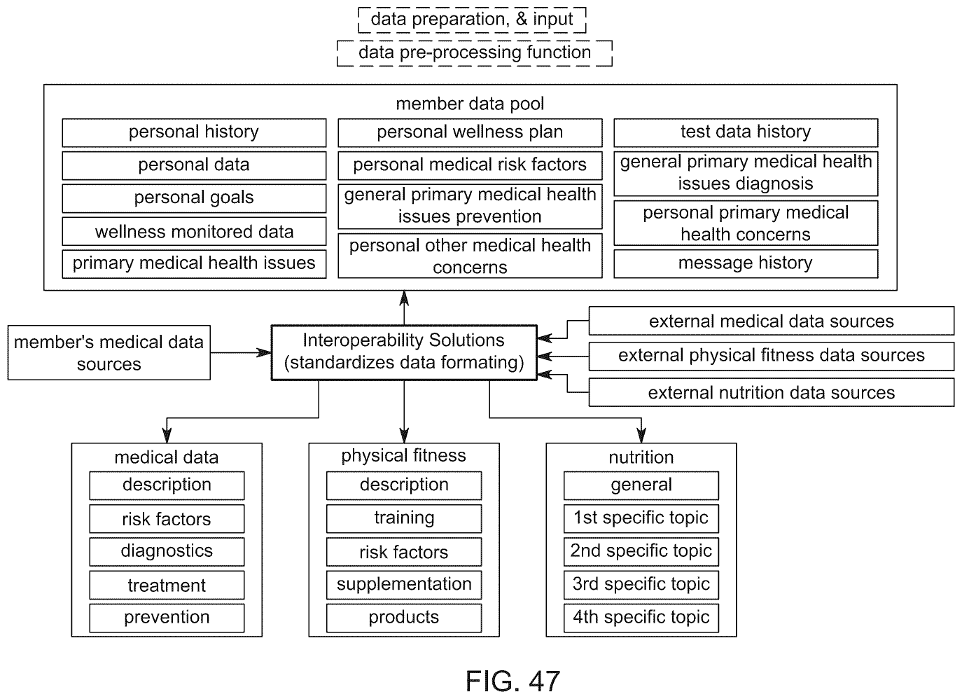

FIG. 47 is a schematic block diagram of another embodiment of computing system having an AI health assistant computing entity that processes interoperability of data.

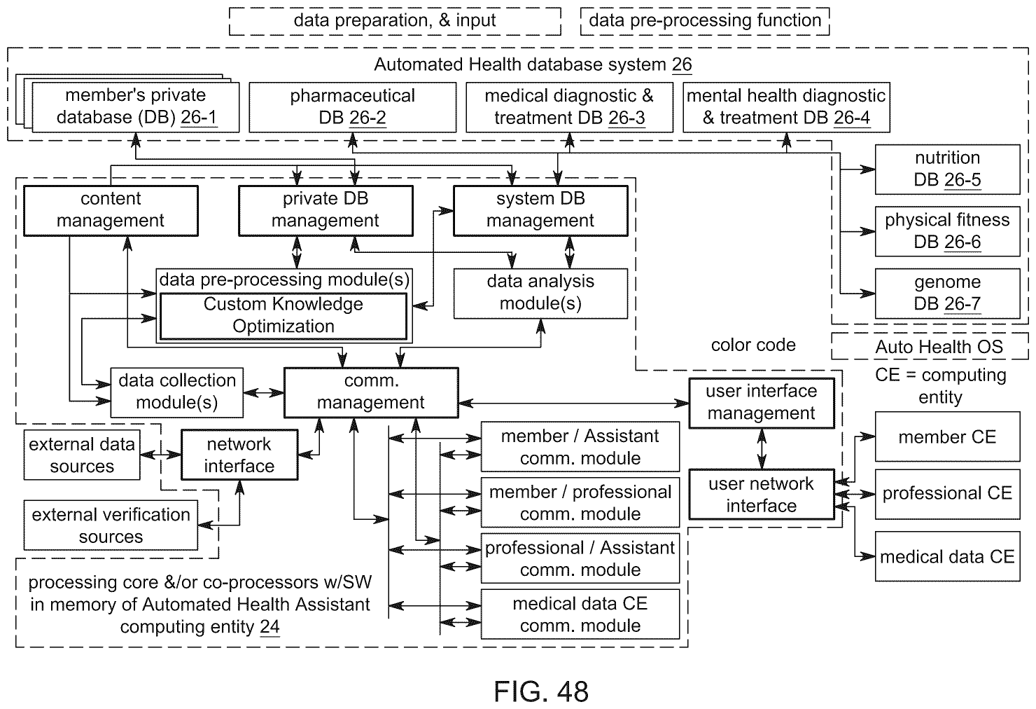

FIG. 48 is a schematic block diagram of an embodiment of computing system having an AI health assistant computing entity that processes customization of knowledge base data. In this example, the Custom Knowledgebase Building for Retrieval-Augmented Generation Optimization utilizes AI agents to rate the reliability and scientific rigor of various sources and integrates this data into a custom, any domain specific knowledgebase.

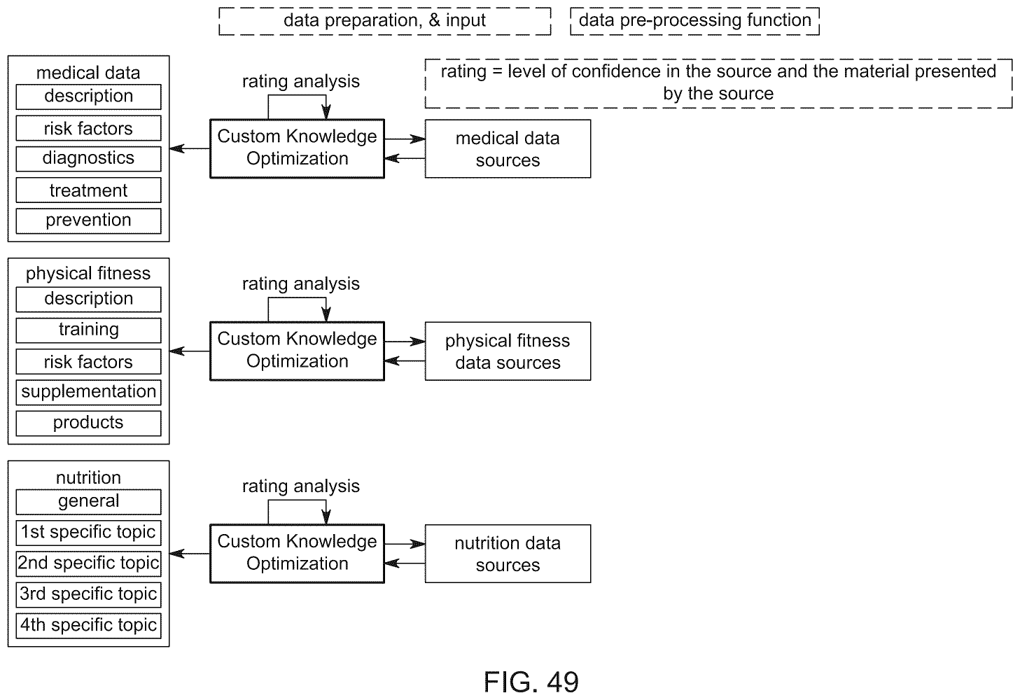

FIG. 49 is a schematic block diagram of another embodiment of computing system having an AI health assistant computing entity that processes customization of knowledge base data.

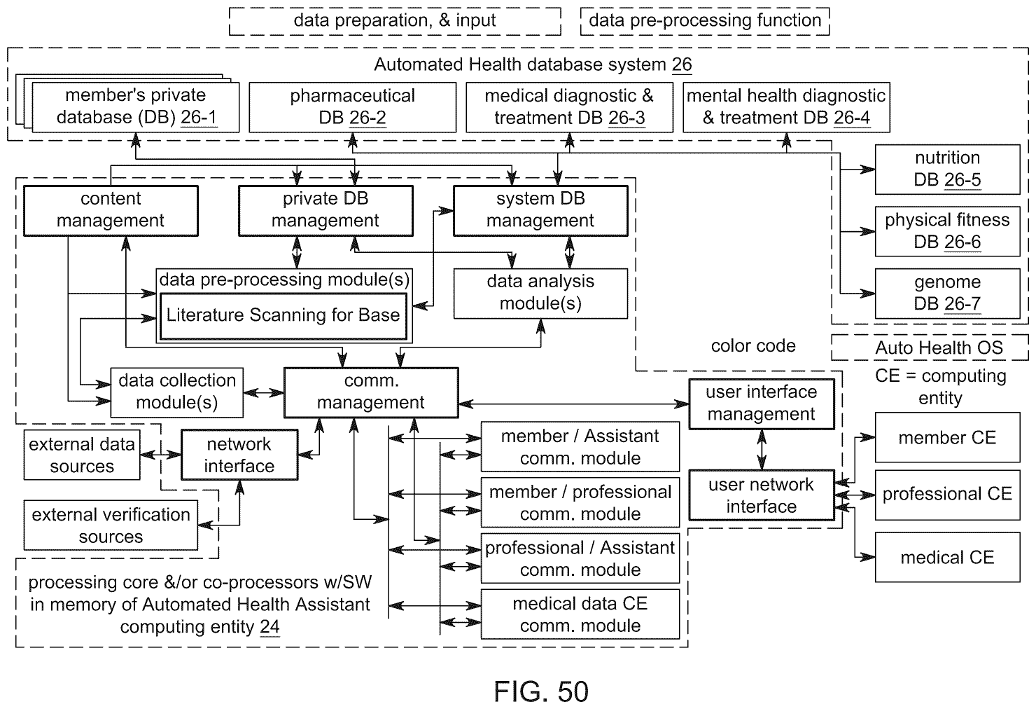

FIG. 50 is a schematic block diagram of an embodiment of computing system having an AI health assistant computing entity that processes scanning to expand knowledge base data. In this example, the Real-Time Literature Scanning Agents for Custom Knowledgebase scans and summarizes new publications, guidelines, or clinical trials for inclusion in a custom knowledgebase, extending its applicability beyond just the medical field.

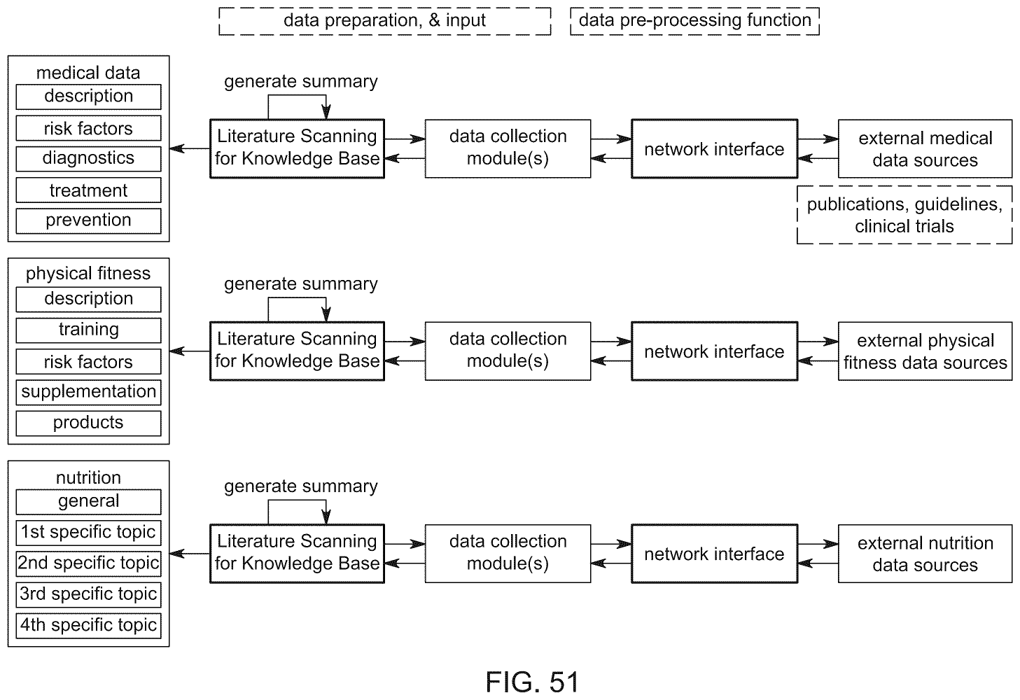

FIG. 51 is a schematic block diagram of another embodiment of computing system having an AI health assistant computing entity that processes scanning to expand knowledge base data.

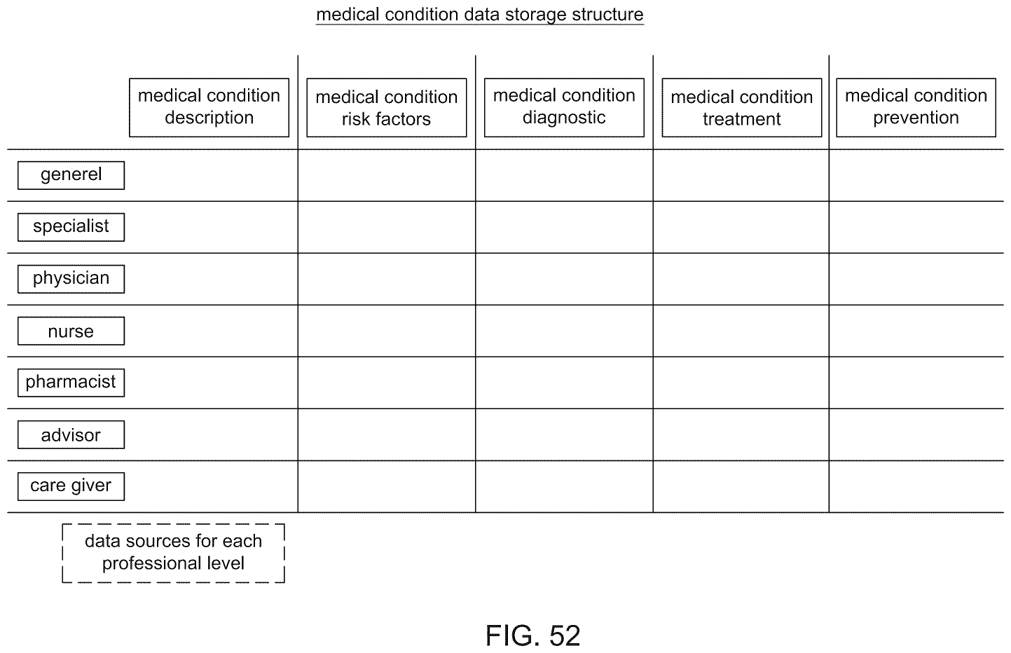

FIG. 52 is a schematic block diagram of an example of a medical data storage structure within a computing system having an AI health assistant computing entity. In an example, medical conditions are regarding primary medical matters, which include, but are not limited to: vascular disease, cancer, dementia, sarcopenia, and metabolic disease.

The medical data includes factors for the various medical conditions.

For example, several factors play a key role in the development of vascular disease. The primary ones are smoking, hypertension, high cholesterol-specifically Apolipoprotein B, and metabolic disease. Each of these elements contributes significantly to the problem.

Smoking has long been identified as a major health risk. It causes widespread damage throughout the body, reducing oxygen levels in the blood and increasing strain on the heart. It is a habit that has consistently been linked to poor health outcomes. Hypertension, or high blood pressure, is another key risk factor for vascular disease because it causes sustained damage to the blood vessels over time, making them more susceptible to blockages and other problems. Apolipoprotein B is a specific type of protein that carries cholesterol throughout the body. While cholesterol is essential for bodily functions, an excess of the type carried by Apo B can accumulate on the walls of blood vessels, forming plaques. These plaques can narrow the vessels and impede blood flow, increasing the risk of heart disease and stroke. Metabolic disease refers to conditions that disrupt the normal process of metabolizing food into energy. This can lead to a range of issues, including high blood sugar and obesity, both of which can increase the risk of vascular disease.

In another example, numerous factors contribute to the development of cancer, including lifestyle, genetic predisposition, and environmental exposures. However, a significant part of our fight against cancer revolves around early detection. Finding cancer at an early stage can often make a substantial difference in the disease's course, influencing treatment options, survival rates, and quality of life. Cancer screening refers to checking for cancer or precancerous conditions in people who do not have any symptoms. It is a proactive rather than reactive approach, a way of seeking out the enemy before it gains a stronghold. Regular screenings can help detect certain types of cancer at an earlier stage when they are more likely to be treatable and, often, curable. There are various screening methods available for diverse types of cancer. These include mammograms for breast cancer, colonoscopies for colorectal cancer, Pap tests and HPV tests for cervical cancer, and low dose computed tomography scans for lung cancer in high-risk individuals. Each of these screening methods has its own recommended schedule, often based on a person's age and risk factors.

In another example, dementia is not just an individual health issue, but a global one. It affects millions of people worldwide, causing a significant burden on patients, caregivers, and healthcare systems alike. Dementia is a term that encompasses several neurological disorders characterized by a decline in cognitive function beyond what might be expected from normal aging. This decline interferes with daily life and activities, affecting memory, thinking skills, behavior, and the ability to perform everyday tasks. While the risk of developing dementia increases with age, it is not an inevitable part of aging. Research has identified several risk factors for dementia, some of which can be modified. These include physical inactivity, smoking, unhealthy diet, excessive alcohol consumption, and medical conditions such as diabetes, high blood pressure, and high cholesterol.

In another example, sarcopenia is more than just a symptom of aging; it is a recognized medical condition that can lead to serious outcomes, such as physical disability, inadequate quality of life, and even death. While aging is the most significant risk factor for sarcopenia, other factors such as physical inactivity, inadequate protein intake, and chronic diseases can also contribute to its development.

In another example, metabolic diseases occur when the body's usual metabolic processes are disrupted. These conditions are often interconnected, with one often leading to another, creating a cascade of health problems. For example, obesity can lead to insulin resistance, which in turn can lead to type 2 diabetes and heart disease. Several risk factors contribute to metabolic diseases, including physical inactivity, unhealthy diet, smoking, excessive alcohol consumption, and genetic predisposition. However, many of these risk factors are modifiable, providing opportunities for prevention and intervention.

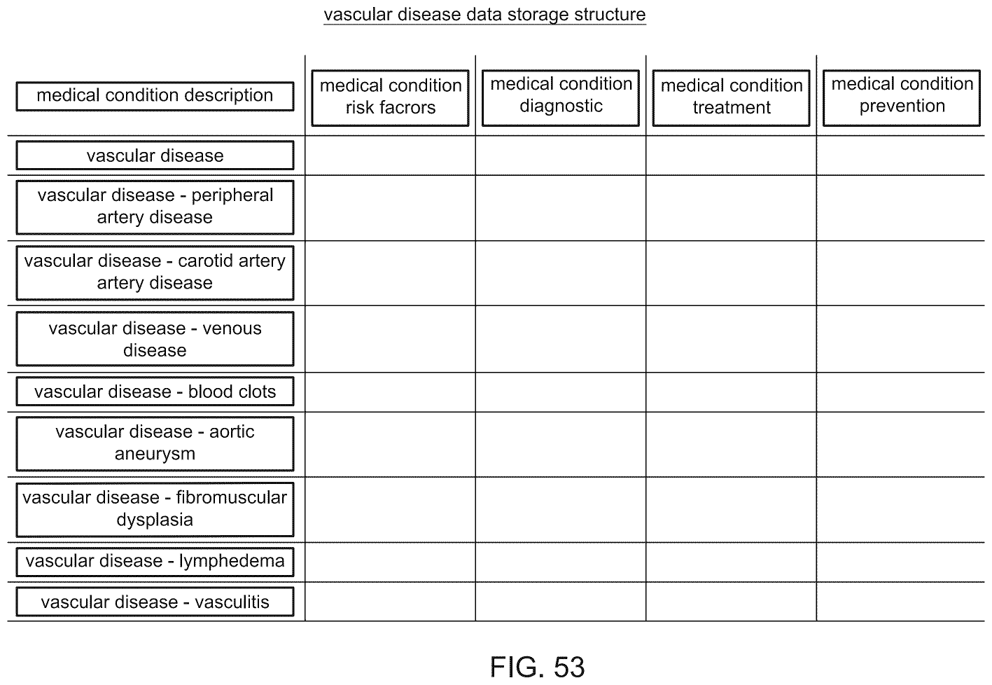

FIG. 53 is a schematic block diagram of an example of a vascular disease data storage structure within a computing system having an AI health assistant computing entity.

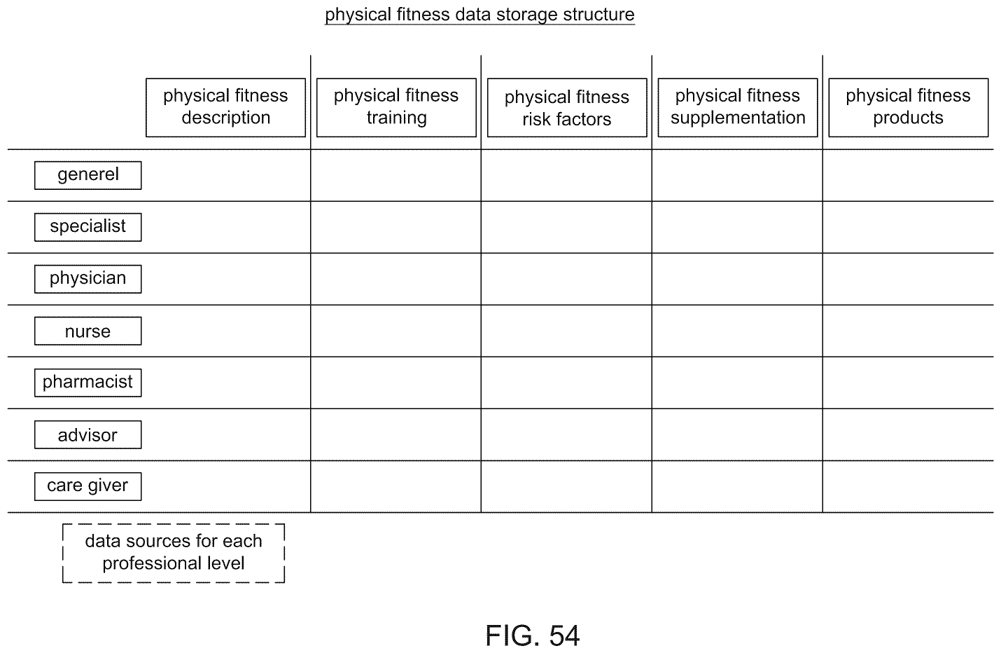

FIG. 54 is a schematic block diagram of an example of a physical fitness data storage structure within a computing system having an AI health assistant computing entity.

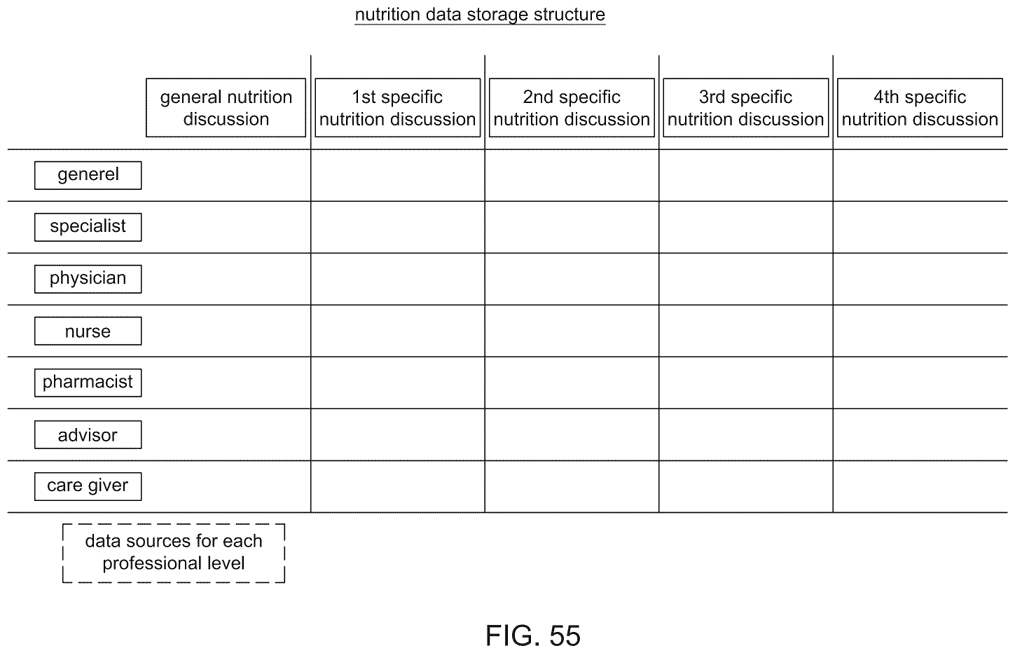

FIG. 55 is a schematic block diagram of an example of a nutrition data storage structure within a computing system having an AI health assistant computing entity.

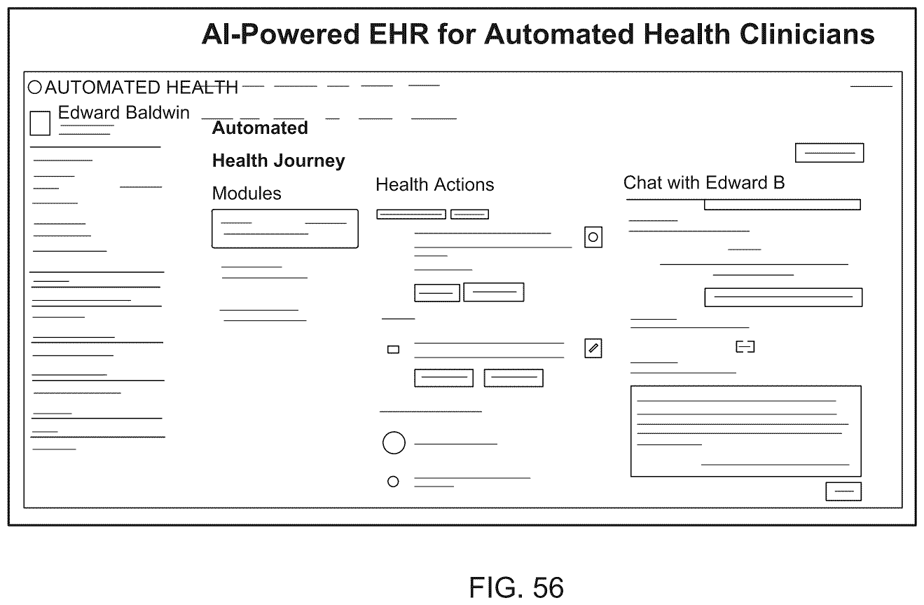

FIG. 56 is a schematic block diagram of an example of displaying a member's data within a computing system having an AI health assistant computing entity.

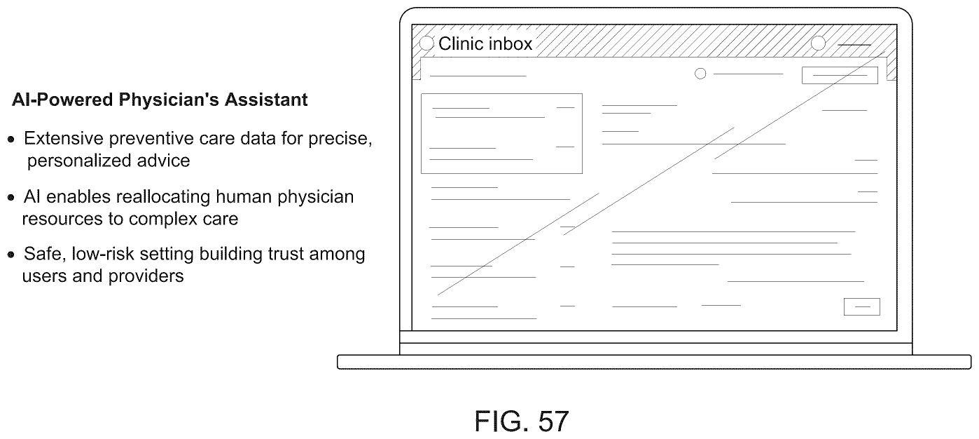

FIG. 57 is a schematic block diagram of another example of displaying a member's data within a computing system having an AI health assistant computing entity.

It is noted that terminologies as may be used herein such as bit stream, stream, signal sequence, etc. (or their equivalents) have been used interchangeably to describe digital information whose content corresponds to any of a number of desired types (e.g., data, video, speech, text, graphics, audio, etc. any of which may generally be referred to as ‘data’).

As may be used herein, the terms “substantially” and “approximately” provide an industry-accepted tolerance for its corresponding term and/or relativity between items. For some industries, an industry-accepted tolerance is less than one percent and, for other industries, the industry-accepted tolerance is 10 percent or more. Other examples of industry-accepted tolerance range from less than one percent to fifty percent. Industry-accepted tolerances correspond to, but are not limited to, component values, integrated circuit process variations, temperature variations, rise and fall times, thermal noise, dimensions, signaling errors, dropped packets, temperatures, pressures, material compositions, and/or performance metrics. Within an industry, tolerance variances of accepted tolerances may be more or less than a percentage level (e.g., dimension tolerance of less than +/−1%). Some relativity between items may range from a difference of less
than a percentage level to a few percent. Other relativity between items may range from a difference of a few percent to magnitude of differences.

As may also be used herein, the term(s) “configured to”, “operably coupled to”, “coupled to”, and/or “coupling” includes direct coupling between items and/or indirect coupling between items via an intervening item (e.g., an item includes, but is not limited to, a component, an element, a circuit, and/or a module) where, for an example of indirect coupling, the intervening item does not modify the information of a signal but may adjust its current level, voltage level, and/or power level. As may further be used herein, inferred coupling (i.e., where one element is coupled to another element by inference) includes direct and indirect coupling between two items in the same manner as “coupled to”.

As may even further be used herein, the term “configured to”, “operable to”, “coupled to”, or “operably coupled to” indicates that an item includes one or more of power connections, input(s), output(s), etc., to perform, when activated, one or more its corresponding functions and may further include inferred coupling to one or more other items. As may still further be used herein, the term “associated with”, includes direct and/or indirect coupling of separate items and/or one item being embedded within another item.

As may be used herein, the term “compares favorably”, indicates that a comparison between two or more items, signals, etc., provides a desired relationship. For example, when the desired relationship is that signal 1 has a greater magnitude than signal 2, a favorable comparison may be achieved when the magnitude of signal 1 is greater than that of signal 2 or when the magnitude of signal 2 is less than that of signal 1. As may be used herein, the term “compares unfavorably”, indicates that a comparison between two or more items, signals, etc., fails to provide the desired relationship.

As may be used herein, one or more claims may include, in a specific form of this generic form, the phrase “at least one of a, b, and c” or of this generic form “at least one of a, b, or c”, with more or less elements than “a”, “b”, and “c”. In either phrasing, the phrases are to be interpreted identically. In particular, “at least one of a, b, and c” is equivalent to “at least one of a, b, or c” and shall mean a, b, and/or c. As an example, it means: “a” only, “b” only, “c” only, “a” and “b”, “a” and “c”, “b” and “c”, and/or “a”, “b”, and “c”.

As may also be used herein, the terms “processing module”, “processing circuit”, “processor”, “processing circuitry”, and/or “processing unit” may be a single processing device or a plurality of processing devices. Such a processing device may be a microprocessor, micro-controller, digital signal processor, microcomputer, central processing unit, field programmable gate array, programmable logic device, state machine, logic circuitry, analog circuitry, digital circuitry, and/or any device that manipulates signals (analog and/or digital) based on hard coding of the circuitry and/or operational instructions. The processing module, module, processing circuit, processing circuitry, and/or processing unit may be, or further include, memory and/or an integrated memory element, which may be a single memory device, a plurality of memory devices, and/or embedded circuitry of another processing module, module, processing circuit, processing circuitry, and/or processing unit. Such a memory device may be a read-only memory, random access memory, volatile memory, non-volatile memory, static memory, dynamic memory, flash memory, cache memory, and/or any device that stores digital information. Note that if the processing module, module, processing circuit, processing circuitry, and/or processing unit includes more than one processing device, the processing devices may be centrally located (e.g., directly coupled together via a wired and/or wireless bus structure) or may be distributed (e.g., cloud computing via indirect coupling via a local area network and/or a wide area network). Further note that if the processing module, module, processing circuit, processing circuitry and/or processing unit implements one or more of its functions via a state machine, analog circuitry, digital circuitry, and/or logic circuitry, the memory and/or memory element storing the corresponding operational instructions may be embedded within, or external to, the circuitry comprising the state machine, analog circuitry, digital circuitry, and/or logic circuitry. Still further note that, the memory element may store, and the processing module, module, processing circuit, processing circuitry and/or processing unit executes, hard coded and/or operational instructions corresponding to at least some of the steps and/or functions illustrated in one or more of the Figures. Such a memory device or memory element can be included in an article of manufacture.

One or more embodiments have been described above with the aid of method steps illustrating the performance of specified functions and relationships thereof. The boundaries and sequence of these functional building blocks and method steps have been arbitrarily defined herein for convenience of description. Alternate boundaries and sequences can be defined so long as the specified functions and relationships are appropriately performed. Any such alternate boundaries or sequences are thus within the scope and spirit of the claims.

To the extent used, the flow diagram block boundaries and sequence could have been defined otherwise and still perform the certain significant functionality. Such alternate definitions of both functional building blocks and flow diagram blocks and sequences are thus within the scope and spirit of the claims. One of average skill in the art will also recognize that the functional building blocks, and other illustrative blocks, modules and components herein, can be implemented as illustrated or by discrete components, application specific integrated circuits, processors executing appropriate software and the like or any combination thereof.

In addition, a flow diagram may include a “start” and/or “continue” indication. The “start” and “continue” indications reflect that the steps presented can optionally be incorporated in or otherwise used in conjunction with one or more other routines. In addition, a flow diagram may include an “end” and/or “continue” indication. The “end” and/or “continue” indications reflect that the steps presented can end as described and shown or optionally be incorporated in or otherwise used in conjunction with one or more other routines. In this context, “start” indicates the beginning of the first step presented and may be preceded by other activities not specifically shown. Further, the “continue” indication reflects that the steps presented may be performed multiple times and/or may be succeeded by other activities not specifically shown. Further, while a flow diagram indicates a particular ordering of steps, other orderings are likewise possible provided that the principles of causality are maintained.

The one or more embodiments are used herein to illustrate one or more aspects, one or more features, one or more concepts, and/or one or more examples. A physical embodiment of an apparatus, an article of manufacture, a machine, and/or of a process may include one or more of the aspects, features, concepts, examples, etc. described with reference to one or more of the embodiments discussed herein. Further, from figure to figure, the embodiments may incorporate the same or similarly named functions, steps, modules, etc. that may use the same or different reference numbers and, as such, the functions, steps, modules, etc. may be the same or similar functions, steps, modules, etc. or different ones.

While transistors may be shown in one or more of the above-described figure(s) as field effect transistors (FETs), as one of ordinary skill in the art will appreciate, the transistors may be implemented using any type of transistor structure including, but not limited to, bipolar, metal oxide semiconductor field effect transistors (MOSFET), N-well transistors, P-well transistors, enhancement mode, depletion mode, and zero voltage threshold (VT) transistors.

Unless specifically stated to the contra, signals to, from, and/or between elements in a figure of any of the figures presented herein may be analog or digital, continuous time or discrete time, and single-ended or differential. For instance, if a signal path is shown as a single-ended path, it also represents a differential signal path. Similarly, if a signal path is shown as a differential path, it also represents a single-ended signal path. While one or more particular architectures are described herein, other architectures can likewise be implemented that use one or more data buses not expressly shown, direct connectivity between elements, and/or indirect coupling between other elements as recognized by one of average skill in the art.

The term “module” is used in the description of one or more of the embodiments. A module implements one or more functions via a device such as a processor or other processing device or other hardware that may include or operate in association with a memory that stores operational instructions. A module may operate independently and/or in conjunction with software and/or firmware. As also used herein, a module may contain one or more sub-modules, each of which may be one or more modules.

As may further be used herein, a computer readable memory includes one or more memory elements. A memory element may be a separate memory device, multiple memory devices, or a set of memory locations within a memory device. Such a memory device may be a read-only memory, random access memory, volatile memory, non-volatile memory, static memory, dynamic memory, flash memory, cache memory, and/or any device that stores digital information. The memory device may be in a form a solid-state memory, a hard drive memory, cloud memory, thumb drive, server memory, computing device memory, and/or other physical medium for storing digital information.

As applicable, one or more functions associated with the methods and/or processes described herein can be implemented via a processing module that operates via the non-human “artificial” intelligence (AI) of a machine. Examples of such AI include machines that operate via anomaly detection techniques, decision trees, association rules, expert systems and other knowledge-based systems, computer vision models, artificial neural networks, convolutional neural networks, support vector machines (SVMs), Bayesian networks, genetic algorithms, feature learning, sparse dictionary learning, preference learning, deep learning and other machine learning techniques that are trained using training data via unsupervised, semi-supervised, supervised and/or reinforcement learning, and/or other AI. The human mind is not equipped to perform such
AI techniques, not only due to the complexity of these techniques, but also due to the fact that artificial intelligence, by its very definition-requires “artificial” intelligence—i.e., machine/non-human intelligence.

As applicable, one or more functions associated with the methods and/or processes described herein can be implemented as a large-scale system that is operable to receive, transmit and/or process data on a large-scale. As used herein, a large-scale refers to a large number of data, such as one or more kilobytes, megabytes, gigabytes, terabytes or more of data that are received, transmitted and/or processed. Such receiving, transmitting and/or processing of data cannot practically be performed by the human mind on a large-scale within a reasonable period of time, such as within a second, a millisecond, microsecond, a real-time basis or other high speed required by the machines that generate the data, receive the data, convey the data, store the data and/or use the data.

As applicable, one or more functions associated with the methods and/or processes described herein can require data to be manipulated in different ways within overlapping time spans. The human mind is not equipped to perform such different data manipulations independently, contemporaneously, in parallel, and/or on a coordinated basis within a reasonable period of time, such as within a second, a millisecond, microsecond, a real-time basis or other high speed required by the machines that generate the data, receive the data, convey the data, store the data and/or use the data.

As applicable, one or more functions associated with the methods and/or processes described herein can be implemented in a system that is operable to electronically receive digital data via a wired or wireless communication network and/or to electronically transmit digital data via a wired or wireless communication network. Such receiving and transmitting cannot practically be performed by the human mind because the human mind is not equipped to electronically transmit or receive digital data, let alone to transmit and receive digital data via a wired or wireless communication network.

As applicable, one or more functions associated with the methods and/or processes described herein can be implemented in a system that is operable to electronically store digital data in a memory device. Such storage cannot practically be performed by the human mind because the human mind is not equipped to electronically store digital data.

While particular combinations of various functions and features of the one or more embodiments have been expressly described herein, other combinations of these features and functions are likewise possible. The present disclosure is not limited by the particular examples disclosed herein and expressly incorporates these other combinations.

1. A computing system for medical data processing, comprising:
a plurality of member computing entities each storing and executing a member application configured to track, execute, and communicate a member's dynamic wellness plan;
a health assistant computing entity coupled to the plurality of member computing entities via a network, wherein the automated health assistant computing entity includes:
a processor configured to execute a machine learning and artificial intelligence application that uses model inference to apply a trained model for facilitating member wellness functions;
a private database for storing member-specific data;
an automated health operating system, the operating system including modules for private database management, automated health content management, communication management, and security management; and
an automated health database system coupled to the automated health assistant computing entity via a private network, the database system comprising a plurality of databases storing health-related data including medical diagnosis, pharmaceutical data, mental health data, nutrition data, physical fitness data, and genetic data,
wherein the health assistant computing entity is configured to:
process and analyze the member-specific data using the machine learning and artificial intelligence programs;
generate personalized health insights and treatment plans based on the analyzed data;
facilitate communication between the member computing entities and professional computing entities to coordinate health and wellness actions; and
monitor and update the member's wellness plan in real-time based on new data inputs.

2. The computing system of claim 1, wherein the private network connecting the automated health assistant computing entity to the automated health database system is a virtual private network (VPN).

3. The computing system of claim 1, further comprising a plurality of professional computing entities each storing and executing an automated professional application configured to interact with the automated health assistant computing entity and member computing entities.

4. The computing system of claim 1, wherein the machine learning and artificial intelligence programs include modules for predictive analysis, treatment recommendation, and health risk assessment.

5. The computing system of claim 1, wherein the automated health operating system further includes a module for interoperability of data, enabling the integration of external health data sources into the automated health database system.

6. The computing system of claim 1, wherein the member computing entities are selected from the group consisting of smartphones, tablets, laptops, and wearable devices.

7. The computing system of claim 1, wherein the automated health assistant computing entity is further configured to perform behavioral health monitoring through voice and text analysis.

8. The computing system of claim 1, wherein the automated health database system includes a genome database for storing and processing genetic data related to the member.

9. The computing system of claim 1, wherein the automated health assistant computing entity is further configured to interface with external health information exchanges (HIEs) for retrieving additional medical data.

10. The computing system of claim 1, further comprising a data communication module configured to encrypt all communications between the member computing entities, professional computing entities, and the automated health assistant computing entity.

11. A method for personalized medical data processing, comprising:
receiving, by a health assistant computing entity, member-specific health data from a plurality of member computing entities via a network;
processing the received health data using machine learning and artificial intelligence algorithms executed by the health assistant computing entity to generate personalized health insights;
storing the processed health data and generated insights in a private database associated with the automated health assistant computing entity;
generating a dynamic wellness plan for each member based on the processed data and insights;
facilitating communication between the member computing entities and professional computing entities to implement the wellness plan;
continuously monitoring the member's health data and dynamically updating the wellness plan based on new data inputs; and
providing real-time feedback and recommendations to the member through the member computing entities.

12. The method of claim 11, further comprising the step of validating the generated health insights and recommendations through a professional computing entity before presenting them to the member.

13. The method of claim 11, wherein the health data received from the member computing entities includes data from wearable health monitoring devices.

14. The method of claim 11, further comprising the step of performing health risk assessment by comparing the member-specific health data against population-level health data stored in the automated health database system.

15. The method of claim 11, wherein the real-time feedback provided to the member includes alerts for potential health risks detected by the machine learning algorithms.

16. The method of claim 11, wherein the dynamic wellness plan includes personalized recommendations for physical fitness, nutrition, mental health, and medical treatments.

17. The method of claim 11, further comprising aggregating health data from multiple members to improve the accuracy of the machine learning model used by the automated health assistant computing entity.

18. The method of claim 11, wherein continuously monitoring the member's health data includes detecting changes in the member's biometrics and adjusting the wellness plan accordingly.

19. The method of claim 11, wherein the automated health assistant computing entity generates wellness plan compliance reports for the member based on tracked adherence to the recommendations.

20. The method of claim 11, wherein the machine learning algorithms used for processing the health data are trained on anonymized historical health data stored in the automated health database system.
